(19) United States
(12) Patent Application Publication
(10) Pub. No.: US 2008/0226750 A1
(43) Pub. Date: Sep. 18, 2008

(54) Methods, Compositions and Articles of Manufacture for Treating Shock and Other Adverse Conditions

(75) Inventors: Mark B. Roth, Seattle, WA (US); Mike Morrison, Seattle, WA (US)

Correspondence Address: FULBRIGHT & JAWORSKI L.L.P., 600 CONGRESS AVE., SUITE 2400, AUSTIN, TX, 78701, US

(73) Assignee: FRED HUTCHINSON CANCER RESEARCH CENTER

(21) Appl. No.: 11/738,294

(22) Filed: Apr. 20, 2007

Related U.S. Application Data
(60) Provisional application No. 60/793,520, filed on Apr. 20, 2006.
(60) Provisional application No. 60/869,054, filed on Dec. 7, 2006.

(51) Int. Cl. A61K 33/04; A61P 7/00

(52) U.S. Cl. 424/708

(57) ABSTRACT

The present invention concerns the use of active compounds for inducing apnea and treating shock, in addition to enhancing the survivability of a subject. It includes compositions, methods, articles of manufacture and apparatuses for enhancing survivability and for achieving these effects.

This application claims the benefit of priority to U.S. Provisional Application Serial No. 66/793,520 filed on Apr. 20, 2006 and U.S. Provisional Application Ser. No. 60/869,054 filed on Dec. 7, 2006, both of which are hereby incorporated by reference.

The government may own rights in the present invention pursuant to grant number GM048435 from the National Institute of General Medical Sciences (NIGMS).

### BACKGROUND OF THE INVENTION

1. Field of the Invention

The present invention relates generally to the field of trauma care and physiology. More particularly, it concerns methods, compositions and apparatuses for enhancing the survivability of organisms, particularly under adverse conditions, such as those leading to shock. In certain embodiments, there are methods, compositions and apparatuses for treating a subject at risk for hemorrhagic shock using an agent (referred to as an “active compound”). In specific embodiments, the agent is a gas, such as hydrogen sulfide (H₂S), or in a liquid or other formulation.

2. Description of Related Art

Shock is a life-threatening condition that progresses rapidly when interventions are delayed. Shock is a state in which adequate perfusion to sustain the physiologic needs of organ tissues is not present. This is a condition of profound haemodynamic and metabolic disturbance characterized by failure of the circulatory system to maintain adequate perfusion of vital organs. It may result from inadequate blood volume (hypovolaemic shock), inadequate cardiac function (cardiogenic shock) or inadequate vasomotor tone, also referred to as distributive shock (neurogenic shock, septic shock, anaphylactic shock). This often results in rapid mortality of the patient. Many conditions, including sepsis, blood loss, impaired autoregulation, and loss of autonomic tone, may produce shock or shocklike states. The present invention is anticipated to prevent detrimental effects of all the above states of shock, and sustain the life of the biological matter undergoing such shock.

According to an article by Dr. William Bozeman (eMedicine.com), hemorrhagic shock occurs when so much blood is lost that the body is unable to compensate and provide adequate tissue perfusion and oxygenation. Trauma is often the cause, but it may also be caused by spontaneous hemorrhage, such as from gastrointestinal bleeding, childbirth, or surgery. An acute bleeding episode with a discrete precipitating event is most often the cause of clinical hemorrhagic shock, though it is occasionally caused by chronic conditions with subacute blood loss.

The body's physiological response to hemorrhage includes mechanisms such as initial peripheral and mesenteric vasoconstriction to shunt blood to the central circulation. Progressive tachycardia also increases these effects. An increase in cardiac index, oxygen delivery (i.e., DO₂), increased oxygen consumption (i.e., VO₂) by tissues may be observed in a patient in hemorrhagic shock. An evaluation of lactate levels, the acid-base status, and other markers may also contribute helpful information regarding physiological status. A subject's response to hemorrhagic shock can vary significantly depending on their age, with those very young or very old being most prone to early decompensation after blood loss.

Death can result when the compensatory mechanisms are unable to compensate for the blood loss. In the absense of medical intervention, a classic trimodal distribution of deaths is seen with patients experiencing severe hemorrhagic shock. This distribution shows an initial peak of mortality due to immediate blood loss that occurs within minutes of hemorrhage. A second peak is observed in the time period of one to several hours due to progressive decompensation. Finally, there is a third peak that occurs days to weeks later as a result of sepsis and organ failure.

According the 2004 National Vital Statistics Report, in 2001 more than 150,000 people in the United States died from injuries they sustained. Of these, 64.6 percent were classified as unintentional, 19.5 percent were suicides, 12.9 percent were homicides, 2.7 percent were of undetermined intent, and 0.3 percent involved legal intervention or operations of war. Motor vehicle traffic, firearm, and falls were the three leading causes of these deaths. A large proportion of these fatalities result from massive blood loss due to the trauma, leading to hemorrhagic shock.

While the majority of trauma injury cases do not require surgery or care by a trauma service, some patients with serious injuries need stabilization within the “Golden Hour” post-injury, to improve their chances of survival and to minimize disability.

As most shock cases are due to injury caused by an accident, pre-hospital care is critical to the survival of the patient. This may include rapid assessment, stabilization, and expeditious transport to an appropriate facility for evaluation and treatment.

In all patients with shock syndrome, the maintenance of a patent airway, adequate breathing and adequate circulation are the primary focus of emergency treatment. Assessment is essential, as changes in client condition indicate progression of the shock syndrome. Early intervention is vital to minimize damage to tissues and organs and minimize permanent disability and early identification of the primary clinical cause is critical. Treatments are directed toward correcting the cause of the shock syndrome and slowing progression. Intravenous access and fluid resuscitation (typically IV saline) are standard, however, there is some debate over this because quick reversal of hypervolemia may cause hemorrhage increase, clotting factors to become diluted, or partially formed clots to become dislodged. When hypovolemic shock results from massive hemorrhage, the replacement fluid of choice is whole blood or packed red blood cells. Crystalloid solutions will temporarily improve circulating volume, but the patient also needs replacement of red blood cells to carry oxygen to the tissues. Management of shock focuses on fluid management, acid-base balance, and improving myocardial contraction. Treating the underlying cause of shock should also be treated in order to diminish the progression of the shock syndrome.

There are two major stages of shock: early compensation stage and progressive stage. It is contemplated that embodiments of the invention may be applied to patients in either or both stages.

Whole body hibernation was induced in mice, and there was an immediate drop in overall metabolic state (as measured by CO₂ evolution). This was reversible, and the mice seem to function normally, even after repeated exposures. Accordingly, the invention concerns inducing a whole body hibernetic state using H₂S (or other oxygen antagonist or other active compound), to preserve the patient's vital organs and life. This will allow for transport to a controlled environment (e.g., surgery), where the initial cause of the shock can be addressed, and then the patient brought back to normal function in a controlled manner. For this indication, the first hour after injury, referred to as the “golden hour,” is crucial to a successful outcome. Stabilizing the patient in this time period is the major goal, and transport to a critical care facility (e.g., emergency room, surgery, etc.) where the injury can be properly addressed. Thus, it would be ideal to maintain the patient in a state that would allow for this and to address immediate concerns such as source of shock, replenish blood loss, and reestablish homeostasis. There is a need for such treatment to increase the likelihood of surviving hemorrhagic shock.

### SUMMARY OF THE INVENTION

Therefore, the present invention provides methods, compositions, articles of manufacture, and apparatuses to treat, prevent, or enhance the chances of surviving shock and other adverse conditions. Such methods, compositions, articles of manufacture, and apparatuses can be employed to protect a subject, including where the subject is a human, from succumbing to shock or other adverse conditions. In addition, such aspects of the invention may reduce the risks and negative outcomes from, for example, shock, including death.

In certain embodiments of the invention there are methods for inducing apnea in a subject comprising providing to the subject an effective amount of an active compound. The term “apnea” is used according to its ordinary meaning to refer to a “period of time during which breathing is markedly reduced such that the subject takes 10% or fewer number of breaths.”

Other aspects of the invention concern methods for preventing an organism from bleeding to death comprising providing to the bleeding organism an effective amount of an active compound to prevent death.

In other methods of the invention, there are methods for protecting biological subject from an injury (which may or may not be iatrogenic or non-iatrogenic), the onset or progression of a disease, or death comprising providing to the subject, before the injury, the onset or progression of a disease, or death, an effective amount of an active compound, wherein the effective amount is less than an amount that can induce stasis in the biological subject.

Also contemplated as part of the invention is a method for inducing apnea in a subject comprising administering to a subject an effective amount of gaseous chalcogenide, salt or prodrug thereof to the subject.

Additionally, other methods of the invention include methods for treating hemorrhagic shock in a patient comprising providing an effective amount of gaseous hydrogen sulfide. The term “patient” is used to refer to a human subject.

Even further methods of the invention relate to methods for preventing or treating shock in a subject comprising providing to a subject an effective amount of gaseous chalcogenide, salt or prodrug thereof to the subject.

The different embodiments below pertain to, but are not limited to, these methods of the invention.

An active compound refers to a compound that can achieve the stated goal. In certain embodiments, the active compound is an oxygen antagonist. In other embodiments, it is or is also a protective metabolic agent. In further embodiments, it may not be an oxygen antagonist and/or it may not be a protective metabolic agent. Any embodiment discussed with respect to an oxygen antagonist or a protective metabolic agent may be generally implemented with respect to any active compound, and vice versa.

In particular embodiments, the active compound is a chalcogenide or chalcogenide salt. Moreover, the active compound may have a chemical structure of Formula I, Formula II, Formula III, or Formula IV, which are discussed below. In certain cases, the subject is provided with a precursor of a chemical structure of Formula I, Formula II, Formula III, or Formula IV. In even further embodiments, the active compound has a chemical formula of Formula II(a), Formula II(b), or Formula II(c), while in others, the active compound has a chemical formula of Formula III(a), Formula III(b), Formula III(c), Formula III(d), Formula III(e), Formula III(f), Formula III(g), or Formula III(h).

In particular embodiments, the active compound comprises sulfur and/or selenium. In further embodiments, the active compound comprises a chalcogenide or chalcogenide salt. In certain cases, the chalcogenide or chalcogenide salt is selected from the group consisting of H₂S, Na₂S, NaHS, K₂S, KHS, Rb₂S, Cs₂S, (NH₄)₂S, (NH₄)HS, BeS, MgS, CaS, SrS, and BaS.

In some methods of the invention, the subject is provided with a combination of active compounds.

The present invention concerns embodiments in which the subject is provided with the active compound before, during, or after an injury, the onset or progression of trauma, or hemorrhaging in the subject. In specific embodiments, the injury involves hemorrhaging. Moreover, the injury may be from an external physical source.

In particular instances, the active compound is provided before the injury or before the onset or progression of the disease. Moreover, in other embodiments, the active compound is not provided during or after the injury or the onset or progression of the disease.

In certain situations, the subject is bleeding or is at risk for bleeding. Moreover, the subject may be in shock or at risk for being in shock.

It is contemplated that in some embodiments, the subject is exposed to one of the oxygen antagonists for a period of time between about 10 seconds and about 1 hour.

In some embodiments, the subject becomes apneic after being provided the chalcogenide, salt, or prodrug thereof. In additional embodiments, the subject ceases skeletal muscle movement after being provided the chalcogenide, salt, or prodrug thereof.

Some methods involve providing a subject or patient with a single dose of the chalcogenide, salt, or prodrug thereof. In other methods of the invention, a patient is provided with the chalcogenide, salt, or prodrug thereof by inhaling a composition comprising the chalcogenide, salt, or prodrug thereof. Moreover, in further embodiments, methods involve subsequently providing to the patient a gaseous composition that does not include the chalcogenide, salt, or prodrug thereof. This may composition may include, for instance, oxygen.

The invention is based, in part, on studies with compounds that were determined to have a protective function, and thus, serve as protective agents. Moreover, the overall results of studies involving different compounds indicate that certain compounds can induce certain physiological states. In certain embodiments, these compounds are particularly effective in inducing apnea, stasis or pre-stasis. In addition, these compounds induce reversible stasis, meaning they are not so toxic to the particular biologic matter that the matter dies or decomposes. It is further contemplated that the present invention can be used to enhance survivability of and/or to prevent or reduce damage to biological matter, which may be subject to or under adverse conditions. This subject matter is described in U.S. Provisional Patent Application 60/827,337, filed Sep. 28, 2006; U.S. Provisional Patent Applications 60/673,037 and 60/673,295 both filed on Apr. 20, 2005, as well as U.S. Provisional Patent Application 60/713,073, filed Aug. 31, 2005, U.S. Provisional Patent Application 60/731,549, filed Oct. 28, 2005, and U.S. Provisional Patent Application 60/762,462, filed on Jan. 26, 2006 and a nonprovisional U.S. patent application claiming the benefit of these applications and filed on Apr. 20, 2006, in the names of Mark Roth, Mike Morrison, and Eric Blackstone, all of which are hereby incorporated by reference herein for their teaching about active compounds, dosages, modes of administration, applications, and proof of concept experiments.

Some methods of the invention involve what is generally referred to as “pre-treatment” with an active compound. Pre-treatment includes methods wherein biological matter is provided with an active compound both before and during, and before, during and after biological matter is subjected to adverse conditions (e.g., an injury or onset or the progression of a disease), and methods wherein biological matter is provided with an active compound only before biological matter is subjected to adverse conditions. It is generally contemplated that pre-treatment can involve providing an active compound, before, during, and/or after biological matter is subjected to adverse conditions. In some embodiments, the active compound is provided before and after being subjected to adverse conditions.

In particular embodiments, treatment with an active compound induces “pre-stasis,” which refers to a hypometabolic state through which biological matter must transition to reach stasis. Pre-stasis is characterized by a reduction in metabolism within the biological material of a magnitude that is less than that defined as stasis. In order to achieve stasis using an active compound, the biological matter necessarily must transition through a graded hypometabolic state in which oxygen consumption and CO₂ production are reduced less than two-fold in the biological matter. Such a continuum, in which metabolism or cellular respiration is reduced by an active compound to a degree less than two-fold, can be described as a state of “pre-stasis.”

To the extent that stasis comprises a two-fold reduction (i.e., a reduction to 50% or less) in either CO₂ production or O₂ consumption, direct measurement of these parameters in the biological matter using methods known to those in the art in which a reduction of less than two-fold is detected is indicative of pre-stasis. Accordingly, certain measurements of carbon dioxide and oxygen levels in the blood as well as other markers of metabolic rate familiar to those skilled in the art including, but not limited to, blood pO₂, VO₂, pCO₂, pH, and lactate levels, may be used in the instant invention to monitor the onset or progression of pre-stasis. While indicators of metabolic activity, e.g., CO₂ production via cellular respiration and O₂ consumption, are reduced less than two-fold as compared to normal conditions, pre-stasis may be associated with an at least 10%, 15%, 20%, 25%, 30%, 35%, 40%, 45%, or 50% reduction in CO₂ evolution, which refers to the amount of CO₂ released from the lungs. In addition, in various embodiments, pre-stasis is characterized by a reduction in one or more indicators of metabolic activity that is less than or equal to 1%, 2%, 5%, 10%, 15%, 20%, 25%, 30%, 35%, 40%, 45%, or 50% as compared to normal physiological conditions. In other embodiments, pre-stasis is characterized by its ability to enhance or promote entry into stasis in response to another stimuli (wherein the another stimuli may include prolonged treatment with the same active agent), or its ability to enhance survival of or protect biological matter from damage resulting from an injury, the onset or progression of the disease, or bleeding, particularly bleeding that can lead to irreversible tissue damage, hemorrhagic shock, or lethality.

While methods of the present invention explicitly exemplified herein may refer to inducing “stasis,” it is understood that these methods may be readily adapted to induce “pre-stasis,” and that such methods of inducing pre-stasis are contemplated by the present invention. In addition, the same active compounds used to induce stasis may also be used to induce pre-stasis, by providing them to biological matter at a lower dosage and/or for a shorter time than used to induce stasis.

In certain embodiments, the present invention involves exposing biological matter to an amount of an agent, so as to achieve stasis of the biological matter. In some embodiments, the present invention concerns methods for inducing stasis in in vivo biological matter comprising: a) identifying an organism in which stasis is desired; and, b) exposing the organism to an effective amount of an active compound to induce stasis in the in vivo biological matter. Inducing “stasis” in biological matter means that the matter is alive but is characterized by one or more of the following: at least a two-fold reduction in the rate or amount of carbon dioxide production by the biological matter; at least a two-fold (i.e., 50%) reduction in the rate or amount of oxygen consumption by the biological matter; and at least a 10% decrease in movement or motility (applies only to cells or tissue that move, such as sperm cells or a heart or a limb, or when stasis is induced in the entire organism) (collectively referred to as “cellular respiration indicators”). In certain embodiments of the invention, it is contemplated that there is about, at least about, or at most about a 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, 1000-, 1100-, 1200-, 1300-, 1400-, 1500-, 1600-, 1700-, 1800-, 1900-, 2000-, 2100-, 2200-, 2300-, 2400-, 2500-, 2600-, 2700-, 2800-, 2900-, 3000-3.100-, 3200-, 3300, 3400-, 3500-, 3600-, 3700-, 3800-, 3900-, 4000-, 4100-, 4200-, 4300-, 4400-, 4500-, 5000-, 6000-, 7000-, 8000-, 9000-, or 10000-fold or more reduction in the rate of oxygen consumption by the biological matter, or any range derivable therein. Alternatively, it is contemplated that embodiments of the invention may be discussed in terms of a reduction in the rate of oxygen consumption by the biological matter as about, at least about, or at most about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein. It is contemplated that any assay to measure oxygen consumption may be employed, and a typical assay will involve utilizing a closed environment and measuring the difference between the oxygen put into the environment and oxygen that is left in the environment after a period of time. It is further contemplated that carbon dioxide production can be measured to determine the amount of oxygen consumption by biological matter. Thus, there may be decreases in carbon dioxide production, which would correspond to the decreases in oxygen consumption discussed above.

In methods of the invention, stasis or pre-stasis is temporary and/or reversible, meaning that the biological matter no longer exhibits the characteristics of stasis at some later point in time. In some embodiments of the invention, instead of an oxygen antagonist, a compound that is not does not qualify as an oxygen antagonist is administered. It is contemplated that methods discussed with respect to oxygen antagonists may be applied with respect to any compound that is an oxygen antagonist, protective metabolic agent, compound with the structure of Formula I, II, III, or IV, any other active compound discussed herein, or a salt or precursor thereof. A compound that achieves any method of the invention may qualify as an oxygen antagonist, protective metabolic agent, compound with the structure of Formula I, II, III, or IV, or a salt or precursor thereof, all of which may be considered an “active compound.” In particular embodiments, induction of stasis is desired in which case the compound may be referred to as an “active stasis compound.” It is contemplated that in some embodiments of the invention, a method is achieved by inducing stasis. For example, therapeutic methods may involve inducing stasis, in which case the active compound is an active stasis compound. It is specifically contemplated that in embodiments in which active compounds are discussed, the invention includes, and may be limited to, oxygen antagonists.

In certain embodiments of the present invention, biological matter is treated with an active compound that does not induce stasis by itself (at least not at the level and/or duration of time provided), but rather induces biological matter to enter a pre-stasis state that has therapeutic benefits and that enhances the ability of the biological matter to achieve stasis in response to another stimuli, such as, e.g., an injury, disease state, or treatment with another active compound or the same active compound If used for a longer duration or greater dosage.

The term “biological matter” refers to any living biological material (mammalian biological material in preferred embodiments) including cells, tissues, organs, and/or organisms, and any combination thereof. It is contemplated that stasis may be induced in a part of an organism (such as in cells, in tissue, and/or in one or more organs), whether that part remains within the organism or is removed from the organism, or the whole organism will be placed in a state of stasis. Moreover, it is contemplated in the context of cells and tissues that homogenous and heterogeneous cell populations may be the subject of embodiments of the invention. The term “in vivo biological matter” refers to biological matter that is in vivo, i.e., still within or attached to an organism. Moreover, the term “biological matter” will be understood as synonymous with the term “biological material.” In certain embodiments, it is contemplated that one or more cells, tissues, or organs is separate from an organism. The term “isolated” can be used to describe such biological matter. It is contemplated that stasis may be induced in isolated biological matter.

An organism or other biological matter in need of stasis is an organism or biological matter in which stasis of all or part of the organism may yield direct or indirect physiological benefits. For example, a patient at risk for hemorrhagic shock may be considered in need of stasis, or a patient who will undergo coronary artery bypass surgery may benefit from protecting the heart from ischemia/reperfusion injury. Other applications are discussed throughout the application. In some cases, an organism or other biological matter is identified or determined to be in need of stasis based on one or more tests, screens, or evaluations that indicate a condition or disease, or the risk of a condition or disease that can be prevented or treated by undergoing stasis. Alternatively, the taking of a patient medical or family medical history (patient interview) may yield information that an organism or other biological matter is in need of stasis. As would be evident to one skilled in the art, one application of the present invention would be to reduce the overall energy demands of a biological material by inducing stasis.

Alternatively, an organism or other biological matter may be in need of an active compound to enhance survivability. For instance, a patient may need treatment for an injury or disease or any other application discussed herein. They may be determined to be in need as disclosed in the previous paragraph with respect to the need to enhance survivability or in need of treatment.

The term “oxygen antagonist” refers to a substance that competes with oxygen insofar as it is used by a biological matter that requires oxygen for it to be alive (“oxygen-utilizing biological matter”). Oxygen is typically used or needed for various cellular processes that create the biological matter's primary source of readily utilizable energy. An oxygen antagonist effectively reduces or eliminates the amount of oxygen that is available to the oxygen-utilizing biological matter, and/or the amount of oxygen that can be used by the oxygen-utilizing biological matter. In one embodiment, an oxygen antagonist may achieve its oxygen antagonism directly. In another embodiment, an oxygen antagonist may achieve its oxygen antagonism indirectly.

A direct oxygen antagonist competes with molecular oxygen for the binding to a molecule (e.g., a protein) that has an oxygen binding site or oxygen binding capacity. Antagonism may be competitive, non-competitive, or uncompetitive as known in the art of pharmacology or biochemistry. Examples of active compounds that are direct oxygen antagonists include, but are not limited to, carbon monoxide (CO), which competes for oxygen binding to hemoglobin and to cytochrome c oxidase.

An indirect oxygen antagonist influences the availability or delivery of oxygen to cells that use oxygen for energy production (e.g., in cellular respiration) in the absence of directly competing for the binding of oxygen to an oxygen-binding molecule. Examples of indirect oxygen antagonists include, but are not limited to, (i) carbon dioxide, which, through a process known as the Bohr effect, reduces the capacity of hemoglobin (or other globins, like myoglobin) to bind to oxygen in the blood or hemolymph of oxygen-utilizing animals, thereby reducing the amount of oxygen that is delivered to oxygen-utilizing cells, tissues, and organs of the organism, thereby reducing the availability of oxygen to cells that use oxygen; (ii) inhibitors of carbonic anhydrase (Supuran et al., 2003, incorporated by reference in its entirety) which, by virtue of inhibiting the hydration of carbon dioxide in the lungs or other respiratory organs, increase the concentration of carbon dioxide, thereby reducing the capacity of hemoglobin (or other globins, like myoglobin) to bind to oxygen in the blood or hemolymph of oxygen-utilizing animals, thereby reducing the amount of oxygen that is delivered to oxygen-utilizing cells, tissues, and organs of the organism, thereby reducing the availability of oxygen to cells that use oxygen; and, (iii) molecules that bind to oxygen and sequester it from or rendering it unavailable to bind to oxygen-binding molecules, including, but not limited to oxygen chelators, antibodies, and the like.

In some embodiments, an oxygen antagonist is both a direct and an indirect oxygen antagonist. Examples include, but are not limited to, compounds, drugs, or agents that directly compete for oxygen binding to cytochrome c oxidase and are also capable of binding to and inhibiting the enzymatic activity of carbonic anhydrase. Thus, in some embodiments an oxygen antagonist inhibits or reduces the amount of cellular respiration occurring in the cells, for instance, by binding sites on cytochrome c oxidase that would otherwise bind to oxygen. Cytochrome c oxidase specifically binds oxygen and then converts it to water. In some embodiments, such binding to cytochrome c oxidase is preferably releasable and reversible binding (e.g., has an in vitro dissociation constant, K_{d}, of at least 10⁻², 10⁻³, or 10⁻⁴ M, and has an in vitro dissociation constant, K_{d}, not greater than 10⁻⁶, 10⁻⁷, 10⁻⁸, 10⁻⁹, 10⁻¹⁰, or 10⁻¹¹ M). In some embodiments, an oxygen antagonist is evaluated by measuring ATP and/or carbon dioxide output.

The term “effective amount” means an amount that can achieve the stated result. In certain methods of the invention, an “effective amount” is, for example, an amount that induces apnea in the biological matter in need of becoming apneic. In additional embodiments, an “effective amount” may refer to an amount that increases the survivability of an organism or other biological matter, i.e., live longer than they would with having been exposed to an effective amount of a compound of the invention.

It will be understood that when inducing stasis in a tissue or organ, an effective amount is one that induces stasis in the tissue or organ as determined by the collective amount of cellular respiration of the tissue or organ. Accordingly, for example, if the level of oxygen consumption by a heart (collectively with respect to cells of the heart) is decreased at least about 2-fold (i.e., 50%) after exposure to a particular amount of a certain oxygen antagonist or other active stasis compound, it will be understood that that was an effective amount to induce stasis in the heart. Similarly, an effective amount of an agent that induces stasis in an organism is one that is evaluated with respect to the collective or aggregate level of a particular parameter of stasis. It will be also understood that when inducing stasis in an organism, an effective amount is one that induces stasis generally of the whole organism, unless a particular part of the organism was targeted. In addition, it is understood that an effective amount may be an amount sufficient to induce stasis by itself, or it may be an amount sufficient to induce stasis in combination with another agent or stimuli, e.g., another active compound, an injury, or a disease state.

The concept of an effective amount of a particular compound is related, in some embodiments, to how much utilizable oxygen there is available to the biological matter. Generally, stasis can be induced when there is about 100,000 ppm or less of oxygen in the absence of any oxygen antagonist (room air has about 210,000 ppm oxygen). The oxygen antagonist serves to alter how much oxygen is effectively available. At concentration of 10 ppm of oxygen, suspended animation is induced. Thus, while the actual concentration of oxygen that biological matter is exposed to may be higher, even much higher, than 10 ppm, stasis can be induced because of the competitive effect of an oxygen antagonist with oxygen for binding to essential oxygen metabolizing proteins in the biological matter. In other words, an effective amount of an oxygen antagonist reduces the effective oxygen concentration to a point where the oxygen that is present cannot be used. This will happen when the amount of an oxygen antagonist reduces the effective oxygen concentration below the Kₘ of oxygen binding to essential oxygen metabolizing proteins (i.e., comparable to 10 ppm of oxygen). Accordingly, in some embodiments, an oxygen antagonist reduces the effective concentration of oxygen by about 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, 1000-, 1100-, 1200-, 1300-, 1400-, 1500-, 1600-, 1700-, 1800-, 1900-, 2000-, 2100-, 2200-, 2300-, 2400-, 2500-, 2600-, 2700-, 2800-, 2900-, 3000-, 3100-, 3200-, 3300, 3400-, 3500-, 3600-, 3700-, 3800-, 3900-, 4000-, 4100-, 4200-, 4300-, 4400-, 4500-, 5000-, 6000-, 7000-, 8000-, 9000-, or 10000-fold or more, or any range derivable therein. Alternatively, it is contemplated that embodiments of the invention may be discussed in terms of a reduction in effective oxygen concentration as about, at least about, or at most about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein. It is understood that this is another way of indicating a decrease in cellular respiration.

Furthermore, in some embodiments, stasis can be measured indirectly by a drop in core body temperature of an organism. It is contemplated that a reduction in core body temperature of about, at least about, or at most about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50° F. or more, or any range derivable therein may be observed in methods of the invention. In some embodiments of the invention, hypothermia can be induced, such as moderate hypothermia (at least 10° F. reduction) or severe hypothermia (at least 20° F. reduction).

Moreover, the effective amount can be expressed as a concentration with or without a qualification on length of time of exposure. In some embodiments, it is generally contemplated that to induce stasis or achieve other stated goals of the invention, the biological matter is exposed to an active compound for about, at least about, or at most about 5, 10, 15, 20, 25, 30, 35, 40, 45, 50, 55, 60 seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein. It is further contemplated that the amount of time may be indefinite, depending on the reason or purpose for administering the active compound. Thereafter, biological matter may continue to be exposed to an active compound, or, in other embodiments of the invention, the biological matter may no longer be exposed to the active compound. This latter step can be achieved either by removing or effectively removing the active compound from the presence of the biological matter in which stasis was desired, or the biological matter may be removed from an environment containing the active compound. Additionally, matter may be exposed to or provided with any active compound continuously (a period of time without a break in exposure), intermittently (exposure on multiple occasions), or on a periodic basis (exposure on multiple occasions on a regular basis). The dosages of the active compound on these different bases may the same or they may vary. In certain embodiments, an active compound is provided periodically by providing or exposing biological matter to an active compound 1, 2, 3, 4, 5, 6, 7, 8, 9, or 10 times every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, or any range derivable therein.

Furthermore, in some embodiments of the invention, biological matter is exposed to or provided with an active compound for a sustained period of time, where “sustained” means a period of time of at least about 2 hours. In other embodiments, biological matter may be exposed to or provided with an active compound on a sustained basis for more than a single day. In such circumstances, the biological matter is provided with an active compound on a continuously sustained basis. In certain embodiments, biological matter may be exposed to or provided with an active compound for about 2, 3, 4, 5, 6, 7, 8, 9, 10, 11, 12 or more hours (or any range derivable therein) for 2, 3, 4, 5, 6, 7 days, and/or 1, 2, 3, 4, 5 weeks, and/or 1, 2, 3, 4, 5, 6, 7, 8, 9, 10, 11, 12 months, and/or 1, 2, 3, 4, 5, 6, 7, 8, 9, 10, 11, 12, 13, 14, 15, 16, 17, 18, 19, 20 or more years (or any range derivable therein) continuously, intermittently (exposure on multiple occasions), or on a periodic basis (exposure on a recurring regular basis).

In some embodiments, biological matter may be exposed to or provided with an active compound at least before and during; before, during, and after; during and after; or solely after a particular injury, trauma, or treatment (for instance, surgery), adverse condition or other relevant event or situation. This exposure may or may not be sustained.

The dosages of the active compound on these different bases may the same or they may vary.

Moreover, in certain embodiments, an active compound may be provided on a continuously sustained basis at level that is considered “low,” meaning a level that is less than the amount that causes metabolic flexibility such as seen with drop in CBT, heart rate, or CO₂ or O₂ consumption or production.

In certain embodiments, biological matter is exposed or provided an active compound, such as a metabolic agent, in an amount that exceeds what was previously understood to be the maximum tolerated dose before adverse physiological ramifications such as apnea (“period of time during which breathing is markedly reduced such that the subject takes 10% or fewer number of breaths”), lack of observable skeletal muscle movement, dystonia, and/or hyperactivity. Such an amount may be particularly relevant to increasing survivability in some embodiments of the invention, for instance, to increase the chances of surviving adverse conditions, such as those that would induce death from hemorrhagic shock.

A physiological state can be induced by active compounds of the present invention which enhances survivability in an organism in need of survivability enhancement and comprises a set of observable physiological changes in response to an effective dose of an active compound, said changes may comprise one, more or all of hyperpnea, apnea and the concomitant or subsequent loss of neuromuscular tone or voluntary control of movement with continued heartbeat. A transient and measurable change in arterial blood color may also be observed. Hyperpnea refers to rapid, shallow breathing. Apnea refers to a cessation of breathing or the reduction as described above.

In certain embodiments, the subject becomes apneic, which is marked by a cessation in breathing and then an apneic breath after a short period of time. In rats, this occurs after approximately 20 seconds. Thus, it is contemplated that a subject induced into apnea may exhibit 0, 1, 2, 3, 4, 5, 6, 7, 8, 9, 10% the number of breaths subsequent to exposure to an active compound. The subject may have an occasional breath, which may be considered an apneic breath, thereafter, In certain embodiments of the invention, apnea continues until the subject is no longer exposed to the active compound.

In some embodiments of the invention, an effective amount may be expressed as LD₅₀, which refers to the “median lethal dose,” which means the dose that is administered that kills half the population of animals (causes 50% mortality). Moreover, in further embodiments, an effective amount may be independent of the weight of the biological matter (“weight independent”). In rodents and humans, for example, the LD₅₀ of H₂S gas is approximately 700 ppm before adverse physiological effects occur, Moreover, in some embodiments of the invention, increasing survivability refers generally to living longer, which is an embodiment of the invention.

The present invention also concerns methods for inducing apnea in an organism comprising administering to the organism an effective amount of an active compound. In certain embodiments, the organism also does not exhibit any skeletal muscle movement as a result of the active compound. It is specifically contemplated that the organism may be mammal, including a human. In other embodiments, an effective amount exceeds what is considered a lethal concentration. In further embodiments, the concentration may be a lethal amount though the exposure time may be about, at least about, or at most about 10, 15, 20, 25, 30, 35, 40, 45, 50, 55, 60 seconds, 1, 2, 3, 4, 5 minutes or more (or any range derivable therein or any other time period specified in this disclosure). In particular embodiments, a mammal is exposed to at least about 600 ppm of an active gas compound, such as H₂S.

Additionally, in certain embodiments, there is a step of identifying an animal in need of treatment. In other embodiments, there is a step of observing apnea in the organism. In even further embodiments, methods involving obtaining a blood sample from the organism and/or evaluating the color of the organism's blood. It has been observed that exposure to H₂S changes the color of blood from a mammal; it goes from bright red to a darker, red wine color and then to brick red. Evaluating the color may be done visually without any instruments or machines, while in other embodiments, an instrument may be used, such as a spectrophotometer. Furthermore, a blood sample may be obtained from an organism and other types of analysis may be done on it. Alternatively, a blood sample may not be needed and instead, blood may be evaluated without the sample. For instance, a modified pulse-oximeter that shines IR or visible light through the finger may be employed to monitor color changes in the blood.

In certain embodiments, biological matter is exposed to an effective amount of an active compound that does not lead to stasis or pre-stasis. In some embodiments, there may be no evidence of a reduction in oxygen consumption or carbon dioxide production while the active compound is present.

In additional embodiments, an organism may be exposed to the active compound while sleeping. Moreover, as discussed above, the exposure may be regular, such as daily (meaning exposure at least once a day).

It is specifically contemplated that in some embodiments an active compound is provided to a subject by nebulizer. This may be applied with any embodiment of the invention. In certain cases, the nebulizer is used for the treatment of hemorrhagic shock. In further embodiments, the active compound is provided as a single dose to the subject. In specific cases, a single dose or multiple doses is one that would induce apnea in a subject. In some embodiments, a subject is given at least about 1,000, 2,000, 3,000, 4,000, 5,000, 6,000, 7,000, 8,000, 9,000, 10,000, 11,000, 12,000 or more ppm H₂S gas. The exposure time may be any of the times discussed herein, including about or about at most 10, 9, 8, 7, 6, 5, 4, 3, 2, 1, 0.5, 0.1 minutes or less (or any range derivable therein).

In further embodiments, after exposure to an active compound the metabolic rate of biologic matter may change. In certain embodiments, the RQ ratio (CO₂ production/O₂ consumption) of the biological matter changes after exposure to an active compound. This may occur after an initial exposure or repeated exposure or after an acute exposure. In some embodiments, the RQ ratio decreases after exposure. The decrease may be a decrease of about, at least about or at most about 5, 10, 15, 20, 25, 30, 35, 40, 45, 50, 55, 60, 65, 70, 75, 80% or more, or any range derivable therein. The decrease may be a result of O₂ consumption increasing of CO₂ production decreasing in relation to O₂ consumption.

In some embodiments, no other physiological change is observed in biological matter exposed to the active compound except that its RQ ratio changes after the exposure. Therefore, in some embodiments of the invention, methods involve measuring an RQ ratio in a subject. This may occur before and/or after exposure to the active compound.

Therefore, in some embodiments of the invention, stasis is induced, and a further step in methods of the invention is to maintain the relevant biological matter in a state of stasis. This can be accomplished by continuing to expose the biological matter to an active compound and/or exposing the biological matter to a nonphysiological temperature or another active compound. Alternatively, the biological matter may be placed in a preservation agent or solution, or be exposed to normoxic or hypoxic conditions. It is contemplated that biological matter may be maintained in stasis for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein. Moreover, it is contemplated that in addition to or instead of changing the temperature, other changes in the environment may be implemented such as a change in pressure or to effect a cryoprotectant or cryopreservation environment (e.g., one containing glycerol).

It will be appreciated that “stasis” with respect to a whole animal and “stasis” with respect to cells or tissues may require different lengths of time in stasis. Thus, with respect to human subjects, e.g., subjects undergoing a surgical treatment, treatment for malignant hyperthermia, or trauma victim, a time of stasis of up to 12, 18, or 24 hours is generally contemplated. With respect to non-human animal subjects, e.g. non-human animals shipped or stored for commercial purposes, stasis for a period of 2 or 4 days, 2 or 4 weeks, or longer is contemplated.

The term “expose” is used according to its ordinary meaning to indicate that biological matter is subjected to an active compound. This can be achieved in some embodiments by contacting biological matter with an oxygen antagonist or active compound. In other embodiments, this is achieved by contacting the biological matter with an active compound, which may or may not be an oxygen antagonist. In the case of in vivo cells, tissues, or organs, “expose” may further mean “to lay open” these materials so that it can be contacted with an active compound. This can be done, for example, surgically. Exposing biological matter to an active compound can be by incubation in or with (includes immersion) the antagonist, perfusion or infusion with the antagonist, injection of biological matter with an active compound, or applying an active compound to the biological matter. In addition, if stasis of the entire organism is desirable, inhalation or ingestion of the active compound, or any other route of pharmaceutical administration is contemplated for use with active compounds. Furthermore, the term “provide” is used according to its ordinary and plain meaning to mean “to supply.” It is contemplated that a compound may be provided to biological matter in one form and be converted by chemical reaction to its form as an active compound. The term “provide” encompasses the term “expose” in the context of the term “effective amount,” according to the present invention.

In some embodiments, an effective amount is characterized as a sublethal dose of the active compound. In the context of inducing stasis of cells, tissues, or organs (not the whole organism), a “sublethal dose” means a single administration of the oxygen antagonist or active compound that is less than half of the amount of the oxygen antagonist or active compound that would cause at least a majority of cells in a biological matter to die within 24 hours of the administration. If stasis of the entire organism is desired, then a “sublethal dose” means a single administration of the oxygen antagonist or active compound that is less than half of the amount of the oxygen antagonist that would cause the organism to die within 24 hours of the administration. In other embodiments, an effective amount is characterized as a near-lethal dose of the oxygen antagonist or active compound. Similarly, in the context of inducing stasis of cells, tissues, or organs (not the whole organism), a “near lethal dose” means a single administration of the oxygen antagonist or active compound that is within 25% of the amount of the inhibitor that would cause at least a majority of cell(s) to die within 24 hours of the administration. If stasis of the entire organism is desired, then a “near lethal dose” means a single administration of the oxygen antagonist or active compound that is within 25% of the amount of the inhibitor that would cause the organism to die within 24 hours of the administration. In some embodiments a sublethal dose is administered by administering a predetermined amount of the oxygen antagonist or active compound to the biological material. It is specifically contemplated that this may be implemented with respect to any active compound.

Furthermore, it is contemplated that in some embodiments an effective amount is characterized as a supralethal dose of the active compound. In the context of inducing stasis of cells, tissues, or organs (not the whole organism), a “supra-lethal dose” means a single administration of an active compound that is at least 1.5 times (1.5×) the amount of the active compound that would cause at least a majority of cells in a biological matter to die within 24 hours of the administration. If stasis of the entire organism is desired, then a “supra-lethal dose” means a single administration of the active compound that is at least 1.5 times the amount of the active compound that would cause the organism to die within 24 hours of the administration. It is specifically contemplated that the supra-lethal dose can be about, at least about, or at most about 1.5×, 2×, 3×, 4×, 5×, 10×, 20×, 30×, 40×, 50×, 60×, 70×, 80×, 90×, 100×, 150×, 200×, 250×, 300×, 400×, 500×, 600×, 700×, 800×, 900×, 1000×, 1100×, 1200×, 1300×, 1400×, 1500×, 1600×, 1700×, 1800×, 1900×, 2000×, 3000×, 4000×, 5000×, 6000×, 7000×, 8000×, 9000×, 10,000× or more, or any range derivable therein, the amount of the active compound that would cause at least a majority of cells in a biological matter (or the entire organism) to die within 24 hours of the administration.

The amount of the active compound that is provided to biological matter can be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg, mg/kg, or mg/m2, or any range derivable therein. Alternatively, the amount may be expressed as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mM or M, or any range derivable therein.

In some embodiments an effective amount is administered by monitoring, alone or in combination, the amount of active compound administered, monitoring the duration of administration of the active compound, monitoring a physiological response (e.g., pulse, respiration, pain response, movement or motility, metabolic parameters such as cellular energy production or redox state, etc.) of the biological material to the administration of the active compound and reducing, interrupting or ceasing administration of the compound(s) when a predetermined floor or ceiling for a change in that response is measured, etc. Moreover, these steps can be employed additionally in any method of the invention.

Tissue in a state of stasis or that has undergone stasis can be used in a number of applications. They can be used, for example, for transfusion or transplantation (therapeutic applications, including organ transplants); for research purposes; for screening assays to identify, characterize, or manufacture other compounds that induce stasis; for testing a sample from which the tissue was obtained (diagnostic applications); for preserving or preventing damage to the tissue that will be placed back into the organism from which they were derived (preventative applications); and for preserving or preventing damage to them during transport or storage. Details of such applications and other uses are described below. The term “isolated tissue” means that the tissue is not located within an organism. In some embodiments, the tissue is all or part of an organ. The terms “tissue” and “organ” are used according to their ordinary and plain meanings. Though tissue is composed of cells, it will be understood that the term “tissue” refers to an aggregate of similar cells forming a definite kind of structural material. Moreover, an organ is a particular type of tissue.

The present invention concerns methods for inducing stasis in isolated tissue comprising: a) identifying the tissue in which stasis is desired; and, b) exposing the tissue to an effective amount of an oxygen antagonist to induce stasis.

The present invention also provides methods, compositions, and apparati for enhancing survivability of and/or reducing damage to biological matter under adverse conditions by reducing metabolic demand, oxygen requirements, temperature, or any combination thereof in the biological matter of interest. In some embodiments of the invention, survivability of biological matter is enhanced by providing it with an effective amount of a protective metabolic agent. The agent enhances survivability by preventing or reducing damage to the biological matter, preventing all or part of the matter from dying or senescing, and/or extending the lifespan of all or part of the biological matter, relative to biological matter not exposed to the agent, Alternatively, in some embodiments the agent prolongs survival of tissue and/or an organism that would otherwise not survive without the agent.

It is contemplated that a “protective metabolic agent” is a substance or compound capable of reversibly altering the metabolism of biological matter that is exposed to or contacted with it and that promotes or enhances the survivability of the biological matter.

In certain embodiments, the protective metabolic agent induces stasis in the treated biological matter; while, in other embodiments, the protective metabolic agent does not directly itself induce stasis in the treated biological matter. Protective metabolic agents, and other active compounds, may enhance survivability and/or reduce damage to biological matter without inducing stasis in the biological matter per se, but rather by reducing cellular respiration and corresponding metabolic activity to a degree that is less than about a fifty percent decrease in oxygen consumption or carbon dioxide production. Additionally, such compounds may cause the biological matter to more quickly, easily, or effectively enter into or achieve stasis in response to an injury or disease state, e.g., by inducing the biological matter to achieve a state of pre-stasis.

Survivability includes survivability when the matter is under adverse conditions—that is, conditions under which there can be adverse and nonreversible damage or injury to biological matter. Adverse conditions can include, but are not limited to, when oxygen concentrations are reduced in the environment (hypoxia or anoxia, such as at high altitudes or under water); when the biological matter is incapable of receiving that oxygen (such as during ischemia), which can be caused by i) reduced blood flow to organs (e.g., heart, brain, and/or kidneys) as a result of blood vessel occlusion (e.g., myocardial infarction, and/or stroke), ii) extracorporeal blood shunting as occurs during heart/lung bypass surgery (e.g., “pumphead syndrome” in which heart or brain tissue is damaged as a result of cardiopulmonary bypass), or iii) as a result of blood loss due to trauma (e.g., hemorrhagic shock or surgery); hypothermia, where the biological material is subjected to sub-physiological temperatures, due to exposure to cold environment or a state of low temperature of the biological material, such that it is unable to maintain adequate oxygenation of the biological materials; hyperthermia, whereby temperatures where the biological material is subjected to supra-physiological temperatures, due to exposure to hot environment or a state of high temperature of the biological material such as by a malignant fever; conditions of excess heavy metals, such as iron disorders (genetic as well as environmental) such as hemochromatosis, acquired iron overload, sickle-cell anemia, juvenile hemochromatosis African siderosis, thalassemia, porphyria cutanea tarda, sideroblastic anemia, iron-deficiency anemia and anemia of chronic disease. It is contemplated that a protective metabolic agent is an oxygen antagonist in certain embodiments of the invention. It is also contemplated that in certain other embodiments, an oxygen antagonist is not a protective metabolic agent. In other embodiments of the invention, one or more compounds may be used to increase or enhance survivability of biological matter; reversibly inhibit the metabolism and/or activity of biological matter; reduce the oxygen requirement of biological matter; reduce or prevent damage to biological matter under adverse conditions; prevent or reduce damage or injury to biological matter; prevent aging or senescence of biological matter; and, provide a therapeutic benefit as described throughout the application with respect to oxygen antagonists. It is contemplated that embodiments relating to inducing stasis are applicable to these other embodiments as well. Therefore, any embodiment discussed with respect to stasis may be implemented with respect to these other embodiments.

An agent used for inducing stasis or any of these other embodiments may lead or provide their desired effect(s), in some embodiments, only when they are in the context of the biological matter (i.e., have no lasting effect) and/or they may provide for these effect(s) for more than 24 hours after the biological matter is no longer exposed to it. Moreover, this can also be the case when a combination of active compounds is used.

In certain embodiments, biological matter is exposed to an amount of an active compound that reduces the rate or amount of carbon dioxide production by the biological matter at least 2-fold, but also by about, at least about, or at most about 3-, 4-, 5-, 6-, 7-, 8-, 9-, 10-, 15-, 20-, 25-, 30-, 35-, 40-, 45-, 50-, 100-, 200-, 300-, 400-, 500-fold of more, or any range derivable therein. Alternatively, it is contemplated that embodiments of the invention may be discussed in terms of a reduction in the rate or amount of carbon dioxide production by the biological matter as about, at least about, or at most about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein. In still further embodiments, biological matter is exposed to an amount of an active compound that reduces the rate or amount of oxygen consumption by the biological matter at least 2-fold, but also by about, at least about or at most about 3-, 4-, 5-, 6-, 7-, 8-, 9-, 10-, 15-, 20-, 25-, 30-, 35-, 40-, 45-, 50-, 100-, 200-, 300-, 400-, 500-fold of more, or any range derivable therein. Alternatively, it is contemplated that embodiments of the invention may be discussed in terms of a reduction in the rate or amount of oxygen consumption by the biological matter as about, at least about, or at most about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein. In still further embodiments, biological matter is exposed to an amount of an active compound that decreases movement or motility by at least 10%, but also by about, at least about, or at most about 15, 20, 25, 30, 35, 40, 45, 50, 55, 60, 65, 70, 75, 80, 85, 90, 95, 99, or 100%, or any range derivable therein. As with other embodiments, these characteristics and parameters are in the context of whichever biological matter is induced into a state of stasis. Thus, if stasis is induced in an organism's heart, these parameters would be evaluated for the heart, and not the whole organism. In the context of organisms, a reduction in oxygen consumption on the order of roughly 8-fold is a kind of stasis referred to as “hibernation.” Moreover, it will be understood in this application that a reduction in oxygen consumption on the order of around 1000-fold can be considered “suspended animation.” It will be understood that embodiments of the invention concerning stasis can be achieved at the level of hibernation or suspended animation, if appropriate. It is understood that a “-fold reduction” is relative to the reduced amount; for example, if a non-hibernating animal consumes 800 units of oxygen, the hibernating animal consumes 100 units of oxygen.

Additionally, in some embodiments of the invention, methods are provided for reducing cellular respiration, which may or may not be as high as that needed to reach stasis. A reduction in oxygen consumption by about, at least about, or at most about 1, 2, 5, 10, 15, 20, 25, 30, 35, 40, 45, 50, 55, 60, 65, 70, 75, 80, 85, 90, 95, or 100% is provided in methods of the invention. This can also be expressed and assessed in terms of any cellular respiration indicator.

It is contemplated that biological matter may be exposed to one or more active compounds more than one time. It is contemplated that biological matter may be exposed to one or more active compounds 1, 2, 3, 4, 5, 6, 7, 8, 9, 10 or more times, meaning when a biological matter is exposed multiple times that there are periods of respite (with respect to exposure to the active compound) in between.

It is also contemplated that an active compound may be administered before, during, after, or any combination thereof, in relation to the onset or progression of an injurious insult or disease condition. In certain embodiments, pre-treatment of biological matter to an active compound is sufficient to enhance survivability and/or reduce damage from an injurious or disease insult. Pre-treatment is defined as exposure of the biological matter to the active compound before the onset or detection of the injurious or disease insult. Pre-treatment can be followed by termination of exposure at or near the onset of the insult or continued exposure after the onset of insult.

In certain embodiments, methods including pre-exposure to an active compound (i.e., pre-treatment) are used to treat conditions in which an injurious or disease insult is 1) scheduled or elected in advance, or 2) predicted in advance to likely occur. Examples meeting condition 1 include, but are not limited to, major surgery where blood loss may occur spontaneously or as a result of a procedure, cardiopulmonary bypass in which oxygenation of the blood may be compromised or in which vascular delivery of blood may be reduced (as in the setting of coronary artery bypass graft (CABG) surgery), or in the treatment of organ donors prior to removal of donor organs for transport and transplantation into a recipient in need of an organ transplant. Examples meeting condition 2 include, but are not limited to, medical conditions in which a risk of injury or disease progression is inherent (e.g., in the context of unstable angina, following angioplasty, bleeding aneurysms, hemorrhagic strokes, following major trauma or blood loss), or in which the risk can be diagnosed using a medical diagnostic test.

Exposure to the active compound may enhance survivability or reduce damage when exposure occurs after the onset or detection of the injurious or disease insult to achieve a therapeutic effect. Exposure to the active compound may be brief or extended. The exposure duration may be only for as long as needed to reach an indicator of stasis activity or pre-stasis (e.g., blood pCO₂, pO₂, pH, lactate, or sulfhemoglobin levels, or body temperature), or it may be longer. In certain embodiments, exposure occurs following traumatic injury to an organism and is used to induce stasis or pre-stasis in the entire organism or injured tissue therein, so as to prevent or minimize damage, e.g., ischemic and reperfusion injury prior to, during, and/or following treatment.

In one embodiment, the present invention includes a method of protecting a mammal from suffering cellular damage from a surgery, comprising providing to the mammal an amount of hydrogen sulfide or other active compound sufficient to induce the mammal to enter pre-stasis prior to the surgery. The surgery may be elective, planned, or emergency surgery, such as, e.g., cardiopulmonary surgery. The hydrogen sulfide may be administered by any means available in the art, including, e.g., intravenously or by inhalation. In specific embodiments, sulfur is provided to a subject intravenously.

In another embodiment, the present invention includes a method of protecting a mammal from suffering cellular damage from a disease or adverse medical condition, comprising providing to the mammal an amount of hydrogen sulfide or other active compound sufficient to induce the mammal to enter pre-stasis or stasis prior to the onset or progression of the disease or adverse medical condition. This embodiment may be used in the context of a variety of different diseases and adverse medical conditions, including, e.g., unstable angina, post-angioplasty, aneurism, hemorrhagic stroke or shock, trauma, and blood loss.

In specific embodiments, the invention concerns methods of preventing an organism, such as a mammal, from bleeding to death or suffering irreversible tissue damage as a result of bleeding by providing to the mammal an amount of hydrogen sulfide or other active compound sufficient to prevent the animal from bleeding to death. In certain additional embodiments, the organism may go into hemorrhagic shock but not die from excessive bleeding. The terms “bleeding” and “hemorrhaging” are used interchangeably to refer to any discharge of blood from a blood vessel. It includes, but is not limited to, internal and external bleeding, bleeding from an injury (which may be from an internal source, or from an external physical source such as from a gunshot, stabbing, physical trauma, etc.).

Moreover, additional embodiments of the invention concern prevention of death or irreversible tissue damage from blood loss or other lack of oxygenation to cells or tissue, such as from lack of an adequate blood supply. This may be the result of, for example, actual blood loss, or it may be from conditions or diseases that prevent cells or tissue from being perfused (e.g., reperfusion injury), that cause blockage of blood to cells or tissue, that reduce blood pressure locally or overall in an organism, that reduce the amount of oxygen is carried in the blood, or that reduces the number of oxygen carrying cells in the blood. Conditions and diseases that may be involved include, but are not limited to, blood clots and embolisms, cysts, growths, tumors, anemia (including sickle cell anemia), hemophilia, other blood clotting diseases (e.g., von Willebrand, ITP), and atherosclerosis. Such conditions and diseases also include those that create essentially hypoxic or anoxic conditions for cells or tissue in an organism because of an injury, disease, or condition.

In some cases, a sublethal collective dose or a nonlethal collective dose is administered to the biological matter. As discussed above, with respect to inducing stasis in biological matter that is not an entire organism, a “sublethal collective dose” means an amount of multiple administrations of the active compound that collectively is less than half of the amount of the active compound that would cause at least a majority of cell(s) to die within 24 hours of one of the administrations. In other embodiments, an effective amount is characterized as a near-lethal dose of the oxygen antagonist or other active compound. Likewise, a “near lethal collective dose” means an amount of multiple administrations of the oxygen antagonist or other active compound that is within 25% of the amount of the active compound that would cause at least a majority of cell(s) to die within 24 hours of the one of the administrations. Also, a “supra-lethal collective dose” means an amount of multiple administrations of the active compound that is at least 1.5 times the amount of the active compound that would cause at least a majority of cell(s) (or the entire organism) to die within 24 hours of the one of the administrations. It is contemplated that multiple doses can be administered so as to induce stasis in the whole organism. The definition for “sub-lethal collective dose,” “near-lethal collective dose” and “supra-lethal collective dose” can be extrapolated based on the individual doses discussed earlier for stasis in whole organisms.

Biological matter may be exposed to or contacted with more than one active compound. Biological matter may be exposed to at least one active compound, including 2, 3, 4, 5, 6, 7, 8, 9, 10 or more active compound, or any range derivable therein. With multiple active compounds, the term “effective amount” refers to the collective amount of active compounds. For example, the biological matter may be exposed to a first active compound and then exposed to a second active compound. Alternatively, biological matter may be exposed to more than one active compound at the same time or in an overlapping manner. Furthermore, it is contemplated that more than one active compounds may be comprised or mixed together, such as in a single composition to which biological matter is exposed. Therefore, it is contemplated that, in some embodiments, a combination of active compounds is employed in compositions, methods, and articles of manufacture of the invention.

Biological matter may be provided with or exposed to an active compound through inhalation, injection, catheterization, immersion, lavage, perfusion, topical application, absorption, adsorption, or oral administration. Moreover, biological matter may be provided with or exposed to an active compound by administration to the biological matter intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

Methods and apparatuses of the invention involve a protective agent that in some embodiments is an oxygen antagonist. In still further embodiments, the oxygen antagonist is a reducing agent. Additionally, the oxygen antagonist can be characterized as a chalcogenide compound. It will be understood that active compounds may also be protective agents.

In certain embodiments, the chalcogenide compound comprises sulfur, while in others, it comprises selenium, tellurium, or polonium. In certain embodiments, a chalcogenide compound contains one or more exposed sulfide groups. It is contemplated that this chalcogenide compounds contains 1, 2, 3, 4, 5, 6 or more exposed sulfide groups, or any range derivable therein. In particular embodiments, such a sulfide-containing compound is CS₂ (carbon disulfide).

Moreover, in some methods of the invention, stasis is induced in cell(s) by exposing the cell(s) to a reducing agent that has a chemical structure of (referred to as Formula I):

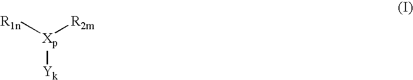

wherein X is N, O, Po, S, Se, or Te;
wherein Y is N or O;
wherein R₁ is H, C, lower alkyl, a lower alcohol, or CN;
wherein R₂ is H, C, lower alkyl, or a lower alcohol, or CN;
wherein n is 0 or 1,
wherein m is 0 or 1;
wherein k is 0, 1, 2, 3, or 4; and,
wherein p is 1 or 2.

The terms “lower alkyl” and “lower alcohol” are used according to their ordinary meanings and the symbols are the ones used to refer to chemical elements. This chemical structure will be referred to as the “reducing agent structure” and any compound having this structure will be referred to as a reducing agent structure compound. In additional embodiments, k is 0 in the reducing agent structure. Moreover, in other embodiments, the R₁ and/or R₂ groups can be an amine or lower alkyl amine. In others, R₁ and/or R₂ could be a short chain alcohol or a short chain ketone. Additionally, R₁ and R₂ may be a linear of branched chain bridge and/or the compound may be a cyclic compound. In still further embodiments, X may also be a halogen. The term “lower” is meant to refer to 1, 2, 3, 4, 5, or 6 carbon atoms, or any range derivable therein. Moreover, R₁ and/or R₂ may be other small organic groups, including, C₂-C₅ esters, amides, aldehydes, ketones, carboxylic acids, ethers, nitrites, anhydrides, halides, acyl halides, sulfides, sulfones, sulfonic acids, sulfoxides, and/or thiols. Such substitutions are clearly contemplated with respect to R₁ and/or R₂. In certain other embodiments, R₁ and/or R₂ may be short chain versions of the small organic groups discussed above. “Short chain” means 1, 2, 3, 4, 5, 6, 7, 8, 9, 10, 11, or 12 carbon molecules, or any range derivable therein.

It is contemplated that the reducing agent structure compound can be a chalcogenide compound in some cases. In certain embodiments, the chalcogenide compound has an alkyl chain with an exposed chalcogenide. In others, the chalcogenide compound has a chalcogenide that becomes exposed once it is taken up by the biological matter. In this respect, the chalcogenide compound is similar to a prodrug as an oxygen antagonist. Therefore, one or more sulfur, selenium, oxygen, tellurium, polonium, or ununhexium molecules on the compound becomes available subsequent to exposure of the biological matter to the chalcogenide compound. In this context, “available” means that the sulfur, selenide, oxygen, tellurium, polonium, or ununhexium will retain a negative charge.

In certain embodiments, the chalcogenide is a salt, preferably salts wherein the chalcogen is in a −2 oxidation state. Sulfide salts encompassed by embodiments of the invention include, but are not limited to, sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS), and the like. In like fashion, embodiments of the present invention encompass, but are not limited to, corresponding selenide and telluride salts. It is specifically contemplated that the invention includes compositions containing a chalcogenide salt (chalcogenide compound that is a salt) with a pharmaceutically acceptable carrier or prepared as a pharmaceutically acceptable formulation. In still further embodiments, the reducing agent structure compound is selected from the group consisting of H₂S, H₂Se, H₂Te, and H₂Po. In some cases, the reducing agent structure of Formula (I) has an X that is an S. In others, X is Se, or X is Te, or X is Po, or X is O. Furthermore, k in the reducing agent structure is 0 or 1 in some embodiments. In certain embodiments, the reducing agent structure compound is dimethylsulfoxide (DMSO), dimethylsulfide (DMS), carbon monoxide, methylmercaptan (CH₃SH), mercaptoethanol, thiocyanate, hydrogen cyanide, methanethiol (MeSH), or CS₂. In particular embodiments, the oxygen antagonist is H₂S, H₂Se, CS₂, MeSH, or DMS. Compounds on the order of the size of these molecules are particularly contemplated (that is, within 50% of the average of their molecular weights).

In certain embodiments, a selenium-containing compound such as H₂Se is employed. The amount of H₂Se may be in the range of 1 to 1000 parts per billion in some embodiments of the invention. It is further contemplated that any embodiment discussed in the context of a sulfur-containing compound may be implemented with a selenium-containing compound. This includes substituting one of more sulfur atoms in a sulfur-containing molecule with a corresponding selenium atom.

One aspect of the invention relates to compounds derived from mercaptoethylamine, including those represented by Formula II:

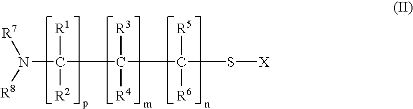

wherein n is 0, 1 or 2;
p and m are 0 or 1;
R¹, R², R³, R⁴, R⁵ and R⁶ are independently hydrogen, hydroxyl, thio, alkyl (e.g, carboxyalkyl, hydroxyalkyl, aminoalkyl) carboxyl, amino, aryl, arylalkyl, alkoxycarbonyl, alkylthiosulfonic acid, cycloalkenyl; or
R¹ or R² with R⁵ or R⁶ together are —(CH₂)_{b}—, wherein b is 2, 3, 4, 5, or 6; or
R¹ or R² and R³ or R⁴, or R³ or R⁴ and R⁵ or R⁶, together form C═C; or
R¹ and R² with R³ and R⁴, or R³ and R⁴ with R⁵ or R⁶, together form C≡C; or
R¹ and R² or R³ and R⁴ with X together form —(CHR⁹)_{c}—S—, —(CHR⁹)_{c}—SO₂—, wherein R⁹ is H or CH₃, and c is 1 or 2; and
R⁷ and R⁹ are independently hydrogen, hydroxyl, alkyl (e.g., hydroxyalkyl, arylalkyl, arylthioalkyl, arylsulfonyl, alkylthioalkyl, alkoxyalkyl, acylalkyl (e.g., (haloalkylcarbonylalkyl, aminocarbonylalkyl)), alkylsulfonyl, amino, aminoalkyl, alkylamino, alkylaminoalkyl, acyl (e.g., formyl, aminothiocarbonyl, aminocarbony, alkyliminocarbonyl, alkylthiocarbonyl), amidyl, amidino, guanidino, cycloalkylalkyl, cycloalkoxyalkyl, heteroaryloxyalkyl, nitroso, heteroaryl, hateroarylalkyl, heterocyclyl, hydrazino, hydrazide, aryloxy, heteroaryloxy;
R¹ and R² or R³ and R⁴ or R⁵ and R⁶ together are ═NH, ═NOH, ═S, ═O, —(CH₂)ₐ—, wherein a is 3, 4 or 6; and
R⁷ or R⁵ with X together form —(CHR¹⁰)_{d}—(N)ₑ—C(═Y)_{f}—(S)_{g}—, wherein R¹⁰ is H or C₁-C₂ alkyl, C₁-C₂ haloalkyl, Y is O, S or NH, d is 0, 1, or 2, e is 0 or 1, f is 0 or 1, g is 0 or 1, or P(═S)R¹¹, wherein R¹¹ is C₁-C₃ alkyl or C₁-C₃ alkoxy; or
R⁷ and R⁵ with X form ═C(R¹⁰)—, wherein R¹⁰ is thio, amino, alkylamino, or hydrazino; or
R⁷ or R⁸ with R¹ or R² or R³ or R⁴ or R⁵ or R⁶ together form a heterocyclyl ring; or
R⁷ or R⁸ with R¹ or R², or, R³ or R⁴ if p is 0, or R⁵ or R⁶ if p and m are 0, together form —N═C—; or
R⁷ and R⁸ with R¹ or R², or, R³ or R⁴ if p is 0, or R⁵ or R⁶ if p and m are 0, together form N≡C—; or
R⁷ and R⁸ together with N form a heterocyclyl substituent; or
when X is a valence bond, R⁵ or R⁶ with S form C═S; and
X is hydrogen, cyano, isocyano, phosphate, alkyl (e.g., aminoalkyl, cyanoalkyl, hydroxyalkyl, hydroxyhaloalkyl) alkenyl, alkynyl, alkylsulfonic acid, alkylthiosulfonic acid, alkylthioalkyl, arylthio, arylalkyl, alkylthio, aminoalkylthio, hydroxyalkylthio, acyl (e.g., thiocarbonyl, alkylcarbonyl, alkylthiocarbonyl, alkylaminothiocarbonyl, haloalkylcarbonyl), sulfonic acid, sulfonic alkyl ester, thiocarbonyl, thiosulfate, sulfonamido, acylthiol, acyldisulfide, alkoxythiocarbonyl, a valence bond; or X is

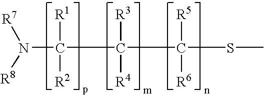

wherein R¹, R², R³, R⁴, R⁵, R⁶, R⁷ and R⁸ are as defined above.

In some embodiments, the compounds of Formula II are represented by the compounds of Formula II(a), wherein
n=0, p=1, m=1;
X is H, SO₃H, PO₃H₂ or

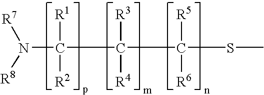

R¹ or R² with R⁵ or R⁶ together are —(CH₂)_{b}—, wherein b is 2, 3, 4, 5, or 6; or
R¹ or R² and R³ or R⁴, or R³ or R⁴ and R⁵ or R⁶, together form C═C; or
R¹ and R² with R³ and R⁴, or R³ and R⁴ with R⁵ or R⁶, together form C≡C; or
R¹ and R² or R³ and R⁴ with X together form —(CHR⁹)_{c}—S—, —(CHR⁹)_{c}—SO₂—, wherein R⁹ is H or CH₃, and c is 1 or 2; and
R⁷ and R⁸ are H; and
R¹ is C₁-C₃ alkyl and R², R³ and R⁴ are H; or
R³ is CH₃ and R¹, R² and R⁴ are H; or
R¹ and R³ together form —(CH₂)ₕ—, wherein h is 4 or 5, and R² and R⁴ are H; or
R¹ is NH₂(CH₂)ⱼ—, wherein j is 1, 2, 3, 4 or 5, and R², R³ and R⁴ are H.

In some embodiments, the compounds of Formula II are represented by the compounds of Formula II(b), wherein
n=0, p=1, m=1;
X is H, SO₃H, or PO₃H₂;
R¹, R², R³ and R⁴ are H;
R⁷ is H;
R⁸ is CH₃(CH₂)ₖ—, wherein k is 1, 2, 3, 8, 9 or 10; or
R⁸ is C₆H₅(CH₂)_{q}—, wherein q is 4 or 5 and C₆H₅ is optionally substituted with m-OCH₃ or p-OCH₃; or
R⁸ is hydroxyalkyl, which may be —CH₂CH₂OH, —CH₂CHOHCH₃, —CH(CH₂OH)CHOHCH₂OH, —CH₂CHOHCH(CH₃)CH₂C(CH₃)₃; or
R⁸ is cycloalkyloxy, which may be R¹¹—O—(CH₂)ᵤ—, wherein u is 3, 4, 5, 6, 7 or 8, and R¹¹ is (CH₂)_{w}CH—, wherein w is 4, 5 or 6, or R¹¹ is cyclohexyl, optionally substituted with one or two methyl groups; or
R⁸ is phenoxyalkyl, which may be C₆H₅—O—(CH₂)ᵥ—, wherein v is 4, 5 or 6 and C₆H₅— is optionally substituted with one or two halo, nitroso, methoxy, or C₁-C₂ alkyl; or
R⁸ is cycloalkylalkyl, which may be R¹²R¹³—, wherein R¹³ is C₄ alkyl, and R¹² is cyclohexyl or cyclohexenyl, optionally substituted with methyl or methoxy; or
R⁸ is an optionally substituted pyridyloxyalkyl or quinoyloxyalkyl; or
R⁸ is R¹⁵R¹⁶N—R¹⁷—, wherein R¹⁵ and R¹⁶ are independently H, CH₃, or H₂NC(═N)—, and R¹⁷ is C₃-C₅ alkyl; or
R⁸ is R¹⁷NH(CH₂)₃—, wherein R¹⁷ is H, CH₃ or H₂NC(═N)—.

In some embodiments, the compounds of Formula II are represented by the compounds of Formula II(c), wherein
n=0, p=1, m=1;
R¹ and X together form —(CH₂)₂ to form an thiazolidine; and
R², R³ and R⁴ are H; and
R⁷ is H; and
R⁸ is an optionally substituted pyridyloxyalkyl or quinoyloxyalkyl; or
R⁸ is R¹⁴—S—(CH₂)ₐₐ—, wherein R¹⁴ is an optionally substituted pyridyl, which may be an optionally substituted 2-pyridyl, and aa is 3, 4, 5, 6, or 7; or
R⁸ is CH₃(CH₂)_{bb}S(CH₂)₅—, wherein bb is 4 or 5.

It is contemplated that compounds used in the invention may be from any of subgroups Formula II(a), Formula II(b), or Formula II(c). Moreover, when more than one compound is employed, one or more of those compounds may or may not be from the same subgroup. Moreover, it is contemplated that in some embodiments of the invention, biological matter is provided with a precursor compound that becomes the active version of the Formula II compound by exposure to biological matter, such as by chemical or enzymatic means. In addition, the compound may be provided to the biological matter as a salt of the compound.

One aspect of the invention is directed to compounds represented by Formula III, R¹⁸SR¹⁹, wherein
R¹⁸ and R¹⁹ are independently hydrogen, alkyl, alkylthioalkyl, or SR²⁰, wherein R²⁰ is aryl, alkylaryl or alkyl.

In some embodiments, the compounds of Formula III represented by Formula III(a), wherein
R²⁰ is aryl, alkylaryl or alkyl and is substituted with one or more sodium sulfonyl (—SO₃Na) or sodium sulfonylmethyl (—CH₃SO₃Na) groups and optionally substituted with one or more halo group.

In some embodiments, the compounds of Formula III represented by Formula III(b), wherein R²⁰ is biphenyl, napthyl, benzyl, C₃-C₄ alkyl, which may be C₃-C₄ hydroxyalkyl.

In some embodiments, the compounds of Formula III represented by Formula III(c), wherein R²⁰ is biphenyl, which may be a biphen-1-yl, which may be 1′-sodium sulfonyl-biphen-1-yl, which may be 1′-sodium sulfonyl-3,3′-dichloro-biphen-1-yl.

In some embodiments, the compounds of Formula III represented by Formula III(d), wherein R²⁰ is napthyl, which may be a naphth-1-yl, which may be 7-sodium sulfonyl-naphth-1-yl.

In some embodiments, the compounds of Formula III represented by Formula III(e), wherein R²⁰ may be ortho-sodium sulfonylmethyl-benzyl.

In some embodiments, the compounds of Formula III represented by Formula III(f), wherein R²⁰ is alkyl which may be C₁-C₆ alkyl, which may be C₄, and/or which may be substituted with one or more alkylcarbonyloxy groups, which may be CH₃—CO—O—, or one or more hydroxy groups and/or sodium sulfonyl, wherein such groups include —CH₂(CHOCOCH₃)₂CH₂SO₂Na, —(CH₂)₄SO₂Na and —CH₂CHOHCH₃.

In some embodiments, the compounds of Formula III represented by Formula III(g), wherein R¹⁸ and/or R¹⁹ is alkylthioalkyl, which may be (C₁-C₆ alkyl)-thio-(C₁-C₆ alkyl), which may be CH₃(CH₂)₃S(CH₂)₂—.

In some embodiments, the compounds of Formula III represented by Formula III(h), wherein R¹⁸ and/or R¹⁹ is alkyl which may be C₁-C₆ alkyl which may be substituted with one or more sodium sulfonyl groups, which may be —(CH₂)₄SO₃Na, or substituted with one or more hydroxy groups, which may be CH₂CHOHCH₃.

It is contemplated that compounds used in the invention may be from any of subgroups Formula III(a), Formula III(b), Formula III(c), Formula III(d), Formula III(e), Formula III(f), Formula III(g), or Formula II(h). Moreover, when more than one compound is employed, one or more of those compounds may or may not be from the same subgroup. Moreover, it is contemplated that in some embodiments of the invention, biological matter is provided with a precursor compound that becomes the active version of the Formula III compound by exposure to biological matter, such as by chemical or enzymatic means. In addition, the compound may be provided to the biological matter as a salt of the compound.

A further aspect of the invention encompasses compounds represented by Formula IV:

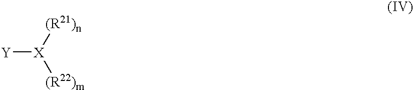

wherein:
X is N, O, P, Po, S, Se, Te, O—O, Po—Po, S—S, Se—Se, or Te—Te;
n and m are independently 0 or 1; and
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, heteroaryloxy, cyloalkyloxy, carbonyloxy, alkylcarbonyloxy, haloalkylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, —R²³R²⁴, wherein R²³ is S, SS, Po, Po—Po, Se, Se—Se, Te, or Te—Te, and R²⁴ is defined as for R²¹ herein, or Y is

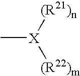

wherein X, R²¹ and R²², are as defined herein.

Moreover, it is contemplated that in some embodiments of the invention, biological matter is provided with a precursor compound that becomes the active version of the Formula I or IV compound by exposure to biological matter, such as by chemical or enzymatic means. In addition, the compound may be provided to the biological matter as a salt of the compound., in the form of a free radical, or a negatively charged, positively charged or multiply charged species. Some compounds qualify as both a Formula I and a Formula IV compound and in such cases, the use of the phrase “Formula I or Formula IV” is not intended to connote the exclusion of such compounds.

A compound identified by the structure of Formula I or Formula IV may also, in certain embodiments, be characterized as an oxygen antagonist, protective metabolic agent, or a precursor, prodrug, or salt thereof. It is further contemplated that the compound need not be characterized as such or qualify as such to be a compound used in the invention, so long as it achieves a particular method of the invention. In some other embodiments, the compound may be considered a chalcogenide compound. It is specifically contemplated that any compound identified by the structure of Formula I or Formula IV or set forth in this disclosure may be used instead of or in addition to an oxygen antagonist in methods, compositions, and apparatuses of the invention; similarly, any embodiments discussed with respect to any of structure having Formula I or Formula IV or otherwise set forth in this disclosure may be may be used instead of or in addition to an oxygen antagonist. Moreover, any compound identified by the structure of Formulas I or IV or set forth in this disclosure may be combined with any active compound described herein. It is also contemplated that any combination of such compounds may be provided or formulated together, sequentially (overlapping or nonoverlapping), and/or in an overlapping sequential manner (the administration of one compound is initiated and before that is complete, administration of another compound is initiated) in methods, compositions, and other articles of manufacture of the invention to achieve the desired effects set forth herein.

In certain embodiments, more than one compound with the structure of Formula I or Formula IV is provided. In certain embodiments, multiple different compounds with a structure from the same formula (i.e., Formula I or Formula IV) are employed, while in other embodiments, when multiple different compounds are employed, they are from different formulas.

In specific embodiments, it is contemplated that multiple active compounds are used, wherein one of the compounds is carbon dioxide (CO₂). It is contemplated that at least one other compound is also a Formula I and/or Formula IV compound in some embodiments. In certain cases, carbon dioxide is provided to biological matter in combination with H₂S or an H₂S precursor (together, sequentially, or in an overlapping sequential manner).

The amount of carbon dioxide to which the biological matter may be exposed are about, at least about, or at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30% or more, or any range derivable therein. In certain embodiments, the amount is expressed in terms of ppm, such as about, at least about, or at most about 350, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 60000, 70000, 80000, 90000, 100000, 110000, 120000, 130000, 140000, 150000, 160000, 170000, 180000, 190000, 200000, 210000, 220000, 230000, 240000, 250000, 260000, 270000, 280000, 290000, 300000 or more ppm, or any range derivable therein, as well as an molar equivalents. It is contemplated that these concentrations could apply to any other active compound in gaseous form.

In other embodiments, it is specifically contemplated that the active compound is sodium sulfide, sodium thiomethoxide, cysteamine, or tetrahydrothiopryan-4-ol. In additional embodiments, the active compound is sodium thiocyanate, cysteamine-S-phosphate sodium salt, dimethylsulfoxide, thioacetic acid, selenourea, amifostine, 2-mercapto-ethanol, thioglycolicether, sodium selenide, sodium methane sulfinate, thiourea, or dimethylsulfide. It is specifically contemplated that these compounds, or any others discussed herein including any compound with Formula I or IV, may be provided or administered to biological matter in an amount that is about, at least about, or at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 87, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000 mM or mmol/kg (of biological matter), or any range derivable therein.

It is specifically contemplated that any subset of active compounds identified by name or structure may be used in methods, compositions and articles of manufacture. It is also specifically contemplated that any subset of these compounds may be disclaimed as not constituting embodiments of the invention. The present invention also concerns pharmaceutical compositions comprising a therapeutically effective amount of one or more active compounds. It is understood that such pharmaceutical compositions are formulated in pharmaceutically acceptable compositions. For example, the composition may include a pharmaceutically acceptable diluent.

In certain embodiments, the pharmaceutical composition contains an effective dose of an active to provide when administered to a patient a Cₘₐₓ or a steady state plasma concentration of the active compound to produce a therapeutically effective benefit. In certain embodiments, the Cₘₐₓ or steady state plasma concentration to be achieved is about, at least about, or at most about 0.01, 0.1, 1, 2, 3, 4, 5, 6, 7, 8, 9, 10, 1, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 3000, 4000, 5000, 6000, 7000, 8000, 9000, 10000 μM or more, or any range derivable therein. In certain embodiments, such as with H₂S, the desired Cₘₐₓ or steady state plasma concentration is about between 10 μM to about 10 mM, or between about 100 μM to about 1 mM, or between about 200 μM to about 800 μM. Appropriate measures may be taken to consider and evaluate levels of the compound already in the blood, such as sulfur.

In certain embodiments, the pharmaceutical composition provides an effective dose of H₂S to provide when administered to a patient a Cₘₐₓ or a steady state plasma concentration of between 10 μl to 10 mM, between about 100 μM to about 1 mM, or between about 200 μM to about 800 μM. In relating dosing of hydrogen sulfide to dosing with sulfide salts, in typical embodiments, the dosing of the salt is based on administering approximately the same sulfur equivalents as the dosing of the H₂S. Appropriate measures will be taken to consider and evaluate levels of sulfur already in the blood.

In certain embodiments, the composition comprises a gaseous form of one or more of the active compounds specified above. In another embodiment, the composition comprises a salt of one or more of these compounds. In one particular embodiment, a pharmaceutical composition comprises a gaseous form of Formula I, II, III, or IV or a salt thereof. A gaseous form or salt of H₂S is specifically contemplated in some aspects of the invention. It is contemplated that the amount of gas to which biological matter is provided is about, at least about, or at most about 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 60000, 70000, 80000, 90000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000 or more ppm, or any range derivable therein. Alternatively, the effective amount of gas(es) may be expressed as about, at least about, or at most about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range derivable therein, with respect to the concentration in the air to which the biological matter is exposed. Moreover, it is contemplated that with some embodiments, the amount of gas to which biological matter is provided is about, at least about, or at most about 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 parts per billion (ppb) or any range derivable therein. In particular embodiments, the amount of hydrogen selenide provided to biological matter is on this order of magnitude.

In some embodiments of the invention, the pharmaceutical composition is a liquid. As discussed elsewhere, the composition may be a liquid with the relevant compound(s) dissolved or bubbled into the composition. In some cases, the pharmaceutical composition is a medical gas. According to the United States Food and Drug Administration, “medical gases” are those gases that are drugs within the meaning of §201(g)(1) of the Federal Food, Drug and Cosmetic Act (“the Act”) (21 U.S.C. §201(g) and pursuant to §503(b)(1)(A) of the Act (21 U.S.C. §353(b)(1)(A)) are required to be dispensed by prescription. As such, such medical gases require an appropriate FDA label. A medical gas includes at least one active compound.

The present invention further comprises apparatuses and articles of manufacture comprising packaging material and, contained within the packaging material, an active stasis compound, wherein the packaging material comprises a label that indicates that it can be used for inducing stasis in in vivo biological matter.

In some embodiments, the apparatus or article of manufacture further includes a pharmaceutically acceptable diluent. In particular other embodiments, the apparatus or article of manufacture has a buffering agent. The active compound is provided in a first sealed container and the pharmaceutically acceptable diluent is provided in a second sealed container. In other embodiments, the device or article further has instructions for mixing the active compound and the diluent. Additionally, the active compound can be reconstituted for achieving any method of the invention, such as for inducing stasis in in vivo biological matter. It is contemplated that any label would specify the result to be achieved and the use of the compound for patients in need of that result.

The present invention also concerns an article of manufacture comprising packed together: an active compound, instructions for use of the active stasis compound, comprising: (a) identifying in vivo tissue in need of stasis treatment; and (b) administering an effective amount of the active compound to the in vivo biological matter.

In further embodiments of the invention, there is an article of manufacture comprising a medical gas including an active compound and a label comprising details or use and administration for inducing stasis in a biological matter or any other method of the invention.

The present invention also concerns kits and methods of using these kits. In some embodiments, there are kits for the delivery of an active compound to a tissue site in need of stasis treatment, or any other treatment of the claimed invention, comprising: a drape adapted for forming a sealed envelope against a tissue site; a container comprising an oxygen antagonist; and an inlet in the drape, wherein the container comprising the active compound is in communication with the inlet. In certain embodiments, the kit includes an outlet in the drape wherein the outlet is in communication with a negative pressure source. In some cases, the drape comprises elastomeric material and/or has a pressure sensitive adhesive covering the periphery of the drape. The outlet may be placed in fluid communication with the negative pressure source, which may or may not be a vacuum pump. There may also be a flexible conduit communicating between the outlet and the negative pressure source. In some embodiments, the kit includes a canister, which may or may not be removable, in fluid communication between the outlet and the negative pressure source. It is contemplated that the container includes an active compound that is in gaseous communication with the inlet. In certain embodiments, the container includes an active compound that is a gas or a liquid gas. The kit may also include a vaporizer in communication between the container comprising an oxygen antagonist and the inlet. In addition, it may have a return outlet in communication with the container comprising the active compound.

In particular embodiments, the active compound in the kits is carbon monoxide, carbon dioxide, H₂Se, and/or H₂S. In certain embodiments, the tissue site for which the kit or method is applied is wounded.

Moreover, it will be generally understood that any compound discussed herein as an oxygen antagonist can be provided in prodrug form to the biological matter, meaning that the biological matter or other substances) in the environment of the biological matter alters the prodrug into its active form, that is, into an oxygen antagonist. It is contemplated that the term “precursor” covers compounds that are considered “prodrugs.”

The active compound may be or may be provided as a gas, semi-solid liquid (such as a gel or paste), liquid, or solid. It is contemplated that biological matter may be exposed to more than one such compound and/or to that compound in more than one state. Moreover, the agent may be formulated for a particular mode of administration, as is discussed herein. In certain embodiments, the agent is in pharmaceutical acceptable formulation for intravenous delivery.

In certain embodiments, the active compound is a gas. In particular embodiments, the gaseous compound includes carbon monoxide, carbon dioxide, nitrogen, sulfur, selenium, tellurium, or polonium, or a mixture thereof. Moreover, it is specifically contemplated that the compound is a chalcogenide compound as a gas. In some embodiments, the compound is in a gas mixture comprising more than one gas. The other gas(es) is a non-toxic and/or a non-reactive gas in some embodiments. In some embodiments, the other gas is a noble gas (helium, neon, argon, krypton, xenon, radon, or ununoctium), nitrogen, nitrous oxide, hydrogen, or a mixture thereof. For instance, the non-reactive gas may simply be a mixture that constitutes “room air,” which is a mixture of nitrogen, oxygen, argon and carbon dioxide, as well as trace amounts of other molecules such as neon, helium, methane, krypton, and hydrogen. The precise amounts of each varies, though a typical sample might contain about 78% nitrogen, 21% oxygen, 0.9% argon, and 0.04% carbon dioxide. It is contemplated that in the context of the present invention, “room air” is a mixture containing about 75 to about 81% nitrogen, about 18 to about 24% oxygen, about 0.7 to about 1.1% argon, and about 0.02% to about 0.06% carbon dioxide. A gaseous active compound may be first diluted with a non-toxic and/or non-reactive gas prior to administration or exposure to biological matter. Additionally or alternatively, any gaseous active compound may be mixed with room air prior to administration or exposure to biological matter or the compound may be administered or exposed to the biological matter in room air.

In some instances, the gas mixture also contains oxygen. An active compound gas is mixed with oxygen to form an oxygen gas (O₂) mixture in other embodiments of the invention. Specifically contemplated is an oxygen gas mixture in which the amount of oxygen in the oxygen gas mixture is less than the total amount of all other gas or gases in the mixture.

In some cases, the amount of an active compound is relative to the amount of oxygen, while in others, it is an absolute amount. For example, in some embodiments of the invention, the amount of oxygen is in terms of “parts per million (ppm)” which is a measure of the parts in volume of oxygen in a million parts of air at standard temperature and pressure of 20° C. and one atmosphere pressure and the balance of the gas volume is made up with carbon monoxide. In this context, the amount of the active compound to oxygen is related in terms of parts per million of oxygen balanced with the active compound. It is contemplated that the atmosphere to which the biological material is exposed or incubated may be at least 0, 50, 100, 200, 300, 400, 500, 1000, or 2000 parts per million (ppm) of oxygen balanced with the active compound and in some cases the active compound mixed with a non-toxic and/or non-reactive gas The term “environment” refers to the immediate environment of the biological matter, that is, the environment with which it is in direct contact. Thus, the biological material must be directly exposed to the active compound, and it is insufficient that a sealed tank of the active compound be in the same room as the biological matter and be considered to be incubated an “environment” according to the invention. Alternatively, the atmosphere may be expressed in terms of kPa. It is generally understood that 1 million parts=101 kPa at 1 atmosphere. In embodiments of the invention, the environment in which a biological material is incubated or exposed to is about, at least about, or at most about 0.001, 0.005, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20. 0.21, 0.22, 0.23, 0.24, 0.25, 0.26, 0.27, 0.28, 0.29, 0.30, 0.35, 0.40, 0.45, 0.50, 0.55, 0.60, 0.65, 0.70, 0.75, 0.80, 0.5, 0.90, 0.95, 1.0 kPa or more O₂, or any range derivable therein. As described above, such levels can be balanced with carbon monoxide and/or other non-toxic and/or non-reactive gas(es) Also, the atmosphere may be defined in terms of levels of the active compound in kPa units. In certain embodiments, the atmosphere is about, at least about, or at most about 1, 5, 10, 15, 20, 25, 30, 35, 40, 45, 50, 55, 60, 65, 70, 75, 80, 85, 90, 95, 101, 101.3 kPa, or any range derivable therein.

In some embodiments, the invention concerns compositions and articles of manufacture that contain one or more active compounds. In certain embodiments, a composition has one or more of these active compounds as a gas that is bubbled in it so that the composition provides the compound to the biological matter when it is exposed to the composition. Such compounds may be gels, liquids, or other semi-solid material. In certain embodiments, a solution has an oxygen antagonist as a gas bubbled through it. It is contemplated that the amount bubbled in the gas will provide the appropriate amount of the compound to biological material exposed to the solution. In certain embodiments, the amount of gas bubbled into the solution is about, at least about, or at most about 0.5, 1.0, 1.5, 2.0. 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 95, 100 times or more, or any range derivable therein, than the amount to which the biological matter is effectively provided.

Biological matter may be exposed to the gas in a closed container in some embodiments of the invention. In some cases, the closed container can maintain a particular environment or modulate the environment as is desired. The environment refers to the amount of oxygen antagonist that the biological matter is exposed and/or the temperature, gas composition, or pressure of the environment. In some cases, the biological matter is placed under a vacuum before, during, or after exposure to an active compound.

Moreover, in other cases, the biological matter is exposed to a normoxic environment after being exposed to an active compound. In certain embodiments, the present invention includes methods for inducing stasis or protecting biological matter from injury or disease that include providing an active compound to the biological matter in combination with providing another stasis-inducing active compound or environmental condition to the biological matter. Such combination treatment may occur in any order, e.g., simultaneously or sequentially.

In certain embodiments, the present invention contemplates methods of enhancing survivability of biological matter comprising: (a) providing to the biological matter an effective amount of at least one active compound; and (b) placing the biological matter under hypoxic conditions. In such methods, the hypoxic conditions may further be defined as anoxic conditions. In certain embodiments, steps (a) and (b) may be performed sequentially or simultaneously. In particular embodiments, step (b) is performed after step (a). In certain embodiments, the active compound is provided while the biological matter is subjected to hypoxic conditions. In other embodiments, the active compound ceases to be provided to the biological matters and then the biological matter is subjected to hypoxic conditions.

Any active compound, including H₂S or CO₂, is contemplated for administration before subjecting biological matter to hypoxic conditions, or during the subjection of biological matter to hypoxic conditions. In certain embodiments, only one active compound is provided to the biological matter. In particular embodiments, H₂S is provided alone or in combination with one or more active compounds. Stasis may or may not be induced during or following the provision of an effective amount of at least one compound before subjecting the biological matter to hypoxic conditions. Hypoxic conditions of any type described herein may be provided. The provision of hypoxic conditions may comprise subjecting the biological matter to about 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% O₂, or O₂-free conditions, or any range derivable therein. In particular embodiments, biological matter is subjected to 5% O₂.

The provision of an effective amount of at least one active compound and/or the time for which biological matter is placed under hypoxic or anoxic conditions may each be for any sustained period of time, as described herein. The period of time may also range anywhere from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 630, 660, 690, 720, 750, 780, 810, 840, 870, 900, 930, 960, 990, 1020, 1080, 1140, 1200, 1260, 1320, 1380, 1440, 1500, 1560, 1620, 1680, 1740, 1800, 1860 1920, 1980, 2040, 2100, 2160, 2220, 2280, 2340, or 2400 minutes or more, or any derivable range therein. In certain embodiments, the biological matter is provided with at least one active compound for a period of about 60 minutes or less. In certain embodiments, the biological matter is provided with at least one active compound for a period of about 20 minutes or less. In certain embodiments, the biological matter is exposed to hypoxic conditions of about 5% O₂ for about 360 minutes (6.5 hours) or less. In certain embodiments, the biological matter is exposed to hypoxic conditions of about 5% O₂ for about 60 minutes (1 hour) or less. In certain embodiments, the biological matter is exposed to hypoxic conditions of about 3% O₂ for about 240 minutes (4 hours) or less. In certain embodiments, the biological matter is exposed to anoxic conditions for a period of about 2400 minutes (40 hours) or less.

In certain embodiments, at least one active compound is provided to biological matter, and the biological matter is subsequently placed under hypoxic conditions. Hypoxic conditions of any type described herein may be provided. In certain embodiments, hypoxic conditions may comprise subjecting the biological matter to at most about or about 20.8, 20, 19, 18, 17, 16, 15, 14, 13, 12, 11, 10, 9, 8, 7, 6, 5, 4, 3, 2, 1, 0.5, or 0% O₂. In certain embodiments, hypoxic conditions of about 5% O₂ are provided to the biological matter, In further embodiments, following the subjection of biological matter to hypoxic conditions, the biological matter is sequentially exposed to increasingly hypoxic conditions: for example, about 5% O₂ followed by about 4% O₂, 3% O₂, 2% O₂, 1% O₂, or O₂-free conditions, or any sequential combination of such conditions. In certain embodiments, such administration of an active compound followed by (either simultaneously or sequentially) increasingly hypoxic conditions are administered before, during, or after (or any combination thereof) an injury or trauma or the onset or progression of disease. In such embodiments, the biological matter may be protected. In certain embodiments, H₂S is administered to hemorrhaging biological matter and/or biological matter suffering from hemorrhagic shock, and then sequentially, increasingly hypoxic conditions are administered (e.g., about 5% O₂, then about 4% O₂).

In certain embodiments, an active compound is provided to biological matter, wherein the active compound is a gas, and the biological matter is subsequently placed under hypoxic conditions. The gaseous active compound may be, for example, H₂S. In certain embodiments, H₂S is provided to biological matter, followed by the sequential or simultaneous provision of increasingly hypoxic conditions to the biological matter.

When providing one or more gaseous active compounds to biological matter and then simultaneously or sequentially providing hypoxic conditions and/or increasingly hypoxic conditions to the biological matter, a gas blender may be used. Gas blenders permit automated administration of one or more gases to biological matter, such as a mammal. For example, a gas blender may be used to provide biological matter with H₂S, followed by 5% O₂, followed by increasingly hypoxic conditions (e.g., about 4% O₂ or about 3% O₂). Gas blenders are well-known to those of skill in the art, and are described in more detail herein.

In certain embodiments, the present invention contemplates a method for enhancing survivability of biological matter that is at risk of hemorrhagic shock, is suffering hemorrhagic shock, and/or is hemorrhaging comprising; (a) providing to the biological matter an effective amount of at least one active compound; and (b) placing the biological matter under hypoxic conditions. The method may further comprise subjecting the biological matter to increasingly hypoxic conditions. In certain embodiments, a method of first administering H₂S to hemorrhaging biological matter and/or biological matter suffering from hemorrhagic shock and then sequentially administering increasingly hypoxic conditions to the biological matter is contemplated.

The present invention also contemplates methods of: (a) providing to biological matter an effective amount of at least one active compound; and (b) placing the biological matter under hypoxic conditions wherein the metabolic activity of the biological matter is monitored. For example, the O₂ consumption by and/or CO₂ production of the biological matter may, in certain embodiments, be monitored. In certain embodiments, the provision of at least one active compound to biological matter and the simultaneous or sequential provision of hypoxic conditions (optionally including increasingly hypoxic conditions) results in decreased oxygen demand by the biological matter. In certain embodiments, the provision of at least one active compound to biological matter and the simultaneous or sequential provision of hypoxic conditions (optionally including increasingly hypoxic conditions) results in the decrease of cytochrome c oxidase activity. Assays for cytochrome c oxidase activity are well-known to those of skill in the art. See, e.g., Sigma-Aldrich (St. Louis, Mo.) Cytochrome c Oxidase Assay Kit Catalog Ref. CYTOCOX1; MitoScience (Eugene, Oreg.) Cytochrome c Oxidase Colorimeteric Assay Kit Catalog No. MS400A.

In certain embodiments, provision of at least one active compound is simultaneously or sequentially followed by the provision of hypoxic and/or increasingly hypoxic conditions that are provided before, during, and/or after an injury or trauma or the onset or progression of disease. In certain further embodiments, the biological matter is protected.

In certain embodiments, the present invention contemplates a method of protecting biological subject from an injury, the onset or progression of a disease, or death comprising: (a) providing to the subject, before the injury, the onset or progression of a disease, or death, an effective amount of at least one active compound; and (b) placing the biological matter under hypoxic conditions.

In other embodiments, the present invention contemplates methods for preventing or reducing damage to biological matter under adverse conditions, comprising: (a) providing to the biological matter an effective amount of at least one active compound; and (b) placing the biological matter under hypoxic conditions, wherein damage is prevented or reduced.

Also contemplated by the present invention are methods for reversibly inhibiting metabolism in an organism comprising: (a) providing to the biological matter an effective amount of at least one active compound; and (b) placing the biological matter under hypoxic conditions.

In other embodiments, the present invention contemplates a method of preventing an organism from bleeding to death comprising: (a) providing to the biological matter an effective amount of at least one active compound; and (b) placing the biological matter under hypoxic conditions, wherein death is prevented.

Any embodiment discussed with respect to methods of enhancing survivability of biological matter comprising providing to the biological matter an effective amount of at least one active compound and placing the biological matter under hypoxic conditions may be implemented with respect to any other embodiment described herein.

Moreover, in other embodiments, the environment containing the biological matter cycles at least once to a different amount or concentration of the active compound, wherein the difference in amount or concentration is by at least one percentage difference. The environment may cycle back and forth between one or more amounts or concentrations of the active compound, or it may gradually increase or decrease the amount or concentrations of an that compound. In some cases, the different amount or concentration is between about 0 and 99.9% of the amount or concentration of the active compound to which the biological matter was initially exposed. It is contemplated that the difference in amount and/or concentration is about, at least about, or at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein.

Methods of the invention can also include a step of subjecting biological matter to a controlled temperature environment. In certain embodiments, the biological matter is exposed to a temperature that is a “nonphysiological temperature environment,” which refers to a temperature in which the biological matter cannot live in for more than 96 hours. The controlled temperature environment can have a temperature of about, at least about, or at most about −210, −200, −190, −180, −170, −160, −150, −140, −130, −120, −110, −100, −90, −80, −70, −60, −50, −40, −30, −20, −10, −5, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200° C. or more, or any range derivable therein. Biological matter may also be exposed to an active compound at room temperature, which means a temperature between about 20° C. and about 25° C. Furthermore, it is contemplated the biological matter achieves a core temperature of any amount or range of amounts discussed.

It is contemplated that the biological matter can be subjected to a nonphysiological temperature environment or a controlled temperature environment before, during or after exposure to the active compound(s). Furthermore, in some embodiments, the biological matter is subjected to a nonphysiological temperature environment or a controlled temperature environment for a period of time between about one minute and about one year. The amount of time may be about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein. Moreover, there may also be a step of increasing the ambient temperature relative to the reduced temperature.

Moreover, it is contemplated that the temperature may be altered or cycled during the process in which temperature is controlled. In some embodiments, the temperature of the biological matter may first be reduced before it is placed in the environment that has the active compound, while in others, the biological matter may be cooled by placing it in the environment with the active compound, that is below the temperature of the biological matter. The biological matter and/or environment may be cooled or heated gradually, such that the temperature of the biological matter or environment starts at one temperature but then reaches another temperature.

Methods of the invention can also include a step of subjecting biological matter to a controlled pressure environment. In certain embodiments, the biological matter is exposed to pressure that is lower than the pressure under which the organism is typically under. In certain embodiments, the biological matter is subjected to a “nonphysiological pressure environment,” which refers to a pressure under which the biological matter cannot live under for more than 96 hours. The controlled pressure environment can have a pressure of about, at least about, or at most 10⁻¹⁴, 10⁻¹³, 10⁻¹², 10⁻¹¹, 10⁻¹⁰, 10⁻⁹, 10⁻⁸, 10⁻⁷, 10⁻⁶, 10⁻⁵, 10⁻⁴, 10⁻³, 10⁻², 10⁻¹, 0.2, 0.3, 0.4 or 0.5 atm or more, or any range derivable therein.

It is contemplated that the biological matter can be subjected to a nonphysiological pressure environment or a controlled pressure environment before, during or after exposure to the active compound(s). Furthermore, in some embodiments, the biological matter is subjected to a nonphysiological pressure environment or a controlled pressure environment for a period of time between about one minute and about one year. The amount of time may be about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein.

Moreover, it is contemplated that the pressure may be altered or cycled during the process in which pressure is controlled. In some embodiments, the pressure to which the biological matter is exposed may first be reduced before it is placed in the environment that has the active compound, while in others, the biological matter placed under pressure after exposure to an active compound. The pressure may be reduced gradually, such that the pressure of the environment starts at one pressure but then reaches another pressure within 10, 20, 30, 40, 50, 60 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, 7 days or more, and any combination or range derivable therein. In certain embodiments, methods include modulating environmental oxygen levels or removing the biological material from an environment having oxygen. Operationally, exposing biological material to an environment in which oxygen is diminished or absent may mimic exposure of the biological material to an oxygen antagonist. It is contemplated that in some embodiments of the invention, biological matter is exposed to or provided with an active compound under conditions in which the environment of the biological matter is hypoxic or anoxic, as described in further detail below. This may be intentional or nonintentional. Thus, in some embodiments of the invention, biological matter is intentionally placed in an environment that is anoxic or hypoxic or in an environment that is made anoxic or hypoxic. In other embodiments, the biological matter is under such conditions as a result of an unintended situation, for example, if the biological matter is under ischemic or potentially ischemic conditions. Therefore, it is contemplated in some cases that the hypoxic or anoxic conditions would damage the matter in the absence of the active compound.

In certain methods of the invention, there also is a step of assessing the level of the oxygen antagonist and/or oxidative phosphorylation in the biological matter in which stasis was induced. Moreover, in some embodiments of the invention, there is a step of assessing the level of cellular metabolism that is generally occurring in the biological matter. In some cases, the amount of the active compound in the biological matter is measured and/or a reduction in the temperature of the biological matter is assessed. Moreover, in some methods of the invention, the extent of one or more therapeutic effects is evaluated.

In certain other embodiments, any toxicity effect on the biological matter from an active compound and/or environmental change (temperature, pressure) is monitored or controlled for. It is contemplated that toxicity can be controlled for by altering the level, amount, duration, or frequency of an active compound and/or environmental change to which the biological matter is exposed. In certain embodiments the alteration is a reduction, while in certain other embodiments, the alteration is an increase. It is contemplated that the skilled artisan is aware of a number of ways of evaluating toxicity effects in biological matter.

Other steps for methods of the invention include identifying an appropriate active compound; diagnosing the patient; taking a patient history and/or having one or more tests done on the patient prior to administering or prescribing an active compound to the patient.

Methods of the invention also concern inducing stasis in in vivo biological matter comprising incubating the biological matter with an active compound that creates hypoxic conditions for an effective amount of time for the biological matter to enter stasis.

Furthermore, other embodiments of the invention include methods of reducing oxygen demand in in vivo biological matter comprising contacting the biological matter with an effective amount of an active compound to reduce their oxygen demand. It is contemplated that oxygen demand is reduced about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range derivable therein, with respect to the amount of oxygen demand in cells of the biological matter or a representative sample of cells from the biological matter not exposed or no longer exposed to the active compound.

Other aspects of the invention concern methods for preserving in vivo biological matter comprising exposing the in vivo biological matter to an effective amount of an active compound to preserve the biological matter in vivo.

The present invention also concerns a method of delaying or reducing the effects of trauma on or in an organism comprising exposing biological matter at risk for trauma to an effective amount of an active compound.

In other aspects of the invention, there are methods for treating or preventing hemorrhagic shock in a patient comprising exposing the patient to an effective amount of an active compound. Alternatively, in some embodiments methods prevents lethality in the patient as a result of the bleeding and/or hemorrhagic shock. In such methods of preventing a patient from bleeding to death or prevent lethality in a bleeding patient, steps include exposing the patient to an effective amount of an active compound. In certain embodiments, the active compound is specifically contemplated to be a chalcogenide compound such as H₂S.

Methods for reducing heart rate in an organism are also included as part of the invention. Such methods involve contacting the biological sample or organism with an effective amount of an active compound.

Temperature regulation can be achieved in organisms by employing oxygen antagonists. In some embodiments, there is a method of treating a subject with hypothermia comprising (a) contacting the subject with an effective amount of an oxygen antagonist, and then (b) subjecting the subject to an environmental temperature above that of the subject. In other embodiments, the present invention includes a method of treating a subject with hyperthermia comprising (a) contacting the subject with an effective amount of an oxygen antagonist. In some cases, treatment of hyperthermia also includes (b) subjecting the subject to an environmental temperature that is at least about 20° C. below that of the subject. As discussed above, exposing the subject to nonphysiological or a controlled temperature environment can be used in additional embodiments. It is contemplated that this method may be achieved with active compounds generally.

Other aspects of the invention relate to a method for preventing hematologic shock in a patient comprising administering to the patient an effective amount of an active compound.

The present invention also covers reducing the oxygen requirement of biological matter, meaning that the amount of oxygen required by the biological matter to survive is reduced. This can be achieved by providing an effective amount of one or more active compounds. It is generally known how much oxygen particular biological matter require to survive, which can also be dependent on time, pressure, and temperature. In certain embodiments of the invention, the oxygen requirement of the biological matter is reduced by about at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any range derivable therein, as compared to the requirement of the biological matter in the absence of the effective amount of the active compound(s).

In cases in which biological matter is being protected from damage or further damage, it is contemplated that the biological matter may be exposed to an oxygen antagonist within about, within at least about, or within at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein, after initial damage (trauma or wound or degeneration) occurs. Thus in additional embodiments of the invention, methods include an initial assessment of any damage, trauma, a wound, or degeneration.

Other aspects of the invention concern methods for preserving one or more cells that are separate from an organism comprising contacting the cell(s) with an effective amount of an oxygen antagonist to preserve the one or more cells. In addition to the cells and cell types discussed above and elsewhere in this application, it is contemplated that shrimp embryos are specifically contemplated for use with the present invention.

In one embodiment, oxygen reduction techniques can be embodied in a kit. For example, the kit currently sold under product number 261215, available from Becton Dickinson, makes use of select techniques described here. That kit includes an anaerobic generator (e.g., a hydrogen gas generator), Palladium Catalysts, an anaerobic indicator, and a gas impermeable, sealable, “BioBag” into which the above components (together with platelets in a gas-permeable bag) are placed and sealed.

In other embodiments of the invention, methods are provided for enhancing the ability of biological matter to enter stasis in response to an injury or disease by providing an effective amount of an active compound, thereby protecting the biological matter from damage or injury, thereby enhancing survival of biological matter. Related embodiments include methods of preparing or priming biological matter for entry into stasis in response to an injury or disease by providing an effective amount of an active compound. Other related embodiments include method of inducing biological matter into pre-stasis, thereby protecting the biological matter from damage or injury. For example, treatment with an active compound at a dosage or for a time less than required to induce stasis enables the biological matter to more readily or more completely achieve a beneficial state of stasis in response to an injury or disease, while in the absence of treatment with the active compound, the biological matter would die or suffer damage or injury before it reached a protective level of stasis, e.g., a level sufficient to render the biological matter resistant to lethal hypoxia.

Certain injuries and disease states cause biological matter to reduce its metabolism and/or temperature to degrees that may not achieve stasis. For example, hypoxia, ischemia, and blood loss all reduce the amount of oxygen available and supplied to oxygen utilizing biological matter, thereby reducing oxygen utilization in cells of the biological matter, reducing energy production derived from oxidative phosphorylation, and thereby decreasing thermogenesis, leading to hypothermia. Depending on the severity or time elapsed following the onset or progression of the injurious or disease insult, “stasis” may or may not have been achieved. Treatment with an active compound lowers the threshold (i.e., the severity or duration of the insult that is needed to achieve stasis) for induction of stasis, or it may add to or synergize with the injurious or disease stimuli to induce stasis in biological matter under injurious conditions that would not have resulted in stasis were it not for the active compound treatment. Such activity of active compounds is determined by comparing the stasis-inducing effects (magnitude, kinetics) of injurious or disease stimuli alone with those in which the biological matter was pre-exposed, exposed concomitantly, exposed after, or any combination thereof, to the active compound. For example, as described in Example 11 of the instant patent application, pre-exposure of mice to 150 ppm H₂S in air caused an approximately two-fold drop in CO₂ production prior to exposure to hypoxia (5% O₂). Subsequently, CO₂ production in pre-treated mice fell ˜50-fold during hypoxia. In contrast, while CO₂ production in control, H₂S untreated mice also fell, the hypoxia survivability of the mice was not achieved, presumably since the mice died before stasis was achieved.

In the methods discussed above, an effective amount that is less than an amount that can induce stasis in an organism may be reduced with respect to duration and/or amount. That reduction may be a reduction in amount by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent, or any range derivable therein, of the amount to induce stasis. A reduction may be a reduction in duration (length of exposure time) by 1, 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7, days, 1, 2, 3, 4, 5 weeks, and/or 1, 2, 3, 4, 5, 6, 7, 8, 9, 10, 11, 12 months, or any range derivable therein. Alternatively, the reduction may be in terms of the overall effective amount provided to the biological matter, which may be a reduction of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82., 83, 84, 85, 86, 87, 88, 89, 90, 91, 92, 93, 94, 95, 96, 97, 98, 99 percent, or any range derivable therein, relative to the overall effective amount to induce stasis in an organism of that species and/or size.

Methods of the invention can involve employing an apparatus or system that maintains the environment in which biological matter is placed or exposed to. The invention includes an apparatus in which an active compound, particularly as a gas, is supplied. In some embodiments, the apparatus includes a container with a sample chamber for holding the biological matter, wherein the container is connected to a supply of gas comprising the oxygen antagonist(s). It is specifically contemplated that the container may be a solid container or it may flexible, such as a bag.

In some embodiments, the invention involves an apparatus for exposing biological matter to gas, the apparatus comprising: a container having a sample chamber with a volume of no greater than 775 liters; and a first gas supply in fluid communication with the sample chamber, the first gas supply including an active compound, such as carbon monoxide or hydrogen sulfide. In further embodiments, the apparatus also includes a cooling unit that regulates the temperature inside the sample chamber and/or a gas regulator that regulates the amount of an active compound in the chamber or the amount of an active compound in a solution that is in the chamber.

It is contemplated that there may be a gas supply for a second or additional gas or a second or additional gas supply for the active compound. The second gas supply may be connected with the sample chamber or it may be connected with the first gas supply. The additional gas, as discussed above, may be a non-toxic and/or non-reactive gas.

A gas regulator is part of the apparatus in some embodiments of the invention. One, two, three, or more gas regulators may be employed. In some cases, the gas regulator regulates the gas supplied to the sample chamber from the first gas supply. Alternatively, it regulates the gas supplied to the sample chamber or first gas supply from the second gas supply, or there may be a regulator for both the first and second gas supplies. It is further contemplated that any gas regulator can be programmed to control the amount of gas supplied to the sample chamber and/or to another gas supply. The regulation may or may not be for a specified period of time. There may be a gas regulator, which may or may not be programmable, for any gas supply directly or indirectly connected to the sample chamber. In some cases, the gas regulator is electronically programmable.

In some cases, the pressure and/or the temperature inside the chamber can be regulated with either a pressure regulator or temperature regulator, respectively. As with the gas regulator, these regulators may be electronically programmable. The apparatus of the invention may also have a cooling and/or heating unit to achieve the temperatures discussed above. The unit may or may not be electronically programmable.

In additional embodiments, the apparatus includes a wheeled cart on which the container rests or it may have one or more handles.

It is specifically contemplated that the invention includes an apparatus for whole organisms, in which the apparatus has: a container having a sample chamber; a first gas supply in fluid communication with the sample chamber, the first gas supply including the active compound(s); and an electronically-programmable gas regulator that regulates gas supplied to the sample chamber from the first gas supply.

In some embodiments, the apparatus also has a structure configured to provide a vacuum within the sample chamber.

Moreover, any active compound described in this application is contemplated for use with apparatuses of the invention. In specific embodiments, carbon monoxide can be administered using this apparatus. In other cases, a chalcogenide compound can be administered or a compound having the reducing agent structure. In still further embodiments, an active compound is administered using the apparatus. In specific embodiments, the invention covers a device or its use. In certain embodiments, the device is single dose delivery device. In other embodiments, the device is an inhaler or nebulizer. Moreover, it is contemplated that these devices may or may not be single dose delivery devices.

Additionally, the present invention concerns screening assays. In some embodiments, a candidate substance is screened for the ability to act as an oxygen antagonist or active compound, specifically including a protective metabolic agent. This can be done using any assay described herein, such as by measuring carbon dioxide output. Any substance identified as having exhibiting characteristics of an oxygen antagonist can be further characterized or tested. Moreover, it is contemplated that such a substance can be administered to biological matter to induce stasis or manufactured thereafter.

In certain embodiments, there are screening methods for active compounds, that can be used to treat or prevent shock. Furthermore, the methods of screening may be for any compounds that can effect the methods discussed herein. In some embodiments, there are screening methods involving a) exposing a mammal to a substance; b) placing the mammal under conditions that places them at risk for shock; c) comparing the outcome of mammals exposed to the substance to mammals not exposed to the substance, wherein an improvement in outcome in the mammals exposed to the substance, identifies the substance as a candidate active compound.

In some embodiments, the methods first involve identifying an appropriate substance to screen. In certain embodiments, the substance will be a chalcogenide, reducing agent, or have the structure of Formula I, Formula II, Formula III, or Formula IV, or any other compound discussed herein.

It is further contemplated that subsequent screens can be done in organisms considered higher or more complex than those used in preliminary or initial screens. Thus, it is contemplated that one or more cellular respiration factors will be assayed in these other organisms to further evaluate a candidate compound. In certain embodiments, subsequent screens involve the use of mice, rats, dogs, etc.

It is contemplated that a number of different organisms or biological matter (other cells or tissues) could be used and a number of different cellular respiration factors could be assayed in screening methods of the invention. In addition, it is contemplated that multiple such screens are performed at the same time in some embodiments of the invention.

It will of course be understood that in order for the substance to be considered a candidate active compound (or oxygen antagonist, or stasis inducer or protective metabolic agent, etc.) the substance must not kill the organism or cells in the assay and the effect must be reversible (that is, the characteristic that is altered needs to resume to its level before the exposure to the substance).

It is of course understood that any method of treatment can be used in the context of a preparation of a medicament for the treatment of or protection against the specified disease or condition. This includes, but is not limited to, the preparation of a medicament for the treatment of hemorrhagic or hematologic shock, wounds and tissue damage, hyperthermia, hypothermia, neurodegeneration, sepsis, cancer, and trauma. Moreover, the invention includes, but is not limited to, the preparation of a medicament for a treatment to prevent death, shock, trauma, organ or tissue rejection, damage from cancer therapy, neurodegeneration, and wound or tissue damage.

As discussed above, organismal stasis is not any of the following states: sleep, comatose, death, anesthetized, or grand mal seizure. However, it is contemplated in some embodiments of the invention, that such states are the desired goal of employing methods, compositions and articles of manufacture of the invention. Any embodiment discussed with respect to one aspect of the invention applies to other aspects of the invention as well. Moreover, embodiments may be combined.

Any embodiment involving “exposing” biological matter to an active compound may also be implemented so that biological matter is provided with the active compound or administered the active compound. The term “provide” is used according to its ordinary and plain meaning: “to supply or furnish for use” (Oxford English Dictionary), which, in the case of patients, may refer to the action performed by a doctor or other medical personnel who prescribes a particular active compound or administers it directly to the patient.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a block diagram illustrating a respiration gas delivery system according to embodiments of the present invention.

FIG. 2 is a schematic drawing illustrating a respiration gas delivery system according to embodiments of the present invention.

FIG. 3 is a schematic drawing illustrating a respiration gas delivery system according to further embodiments of the present invention.

FIG. 4 is a flowchart illustrating operations according to embodiments of the present invention.

FIG. 5 is a schematic drawing illustrating a tissue treatment gas delivery system according to embodiments of the present invention.

FIG. 6 is a flowchart illustrating operations according to embodiments of the present invention.

FIGS. 7A-B show negative pressure devices that can be used to deliver or administer active compounds.

FIG. 8 Short CO₂ pretreatment leads to greatest extension of anoxic survival. Adult flies were exposed to 100% CO₂ for the indicated time, the atmosphere was made anoxic by flushing with N₂, and then the tube was sealed. After 22 h, the tubes were opened to room air. The flies were allowed to recover for 24 h before scoring viability.

FIG. 9 CO₂ variably enhances anoxic survival. Adult flies were made anoxic in low-flow experiment, either directly from room air (no pretreatment) or after being exposed to 100% CO₂ for 10 min. After the indicated time, the tubes were opened to room air. The flies were allowed to recover for 24 h before scoring viability.

FIG. 10 50 ppm H₂S added to CO increases fraction of flies that survive anoxia. Adult flies were made anoxic in low-flow experiments, either directly from room air (no pretreatment) or after being exposed to 50 ppm H₂S balanced with CO.

FIG. 11 Survival of Mice in 5% Oxygen. Mice were exposed to either 30 minutes of room air before exposure to 5% O₂ (control; black line; n=9) or 10 minutes of room air followed by 20 minutes of 150 ppm H₂S before exposure to 5% O₂ (experimental; red line; n=20) and their length of survival measured. Experiments were stopped at 60 minutes and if the animals were still alive (all of the experimental, none of the controls) they were returned to their cage.

FIG. 12 H₂S Increases Survival at Lethal Oxygen Tensions. Chart showing results of experiment described in FIG. 11. The x-axis shows the time in minutes that the mice survived in the lower oxygen tensions. The dark bars show when H₂S is absent while the lighter bars show when H₂S is present. In the latter groups, mice were exposed to 150 ppm H₂S prior to the oxygen tension being reduced to between 5% and 2.5%. Survival times were measured and was at least 60 minutes in all the H₂S treated groups.

FIG. 13 H₂S Pre-treatment Enhances Survival of Mice Under Hypoxic Conditions. Mice were exposed to either 30 minutes of room air (No PT) or 10 minutes of room air followed by 20 minutes of 150 ppm H₂S (PT) before exposure to 5% O₂ (5%), 4% O₂ (4%), 5% O₂ for 1 hr followed by 4% O₂ (4%+1 hr 5%), or 5% O₂ for 1 hr followed by 3% O₂ (3%+1 hr 5%), and their length of survival measured. Experiments were stopped at 60 minutes and if the animals still alive were returned to their cage.

FIG. 14 Metabolic Rate of a Mouse in 5% Oxygen. A mouse was exposed to 10 minutes of room air followed by 20 minutes of 150 ppm H₂S prior to exposure to 5% O₂. Metabolic rate measured by CO₂ output. Pre-exposure CO₂ output was approximately 2500 ppm, after 20 minutes of H₂S then metabolic rate was down approximately 2-fold and after several hours of exposure to 5% O₂ the CO₂ output had dropped approximately 50-fold from pre-exposure levels to approximately 50 ppm. At hour 6 the mouse was returned to room air and allowed to recover. This data is from one of the mice included in FIG. 11 (experimental group).

FIG. 15 CO₂ Production During Transition to Lethal Hypoxia. Changes in CO₂ production upon transition to either 5% O₂ or 4% O₂ were measured in mice exposed to either room air for 30 minutes (No PT) or room air for 10 minutes followed by 150 ppm H₂S for 20 minutes (PT). In addition, the change in CO₂ production upon step-wise transition to 5% O₂ for 1 hr followed by 4% O₂ was measured. The percent change in CO₂ production is plotted with standard error indicated.

FIG. 16 shows results from a metabolic rate study using H₂S and 5% O₂ in mice. Relative carbon dioxide production and oxygen consumption of a mouse when pretreated and exposed to 5% O₂. Pre-Exposure levels were used to normalize the other observations. +6 hrs H₂S is the metabolic rate after a six hour exposure to 80 ppm H₂S; +6 hrs 5% O₂ is the metabolic rate after a 20 minute pretreatment with 150 ppm H₂S, which is followed by a six hour exposure to 5% O₂. These experiments were done at Tₐ=13° C. so they could be compared to our previous studies. Values are the means from three animals and the error bars represent one standard deviation. p-values indicate the significance between the bars with the same symbols.

FIG. 17 Adaptation to H₂S increases thermotolerance in wild-type C. elegans. A: Adapted animals survive at high temperature longer than unadapted controls. Adapted animals were exposed to high temperature in H₂S as young adults. The mean survival time of adapted animals was 65.5 h (n=136), compared to 9.1 h (n=96) for unadapted animals at high temperature in room air. B: Adaptation to H₂S is required to survive high temperature in H₂S Unadapted animals were moved to high temperature in environments that contain 50 ppm H₂S. The unadapted animals had a mean survival time of 2.1 h, whereas the group exposed to high temperature in room air survived for 7.3 h. C: Adapted animals require continuous presence of H₂S for increased survival at high temperature. Adapted animals at high temperature in room air survived 7.3 h, which is not significantly longer than unadapted controls (7.0 h).

FIG. 18 Adaptation to H₂S increases lifespan in C. elegans. A: Animals grown in H₂S have a longer lifespan than untreated controls. The mean lifespan of adapted animals in H₂S was 22.6±1.0 days (n=80), compared to 13.0±1.0 days for unadapted controls in room air (n=40). Maximum lifespan was also increased by 4 days. B: Exposure to H₂S in adulthood does not increase lifespan. Animals raised in house air were moved to H₂S-containing environments at the beginning of the lifespan experiment. The lifespan of these animals is 14.8±0.3 days, which is shorter than controls that remained in house air (mean lifespan 18.2±0.4 days).

FIG. 19 sir-2.1 is required for increased thermotolerance and lifespan upon adaptation to H₂S. A: Adaptation to H₂S does not increase thermotolerance of sir-2.1(ok434) animals. The mean survival time of sir-2.1(ok434) animals grown in H₂S and exposed to high temperature in H₂S is 9.8±0.3 h (n=20), which is not significantly longer than unadapted controls in room air (mean survival 9.6±0.3 h, n=20). B: Adaptation to H₂S does not increase the lifespan of sir-2.1(ok434) animals. The lifespan of sir-2.1(ok434) animals raised in H₂S is 20.0±1.6 days (n=47), statistically indistinguishable from control animals in room air (22.2±1.2 days, n=26).

### DESCRIPTION OF ILLUSTRATIVE EMBODIMENTS

### I. Stasis

In “stasis” or “suspended animation,” a cell, tissue or organ, or organism (collectively referred to as “biological material”) is living, but cellular functions necessary for cell division, developmental progression, and/or metabolic state are slowed or even stopped. This state is desirable in a number of contexts. Stasis can be used as a method of preservation by itself, or it may be induced as part of a cryopreservation regimen. Biological materials may be preserved for research use, for transportation, for transplantation, for therapeutic treatment (such as ex vivo therapy), and to prevent the onset of trauma, for example. Stasis with respect to entire organisms have similar uses. For instance, transportation of organisms could be facilitated if they had entered stasis. This might reduce physical and physiological damage to the organism by reducing or eliminating stress or physical injury. These embodiments are discussed in further detail below. Stasis may be beneficial by decreasing the need of the biological material for oxygen and, therefore, bloodflow. It may extend the period of time that biological material can be isolated from a life-sustaining environment and exposed to a death-inducing environment.

While recovery has been reported from accidental hypothermia for a relatively prolonged period of time (Gilbert et al., 2000), there has been recent interest in intentionally inducing suspended animation in organisms. (The discussion of any reference is not to be construed as an admission that the reference constitutes prior art. In fact, some references discussed herein would not be prior art with respect to the priority applications.) Controlled hyperthermia has been explored, as well as the administration of a cold flush of a solution into the aorta (Tisherman, 2004), induction of cardiac arrest (Behringer et al., 2003), or nitric oxide-induced suspended animation (Teodoro et al., 2004).

An organism in stasis is distinguishable from an organism under general anesthesia. For example, an organism in mild stasis (between about 2- and about 5-fold decrease in cellular respiration) that is exposed to room air will begin to shiver, while an organism under anesthesia will not. Also, an organism in mild stasis is anticipated to respond to a toe squeeze, while an organism under anesthesia usually does not. Consequently, stasis is not the same thing as being under anesthesia as it is commonly practiced.

CO₂ production is a direct marker of cellular respiration related to metabolism of an organism. This may be distinguished from “CO₂ evolution,” which refers to the amount of CO₂ blown out of the lungs. Certain active compounds, e.g., hydrogen sulfide, can inhibit carbonic anhydrase activity in the lungs, this inhibiting conversion of carbonate to CO₂ and its liberation from the pulmonary blood, thereby exhibiting an associated reduction in CO₂ evolution, without a corresponding decrease in cellular CO₂ production.

The present invention is based on the observation that certain compounds act as oxygen antagonists or protective metabolic agents, which may or may not induce reversible stasis or pre-stasis. Other patent applications discuss these compounds and uses include the following: U.S. patent application Ser. Nos. 10/971,576, 10/972,063, and 10/971,575; U.S. patent application Ser. No. 10/971,576; U.S. patent application Ser. No. 10/972,063; and U.S. patent application Ser. No. 10/971,575, all of which are hereby incorporated by reference.

A. Thermoregulation

Stasis in a warm-blooded animal will affect thermoregulation. Thermoregulation is a characteristic of so-called “warm-blooded” animals, which permits the organism to maintain a relatively constant core body temperature even when exposed to significantly altered (cold or hot) environmental temperatures. The ability to control thermoregulation by induction of stasis is one aspect of the invention, and permits uses similar to those discussed above.

Thermal regulation may be facilitated by placing of organisms, limbs or isolated organs or tissues into chambers/devices, the temperature of which can be controlled. For example, warm rooms or chamber-like devices similar to hyperbaric chambers may encompass an entire organism and be connected to thermo-regulatory apparti. Smaller devices such as blankets, sleeves, cuffs or gloves (e.g., CORE CONTROL cooling system by AVAcore Technologies, Palo Alto, Calif., U.S. Pat. No. 6,602,277) are also contemplated. Such chambers/devices may be used both to increase or reduce ambient temperatures.

B. Biological Matter

In addition to humans, the invention can be employed with respect to mammals of veterinary or agricultural importance including those from the following classes: canine, feline, equine, bovine, ovine, murine, porcine, caprine, rodent, lagomorph, lupine, and ursine. The invention also extends to fish and birds. Other examples are disclosed below.

The nonprovisional U.S. patent application Ser. Nos. 10/971,576, 10/972,063, and 10/971,575 disclose a variety of compounds and applications relevant to the present invention. All of these applications are hereby incorporated by reference in their entireties.

In certain aspects of the invention, the mammal is of the Order Monotremata, Marsupialia, Insectivora, Macroscelidia, Dermoptera, Chiroptera, Scandentia, Primates, Xenarthra, Pholidota, Tubulidentata, Lagomorpha, Rodentia, Cetacea, Carnivora, Proboscidea, Hyracoidea, Sirenia, Perissodactyla, or Artiodactyla.

3. Assays

Stasis can be measured by a number of ways, including by quantifying the amount of oxygen consumed by a biological sample, the amount of carbon dioxide produced by the sample (indirect measurement of cellular respiration), or characterizing motility.

To determine the rate of consumption of oxygen or the rate of production of carbon dioxide the biological matter is placed into a chamber that is sealed with two openings; for gas import and export. Gas (room air or other gases) is passed into the chamber at a given flow rate and out of the exit port to maintain approximately 1 atmosphere of pressure in the chamber. Before and after exposure to the chamber the gas is passed through a carbon dioxide detector and or an oxygen detector to measure (every second) the amount of each compound in the gas mixture. Comparison of these values over time gives the rate of oxygen consumption or carbon dioxide production.

### II. Active Compounds and Other Relevant Environmental Conditions

The present invention concerns methods, compositions and articles of manufacture involving one or more agents that can act on biological matter so as to produce a number of effects, including, but not limited to, inducing stasis, enhancing or increasing survivability, reversibly inhibiting metabolism, reducing cellular or organismal metabolism and activity, reducing the oxygen requirement, reducing or preventing damage, preventing ischemic damage, preventing aging or senescence, and/or a achieve a variety of therapeutic applications discussed herein. It certain embodiments, the agents are qualified as “active compounds.”

In other embodiments of the invention, there are methods comprising reversibly inhibiting metabolism of a cell and/or organism by providing an effective amount of an active compound. It is specifically contemplated that rotenone is not the compound employed in this method, or possibly other methods of the invention. Moreover, it is also contemplated that in some embodiments, rotenone is excluded as an active compound. Similarly, it is contemplated that nitric oxide may be excluded as an active compound.

In some embodiments, the agent is an oxygen antagonist, which may act directly or indirectly. Oxygen metabolism is a fundamental requirement for life in aerobic metazoans. Aerobic respiration accounts for the vast majority of energy production in most animals and also serves to maintain the redox potential necessary to carry out important cellular reactions. In hypoxia, decreased oxygen availability results in inefficient transfer of electrons to molecular oxygen in the final step of the electron transport chain. This inefficiency results in both a decrease in aerobic energy production and an increase in the production of damaging free radicals, mainly due to the premature release of electrons at complex III and the formation of O₂⁻ by cytochrome oxidase (Semenza, 1999). Limited energy supplies and free radical damage can interfere with essential cellular processes such as protein synthesis and maintenance of membrane polarities (Hochachka et al., 1996), and will ultimately lead to cell death.

In other embodiments, the agent is a protective metabolic agent. Metabolism is generally understood as referring to chemical processes (in a cell or organism) that are required for life; they involve a variety of reactions to sustain energy production and synthesize (anabolism) and break down (catabolism) complex molecules.

In certain embodiments of the invention, an active compound has a chemical structure as set forth as Formula I, II, III, or IV described herein, or is a precursor of Formula I or IV.

A variety of chemical structures and compounds are described herein. The following definitions apply to terms used to described these structures and compounds discussed herein:

“Alkyl,” where used, either alone or within other terms such as “arylalkyl”, “aminoalkyl”, “thioalkyl” “eyanoalkyl” and “hydroxyalkyl”, refers to linear or branched radicals having one to about twenty carbon atoms. The term “lower alkyl” refers to C₁-C₆ alkyl radicals. As used herein the term alkyl includes those radicals that are substituted with groups such as hydroxy, halo (such as F, Cl, Br, I), haloalkyl, alkoxy, haloalkoxy, alkylthio, cyano, isocyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidino, hydrazino, hydrazide, sodium sulfonyl (—SO₃Na), sodium sulfonylalkyl (—RSO₃Na), Examples of such radicals include, but are not limited to, methyl, ethyl, n-propyl, isopropyl, n-butyl, isobutyl, see-butyl, tert-butyl, pentyl, iso-amyl, hexyl and the like.

“Hydroxyalkyl” refers to an alkyl radical, as defined herein, substituted with one or more hydroxyl radicals. Examples of hydroxyalkyl radicals include, but are not limited to, hydroxymethyl, 2-hydroxyethyl, 2-hydroxypropyl, 3-hydroxypropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl, and 2-(hydroxymethyl)-3-hydroxypropyl, and the like.

“Arylalkyl” refers to the radical R′R— wherein an alkyl radical, “R” is substituted with an aryl radical “R′.” Examples of arylalkyl radicals include, but are not limited to, benzyl, phenylethyl, 3-phenylpropyl, and the like.

“Aminoalkyl” refers to the radical H₂NR′—, wherein an alkyl radical is substituted with am amino radical. Examples of such radicals include aminomethyl, amino ethyl, and the like. “Alkylaminoalkyl” refers to an alkyl radical substituted with an alkylamino radical.

“Alkylsulfonamido” refers to a sulfonamido group (—S(O)₂—NRR′) appended to an alkyl group, as defined herein.

“Thioalkyl” refers to wherein an alkyl radical is substituted with one or more thiol radicals. “Alkylthioalkyl” refers to wherein an alkyl radical is substituted with one or more alkylthio radicals. Examples include, but are not limited to, methylthiomethyl, ethylthioisopropyl, and the like. Arylthioalkyl” refers to wherein an alkyl radical, as herein defined, is substituted with one or more arylthio radicals.

“Carboxyalkyl” refers to the radicals —RCO₂H, wherein an alkyl radical is substituted with a carboxyl radical. Example include, but are not limited to, carboxymethyl, carboxyethyl, carboxypropyl, and the like.

“Alkylene” refers to bridging alkyl radicals.

The term “alkenyl” refers to an unsaturated, acyclic hydrocarbon radical in so much as it contains at least one double bond. Such alkenyl radicals contain from about 2 to about 20 carbon atoms. The term “lower alkenyl” refers to C₁-C₆ alkenyl radicals. As used herein, the term alkenyl radicals includes those radicals substituted as for alkyl radicals. Examples of suitable alkenyl radicals include propenyl, 2-chloropropenyl, buten-1-yl, isobutenyl, pent-1-en-1-yl, 2-2-methyl-1-buten-1-yl, 3-methyl-1-buten-1-yl, hex-2-en-1-yl, 3-hydroxyhex-1-en-1-yl, hept-1-en-1-yl, and oct-1-en-1-yl, and the like.

The term “alkynyl” refers to an unsaturated, acyclic hydrocarbon radical in so much as it contains one or more triple bonds, such radicals containing about 2 to about 20 carbon atoms. The term “lower alkynyl” refers to C₁-C₆ alkynyl radicals. As used herein, the term alkynyl radicals includes those radicals substituted as for alkyl radicals. Examples of suitable alkynyl radicals include ethynyl, propynyl, hydroxypropynyl, but-1-yn-1-yl, but-1-yn-2-yl, pent-1-yn-1-yl, pent-1-yn-2-yl, 4-methoxypent-1-yn-2-yl, 3-methylbut-1-yn-1-yl, hex-1-yn-1-yl, hex-1-yn-2-yl, hex-1-yn-3-yl, 3,3-dimethyl-1-butyn-1-yl radicals and the like.

“Alkoxy,” refers to the radical R′O—, wherein R′ is an alkyl radical as defined herein. Examples include, but are not limited to, methoxy, ethoxy, propoxy, butoxy, isopropoxy, tert-butoxy alkyls, and the like. “Alkoxyalkyl” refers to alkyl radicals substituted by one or more alkoxy radicals. Examples include, but are not limited to, methoxymethyl, ethoxyethyl, methoxyethyl, isopropoxyethyl, and the like.

“Alkoxycarbonyl” refers to the radical R—O—C(O)—, wherein R is an alkyl radical as defined herein. Examples of alkoxycarbonyl radicals include, but are not limited to, methoxycarbonyl, ethoxycarbonyl, sec-butoxycarbonyl, isopropoxycarbonyl, and the like. Alkoxythiocarbonyl refers to R—O—C(S)—.

“Aryl” refers to the monovalent aromatic carbocyclic radical consisting of one individual ring, or one or more fused rings in which at least one ring is aromatic in nature, which can optionally be substituted with one or more, preferably one or two, substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino, carbonyl, amidino, guanidino, hydrazino, hydrazide, sodium sulfonyl (—SO₃Na), sodium sulfonylalkyl (—RSO₃Na), unless otherwise indicated. Alternatively two adjacent atoms of the aryl ring may be substituted with a methylenedioxy or ethylenedioxy group. Examples of aryl radicals include, but are not limited to, phenyl, naphthyl, biphenyl, indanyl, anthraquinolyl, tert-butyl-phenyl, 1,3-benzodioxolyl, and the like.

“Arylsulfonamido” refers to a sulfonamido group, as defined herein, appended to an aryl group, as defined herein.

“Thioaryl” refers to an aryl group substituted with one or more thiol radicals.

“Alkylamino” refers to amino groups that are substituted with one or two alkyl radicals. Examples include monosubstituted N-alkylamino radicals and N,N-dialkylamino radicals. Examples include N-methylamino, N-ethylamino, N,N-dimethylamino, N,N-diethylamino, N-methyl, N-ethyl-amino, and the like.

“Aminocarbonyl” refers to the radical H₂NCO—. “Aminocarbonyalkyl” refers to the substitution of an alkyl radical, as herein defined, by one or more aminocarbonyl radicals.

“Amidyl” refers to RCO—NH—, wherein R is a H or alkyl, aryl, or heteroaryl, as defined herein.

“Imino carbonyl” refers to a carbon radical having two of the four covalent bond sites shared with an imino group. Examples of such imino carbonyl radicals include, for example, C═NH, C═NCH₃, C═NOH, and C═NOCH₃. The term “alkylimino carbonyl” refers to an imino radical substituted with an alkyl group, The term “amidino” refers to a substituted or unsubstituted amino group bonded to one of two available bonds of an iminocarbonyl radical. Examples of such amidino radicals include, for example, NH₂—C═NH, NH₂—C═NCH₃, NH—C═NOCH₃ and NH(CH₃)—C═NOH. The term “guanidino” refers to an amidino group bonded to an amino group as defined above where said amino group can be bonded to a third group. Examples of such guanidino radicals include, for example, NH₂—C(NH)—NH—, NH₂—C(NCH₃)—NH—, NH₂—C(NOCH₃)—NH—, and CH₃NH—C(NOH)—NH—. The term “hydrazino” refers to —NH—NRR′, where R and R′ are independently hydrogen, alkyl and the like. “Hydrazide” refers to —C(═O)—NH—NRR′.

The term “heterocyclyl” refers to saturated and partially saturated heteroatom-containing ring-shaped radicals having from 4 through 15 ring members, herein referred to as “C₄-C₁₅ heterocyclyl” selected from carbon, nitrogen, sulfur and oxygen, wherein at least one ring atom is a heteroatom. Heterocyclyl radicals may contain one, two or three rings wherein such rings may be attached in a pendant manner or may be fused. Examples of saturated heterocyclic radicals include saturated 3 to 6-membered heteromonocylic group containing 1 to 4 nitrogen atoms [e.g., pyrrolidinyl, imidazolidinyl, piperidino, piperazinyl, etc]; saturated 3 to 6-membered heteromonocyclic group containing 1 to 2 oxygen atoms and 1 to 3 nitrogen atoms [e.g. morpholinyl, etc.]; saturated 3 to 6-membered heteromonocyclic group containing 1 to 2 sulfur atoms and 1 to 3 nitrogen atoms [e.g., thiazolidinyl, etc.]. Examples of partially saturated heterocyclyl radicals include dihydrothiophene, dihydropyran, dihydrofuran and dihydrothiazole. Non-limiting examples of heterocyclic radicals include 2-pyrrolinyl, 3-pyrrolinyl, pyrrolindinyl, 1,3-dioxolanyl, 2H-pyranyl, 4H-pyranyl, piperidinyl, 1,4-dioxanyl, morpholinyl, 1,4-dithianyl, thiomorpholinyl, and the like. Such heterocyclyl groups may be optionally substituted with groups such as substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alkylamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino, carbonyl, amidino, guanidino, hydrazino, hydrazide, sodium sulfonyl (—SO₃Na), sodium sulfonylalkyl (—RSO₃Na).

“Heteroaryl” refers to monovalent aromatic cyclic radicals having one or more rings, preferably one to three rings, of four to eight atoms per ring, incorporating one or more heteroatoms, preferably one or two, within the ring (chosen from nitrogen, oxygen, or sulfur), which can optionally be substituted with one or more, preferably one or two substituents selected from substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alkylamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidino, hydrazino, hydrazide, sodium sulfonyl (—SO₃Na), sodium sulfonylalkyl (—RSO₃Na), unless otherwise indicated. Examples of heteroaryl radicals include, but are not limited to, imidazolyl, oxazolyl, thiazolyl, pyrazinyl, thienyl, furanyl, pyridinyl, quinolinyl, isoquinolinyl, benzofuryl, benzothiophenyl, benzothiopyranyl, benzimidazolyl, benzoxazolyl, benzothiazolyl, benzopyranyl, indazolyl, indolyl, isoindolyl, quinolinyl, isoquinolinyl, naphthyridinyl, benzenesulfonyl-thiophenyl, and the like.

“Heteroaryloxy” refers to heteroaryl radicals attached to an oxy radical. Examples of such radicals include, but are not limited to, 2-thiophenyloxy, 2-pyrimidyloxy, 2-pyridyloxy, 3-pyridyloxy, 4-pyridyloxy, and the like.

“Heteroaryloxyalkyl” refers to alkyl radicals substituted with one or more heteroaryloxy radicals. Examples of such radicals include 2-pyridyloxymethyl, 3-pyridyloxyethyl, 4-pyridyloxymethyl, and the like.

“Cycloalkyl” refers to monovalent saturated carbocyclic radicals consisting of one or more rings, typically one or two rings, of three to eight carbons per ring, which can typically be substituted with one or more substitutents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alkylamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino, carbonyl, amidino, guanidino, hydrazino, hydrazide, sodium sulfonyl (—SO₃Na), sodium sulfonylalkyl (—R SO₃Na), unless otherwise indicated, Examples of cycloalkyl radicals include, but are not limited to, cyclopropyl, cyclobutyl, 3-ethylcyclobutyl, cyclopentyl, cycloheptyl, and the like. “Cycloalkenyl” refers to radicals having three to ten carbon atoms and one or more carbon-carbon double bonds. Typical cycloalkenyl radicals have three to seven carbon atoms. Examples include cyclobutenyl, cyclopentenyl, cyclohexenyl, cycloheptenyl, and the like. “Cycloalkenylalkyl” refers to radicals wherein an alkyl radical, as defined herein, is substituted by one or more cycloalkenyl radicals.

“Cylcoalkoxy” refers to cycloalkyl radicals attached to an oxy radical. Examples include, but are not limited to, cyclohexoxy, cyclopentoxy and the like.

“Cylcoalkoxyalkyl” refers to alkyl radicals substituted one or more cycloalkoxy radicals. Examples include cyclohexoxyethyl, cyclopentoxymethyl, and the like.

Sulfinyl” refers to —S(O)—.

“Sulfonyl” refers to —S(O)₂—, wherein “alkylsulfonyl” refers to a sulfonyl radical substituted with an alkyl radical, RSO₂—, arylsulfonyl refers to aryl radicals attached to a sulfonyl radical. “Sulfonamido” refers to —S(O)₂—NRR′.

“Sulfonic acid” refers to —S(O)₂OH. “Sulfonic ester” refers to —S(O)₂OR, wherein R is a group such as an alkyl as in sulfonic alkyl ester.

“Thio” refers to —S—. “Alkylthio” refers to RS— wherein a thiol radical is substituted with an alkyl radical R. Examples include methylthio, ethylthio, butlythio, and the like. “Arylthio” refers to R′S—, wherein a thio radical is substituted with an aryl radical, as herein defined. “Examples include, but are not limited to, phenylthio, and the like. Examples include, but are not limited to, phenylthiomethyl and the like. F: “Alkylthiosulfonic acid” refers to the radical HO₃SR′S—, wherein an alkylthioradical is substituted with a sulfonic acid radical.

“Thiosulfenyl” refers to —S—SH.

“Acyl”, alone or in combination, refers to a carbonyl or thionocarbonyl group bonded to a radical selected from, for example, hydrido, alkyl, alkenyl, alkynyl, haloalkyl, alkoxy, alkoxyalkyl, haloalkoxy, aryl, heterocyclyl, heteroaryl, alkylsulfinylalkyl, alkylsulfonylalkyl, aralkyl, cycloalkyl, cycloalkylalkyl, cycloalkenyl, alkylthio, arylthio, amino, alkylamino, dialkylamino, aralkoxy, arylthio, and alkylthioalkyl. Examples of “acyl” are formyl, acetyl, benzoyl, trifluoroacetyl, phthaloyl, malonyl, nicotinyl, and the like.

The term “acylthiol” and “acyldisulfide” refers to the radicals RCOS— and RCOSS— respectively.

The term “thiocarbonyl” refers to the compounds and moieties which contain a carbon connected with a double bond to a sulfur atom —C(═S). “Alkylthiocarbonyl” refers to wherein a thiocarbonyl group is substituted with an alkyl radical, R. as defined herein, to form the monovalent radical R^{C}(═S)—. “Aminothiocarbonyl” refers to a thiocarbonyl group substituted with an amino group, NH₂C(═S)—.

“Carbonyloxy” refers to —OCOR.

“Alkoxycarbonyl” refers to —COOR.

“Carboxyl” refers to —COOH.

For those compounds with stereoisomers, all stereoisomers thereof, including cis/trans geometric isomers, diastereomers and the individual enantiomers are contemplated.

A. Carbon Monoxide

Carbon monoxide (CO) is a colorless, odorless, and tasteless gas that can be toxic to animals, including humans. According to the Center for Disease Control, more than 450 people unintentionally die from carbon monoxide each year.

It can be toxic to organisms whose blood carries oxygen to sustain its survival. It may be poisonous by entering the lungs through normal breathing and displacing oxygen from the bloodstream. Interruption of the normal supply of oxygen jeopardizes the functions of the heart, brain and other vital functions of the body. However, the use of carbon monoxide for medical applications is being explored (Ryter et al., 2004).

At amounts of 50 parts per million (ppm), carbon monoxide presents no symptoms to humans exposed to it. However, at 200 ppm, within two-three hours the carbon monoxide can cause a slight headache; at 400 ppm, within one to two hours it can cause a frontal headache that may become widespread within three hours; and, at 800 ppm it can cause dizziness, nausea, and/or convulsions within 45 minutes, and render the subject insensible within two hours. At levels of around 1000 ppm, an organism can expire after exposure for more than around 1-2 minutes.

Because of the well-known and well-documented toxic effects of carbon monoxide to an organism, it is thus surprising and unexpected that carbon monoxide can be used to induce stasis of and/or help preserve live biological samples. It is thus contemplated that carbon monoxide can be used for inducing stasis in isolated biological matter, such as blood-free biological matter (because of the effects that carbon monoxide has with respect to hemoglobin, which is a separate pathway than the one involved in inducing stasis).

In addition to exposure to carbon monoxide either to induce stasis or to limit or prevent any damage caused by a stasis-inducing agent, the invention contemplates that carbon monoxide may be used in combination with agents or methods that assist in the preservation and/or transplantation/grafting process of biological materials.

B. Chalcogenide Compounds

Compounds containing a chalcogen element; those in Group 6 of the periodic table, but excluding oxides, are commonly termed “chalcogenides” or “chalcogenide compounds (used interchangeably herein). These elements are sulfur (S), selenium (Se), tellurium (Te) and polonium (Po). Common chalcogenides contain one or more of S, Se and Te, in addition to other elements. Chalcogenides include elemental forms such as colloidal, micronized and/or nanomilled particles of S and Se. Chalcogenide compounds can be employed as reducing agents.

The present inventor, though not bound by the following theory, believes that the ability of chalcogenides to induce stasis in cells, and to permit modulation of core body temperature in animals, stems from the binding of these molecules to cytochrome oxidase. In so doing, chalcogenides inhibit or reduce the activity of oxidative phosphorylation. The ability of chalcogenides to block autonomous thermoregulation, i.e., to permit core body temperatures of “warm-blooded” animals to be manipulated through control of environmental temperatures, is believed to stem from the same mechanism as set forth above—binding to cytochrome oxidase, and blocking or reducing the activity of oxidative phosphorylation. Chalcogenides may be provided in liquid as well as gaseous forms.

Chalcogenides can be toxic, and at some levels lethal, to mammals. In accordance with the present invention, it is anticipated that the levels of chalcogenide should not exceed lethal levels in the appropriate environment. Lethal levels of chalcogenides may be found, for example in Material Safety Data Sheets for each chalcogenide or from information sheets available from the Occupational Safety and Health Administration (OSHA) of the US Government.

While carbon monoxide and chalcogenide compounds can both induce stasis by acting as an oxygen antagonist, they have different toxic effects that are separate from their abilities to induce stasis. Moreover, the concentrations needed to mediate a stasis effect are different because of the different affinities of cytochrome oxidase. While the affinity of cytochrome oxidase for oxygen is about 1:1 as compared to carbon monoxide, the affinity for H₂S appears on the order of about 300:1 as compared to oxygen. This impacts what toxic effects are observed with a stasis-inducing concentration. Thus, it is contemplated that chalcogenide compounds are particularly suited for inducing stasis of biological matter in whole organisms and of whole organisms.

It also may prove useful to provide additional stimuli to a biological matter before withdrawing the chalcogenide. In particular, it is envisioned that one may subject an animal to increased ambient temperature prior to removing the source of chalcogenide.

1. H₂S and Other Sulfur Containing Compounds

Hydrogen sulfide (H₂S) is a potentially toxic gas that is often associated with petrochemical and natural gas, sewage, paper pulp, leather tanning, and food processing. The primary effect, at the cellular level, appears to be inhibition of cytochrome oxidase and other oxidative enzymes, resulting in cellular hypoxia. Exposure to extreme levels (500 ppm) results in sudden collapse and unconsciousness, a so-called “knockdown” effect, followed by recovery. Post-exposure effects may persist for years, and include loss of coordination, memory loss, motor dysfunction, personality changes, hallucination and insomnia.

Most contact with H₂S, however, occurs well below such acute toxicity levels. Nonetheless, there is general concern over longterm contact at sub-acute levels. Some reports exist indicating persistent impairments in balance and memory, as well as altered sensory motor functions may occur in humans following chronic low-level H₂S exposure. Kilburn and Warshaw (1995); Kilburn (1999). Others have reported that perinatal exposure of rats to low (20 or 50 ppm) H₂S for 7 hours per day from gestation through post-natal day 21 resulted in longer dendritic branches with reduced aborization of cerebellar Purkinje cells. Other neurologic defects associated with relatively low levels of H₂S include altered brain neurotransmitter concentrations and altered neurologic responses, such as increased hippocampal theta EEG activity.

Behavioral toxicity was studied in rats exposed to moderate levels of H S. The results showed that H₂S inhibits discriminated avoidance responses immediately after the end of the exposure (Higuchi and Fukamachi, 1997), and also interferes with the ability of rats to learn a baited radial arm maze task (Partlo et al., 2001). In another perinatal study using 80 ppm H₂S, no neuropathological effects or altered motor activity, passive avoidance, or acoustic startle response in exposed rat pups was seen. Dorman et al. (2000). Finally, Struve et al. (2001) exposed rats to H₂S by gas at various levels for 3 hours per day on five consecutive days. Significant reductions in motor activity, water maze performance and body temperature following exposure to 80 ppm or greater H₂S were observed. Taken together, these reports indicate that H₂S can have a variety of effects on the biochemistry of mammalian tissues, but there is no clear pattern of response in terms of behavior.

Once dissolved in plasma, H₂S will be involved in a series of chemical reactions. The chemical reactions are: (1) the dissociation of the molecular H₂S to form the bisulfide ion, (2) the dissociation of the bisulfide ion to the sulfide ion, and (3) the self ionization of water. The reactions are given below:

H₂S_{(aq)} [symbol] HS_{(aq)}⁻+H_{(aq)}⁺

HS_{(aq)}⁻ [symbol] S_{(aq)}⁻²+H_{(aq)}⁺

H₂O [symbol] H_{(aq)}⁺+OH_{(aq)}⁻

Using the equilibrium constants K₁=1.039 E⁻⁰⁷, K₂=6.43 E⁻¹⁶ and K_{w}=1.019 E⁻¹⁴, at pH 7.4 the calculated amount of the different species relative to the total S concentration are approximately 23% H₂S and 77% HS⁻, while the amount of S²⁻ tends to zero.

The inventor uses an extractive alkylation technique coupled with gas chromatography and mass specific detection to quantify hydrogen sulfide (adapted from Hyspler et al., 2002). This method involves firstly adding a 50 μL sample of blood, serum or tissue extract that has been diluted in nitrogen purged deoxygenated water to a concentration of 1 mg/mL, together with 150 μL of a reaction buffer consisting of 5 mM benzalkonium chloride (BZK) in a saturated borate buffer. Added to this is first, 100 μL of a 15 μM solution of 4-chloro-benzyl methyl sulfide (4CBMS) in ethyl acetate and then 100 μL of a 20 mM solution of pentafluorobenzylbromide (PFB Br) in toluene. This solution is then sealed and incubated at 55° C. with rotation or shaking for 2 hr. After this incubation period, 200 μL of a saturated solution of KH₂PO₄ is then added, and the organic phase is removed and analyzed by gas chromatography and mass specific detection according to the methods described in Hyspler et al., 2002. These measurements are then compared to a standard curve generated using the same method described above, beginning with known standard concentrations ranging from 1 μM to 1 mM of Na₂S prepared in nitrogen purged deoxygenated H₂O, in order to determine the concentration of endogenous hydrogen sulfide levels. In order to analyze bound and/or oxidized sulfide levels, the same method is applied, except that a denaturing/reducing reaction buffer is used, which consists of 5 mM BZK with 1% tetraethylammonium hydroxide (TEAS) and 1 mM tris(2-carboxyethyl)-phosphine hydrochloride (TCEP) in saturated borate buffer, instead of the reaction buffer described above.

Typical levels of hydrogen sulfide contemplated for use in accordance with the present invention include values of about 1 to about 150 ppm, about 10 to about 140 ppm, about 20 to about 130 ppm, and about 40 to about 120 ppm, or the equivalent oral, intravenous or transdermal dosage thereof. Other relevant ranges include about 10 to about 80 ppm, about 20 to about 80 ppm, about 10 to about 70 ppm, about 20 to about 70 ppm, about 20 to about 60 ppm, and about 30 to about 60 ppm, or the equivalent oral, intravenous or transdermal thereof. It also is contemplated that, for a given animal in a given time period, the chalcogenide atmosphere should be reduced to avoid a potentially lethal build up of chalcogenide in the subject. For example, an initial environmental concentration of 80 ppm may be reduced after 30 min to 60 ppm, followed by further reductions at 1 hr (40 ppm) and 2 hrs (20 ppm).

In certain embodiments, colloidal sulfur may be employed. Colloidal sulfur can be prepared using a method loosely based on Monaghan and Garai, 1924. Colloidal sulfur may be provided to biological matter in any manner as described herein. The preparation involves the removal of thiol sulfur from thiosulfate using acid in the presences of serum proteins to form elemental sulfur molecules (S₆-S₂₀).

To one volume 2M sodium thiosulfate (Na₂S₂O₄), 2 volumes water and 1/10 volume serum are added. One volume 2N metaphosphoric acid is then added, and the mixture is allowed to react for up to 10 minutes. The pH of the mixture is then neutralized using sodium hydroxide (NaOH), followed by overnight dialysis against normal saline (0.9%). The liquid formulation with colloidal sulfur may then be administered to a subject. In certain embodiments, it is administered intravenously.

a. H₂S Precursors

The present invention also concerns the use of compounds and agents that can yield H₂S under certain conditions, such as upon exposure, or soon thereafter, to biological matter. It is contemplated that such precursors yield H₂S upon one or more enzymatic or chemical reactions.

3. Other Chalcogenides

In certain embodiments, the reducing agent structure compound is dimethylsulfoxide (DMSO), dimethylsulfide (OMS), methylmercaptan (CH₃SH), mercaptoethanol, thiocyanate, hydrogen cyanide, methanethiol (MeSH), or CS₂. In particular embodiments, the oxygen antagonist is CS₂, MeSH, or DMS. Compounds on the order of the size of these molecules are particularly contemplated (that is, within about 50% of their molecular weights).

Additional compounds that are envisioned as useful for inducing stasis include, but are not limited to, the following structures, many of which are readily available and known to those of skill in the art (identified by CAS number): 104376-79-6 (Ceftriaxone Sodium Salt); 105879-42-3; 1094-08-2 (Ethopropatine HCl); 1098-60-8 (Triflupromazine HCl); 111974-72-2; 113-59-7; 113-98-4 (Penicillin G K⁺); 115-55-9; 1179-69-7; 118292-40-3; 119478-56-7; 120138-50-3; 121123-17-9; 121249-14-7; 1229-35-2; 1240-15-9; 1257-78-9 (Prochlorperazine Edisylate Salt); 128345-62-0; 130-61-0 (Thioridazine HCl) 132-98-9 (Penicillin V K⁺); 13412-64-1 (Dicloxacillin Na⁺Hydrate); 134678-17-4; 144604-00-2; 146-54-3; 146-54-5 (Fluphenazine 211Cl); 151767-O₂-1; 159989-65-8; 16960-16-0 (Adrenocorticotropic Hormone Fragment 1-24); 1982-37-2; 21462-39-5 (Clindamycin HCl); 22189-31-7; 22202-75-1; 23288-49-5 (Probucol); 23325-78-2; 24356-60-3 (Cephapirin); 24729-96-2 (Clindamycin); 25507-04-4; 26605-69-6; 27164-46-1 (Cefazolin Na⁺); 2746-81-8; 29560-58-8; 2975-34-0; 32672-69-8 (Mesoridazine Benzene Sulfonate); 32887-01-7; 33286-22-5 ((⁺)-cis-Diltiazem HCl); 33564-30-6 (Cefoxitin Na⁺); 346-18-9; 3485-14-1; 3511-16-8; 37091-65-9 (Azlocillin Na⁺); 37661-08-8; 3819-00-9; 38821-53-3 (Cephradine); 41372-O₂-5; 42540-40-9 (Cefamandole Nafate); 4330-99-8 (Trimeprazine hemi-(⁺)-tartrate Salt); 440-17-5 Trifluoperazine 2HCl; 4697-14-7 (Ticarcillin 2Na⁺); 4800-94-6 (Carbenicillin 2Na⁺); 50-52-2; 50-53-3; 5002-47-1; 51481-61-9 (Cimetidine); 52239-63-1 (6-propyl-2-thiouracil); 53-60-1 (Promazine HCl); 5321-32-4; 54965-21-8 (Albendazole); 5591-45-7 (Thiothixene); 56238-63-2 (Cefuiroxime Na⁺); 56796-39-5 (Cefinetazole Na⁺); 5714-00-1; 58-33-3 (Promethazine HCl); 58-38-8; 58-39-9 (Perphenazine); 58-71-9 Cephalothin Na⁺); 59703-84-3 (Piperacillin Na⁺); 60-99-1 (Methotrimeprazine Maleate Salt); 60925-61-3; 61270-78-8; 6130-64-9 (Penicillin G Procaine Salt Hydrate); 61318-91-0 Sulconazole Nitrate Salt); 61336-70-7 Amoxicillin Trihydrate); 62893-20-3 Cefoperazone Na⁺); 64485-93-4 (Cefotaxime Na⁺); 64544-07-6; 64872-77-1; 64953-12-4 Moxalactam Na⁺); 66104-23-2 (Pergolide Mesylate Salt); 66309-69-1; 66357-59-3 (Ranitidine HCl); 66592-87-8 (Cefodroxil); 68401-82-1; 69-09-0 (Chlorpromazine HCl); 69-52-3 (Ampicillin Na⁺); 69-53-4 (Ampicillin); 69-57-8 Penicillin G Na⁺); 70059-30-2; 70356-03-5; 7081-40-5; 7081-44-9 (Cloxacillin Na⁺H₂O); 7177-50-6 Nafcillin Na+ 1120); 7179-49-9; 7240-38-2 (Oxacillin Na H₂O); 7246-14-2; 74356-00-6; 74431-23-5; 74849-93-7; 75738-58-8; 76824-35-6 (Famotidine); 76963-41-2; 79350-37-1; 81129-83-1; 84-02-6 (Prochlorperazine Dimaleate Salt); 87-08-1 (Phenoxymethylpenicillinic Acid); 87239-81-4; 91-33-8 (Benzthiazide); 91832-40-5; 94841-17-5; 99294-94-7; 154-42-7 (6-Thioguanine); 36735-22-5; 536-33-4 (Ethionamide); 52-67-5 (D-Penicillamine); 304-55-2 (Meso-2,3-Dimercaptosuccinic Acid); 59-52-9 2,3-Dimercapto⁺ propanol 6112-76-1 (6-mercaptopurine); 616-91-1 (N-acetyl-L-cysteine); 62571-86-2 (Captopril); 52-01-7 (spironolactone); and, 80474-14-2 (fluticasone propionate). Further compounds that are contemplated as possibly useful for stasis include those with the chemical structure of Formulas I or IV.

C. Other Antagonists or Active Compounds

1. Hypoxia and Anoxia

Hypoxia is a common natural stress and several well conserved responses exist that facilitate cellular adaptation to hypoxic environments. To compensate for the decrease in the capacity for aerobic energy production in hypoxia, the cell must either increase anaerobic energy production or decrease energy demand (Hochachka et al., 1996). Examples of both of these responses are common in metazoans and the particular response used depends, in general, on the amount of oxygen available to the cell.

In mild hypoxia, oxidative phosphorylation is still partially active, so some aerobic energy production is possible. The cellular response to this situation, which is mediated in part by the hypoxia-inducible transcription factor, HIF-1, is to supplement the reduced aerobic energy production by upregulating genes involved in anaerobic energy production, such as glycolytic enzymes and glucose transporters (Semenza, 2001; Guillemin et al., 1997). This response also promotes the upregulation of antioxidants such as catylase and superoxide dismutase, which guard against free radical-induced damage. As a result, the cell is able to maintain near normoxic levels of activity in mild hypoxia.

In an extreme form of hypoxia, referred to as “anoxia”—defined here as <0.001 kPa O₂—oxidative phosphorylation ceases and thus the capacity to generate energy is drastically reduced. In order to survive in this environment, the cell must decrease energy demand by reducing cellular activity (Hochachka et al., 2001). For example, in turtle hepatocytes deprived of oxygen, a directed effort by the cell to limit activities such as protein synthesis, ion channel activity, and anabolic pathways results in a 94% reduction in demand for ATP (Hochachka et al., 1996). In zebrafish (Danio rerio) embryos, exposure to anoxia leads to a complete arrest of the heartbeat, movement, cell cycle progression, and developmental progression (Padilla et al., 2001). Similarly, C. elegans respond to anoxia by entering into suspended animation, in which all observable movement, including cell division and developmental progression, ceases (Padilla et al., 2002; Van Voorhies et al., 2000). C. elegans can remain suspended for 24 hours or more and, upon return to normoxia, will recover with high viability. This response allows C. elegans to survive the hypoxic stress by reducing the rate of energetically expensive processes and preventing the occurrence of damaging, irrevocable events such as aneuploidy (Padilla et a., 2002; Nystul et al., 2003).

One recently discovered response is the hypoxia-induced generation of carbon monoxide by heme oxygenase-1 (Dulak et al., 2003). Endogenously produced carbon monoxide can activate signaling cascades that mitigate hypoxie damage through anti-apoptotic (Brouard et al., 2003) and anti-inflammatory (Otterbein et al., 2000) activity, and similar cytoprotective effects can be achieved in transplant models by perfusion with exogenous carbon monoxide (Otterbein et al., 2003; Amersi et al., 2002). At higher concentrations, carbon monoxide competes with oxygen for binding to iron-containing proteins, such as mitochondrial cytocbromes and hemoglobin (Gorman et al., 2003), though the cytoprotective effect that this activity may have in hypoxia has not been investigated.

Despite the existence of these sophisticated defense mechanisms against hypoxic damage, hypoxia is still often a damaging stress. For example, mammals have both heme oxygenase-1 and HIF-1, and some evidence suggests that suspended animation is possible in mammals as well (Bellamy et al., 1996; Alam et al., 2002). Yet, hypoxic damage due to trauma such as heart attack, stroke or blood loss is a major cause of death. The understanding of the limitations of the two fundamental strategies for surviving hypoxic stress, remaining animated or suspending animation, is hampered by the fact that it has been based on studies in a variety of systems under a variety of conditions.

“Hypoxia” occurs when the normal physiologic levels of oxygen are not supplied to a cell or tissue. “Normoxia” refers to normal physiologic levels of oxygen for the particular cell type, cell state or tissue in question. “Anoxia” is the absence of oxygen. “Hypoxic conditions” are those leading to cellular hypoxia. These conditions depend on cell type, and on the specific architecture or position of a cell within a tissue or organ, as well as the metabolic status of the cell. For purposes of the present invention, hypoxic conditions include conditions in which oxygen concentration is at or less than normal atmospheric conditions, that is less that 20.8, 20, 19, 18, 17, 16, 15, 14, 13, 12, 11, 10, 9, 8, 7, 6, 5, 4, 3, 2, 1, 0.5, 0%; alternatively, these numbers could represent the percent of atmosphere at 1 atmosphere of pressure (101.3 kPa). An oxygen concentration of zero percent defines anoxic conditions. Thus, hypoxic conditions include anoxic conditions, although in some embodiments, hypoxic conditions of not less than 0.5% are implemented. As used herein, “normoxic conditions” constitute oxygen concentrations of around 20.8% or higher.

Standard methods of achieving hypoxia or anoxia are well established and include using environmental chambers that rely on chemical catalysts to remove oxygen from the chamber. Such chambers are available commercially from, for example, BD Diagnostic Systems (Sparks, Md.) as GASPAK Disposable Hydrogen+Carbon Dioxide Envelopes or BIO-BAG Environmental Chambers. Alternatively, oxygen may be depleted by exchanging the air in a chamber with a non-oxygen gas, such as nitrogen. Oxygen concentration may be determined, for example using a FYRITE Oxygen Analyzer (Bacharach, Pittsburgh Pa.).

It is contemplated that methods of the invention can use a combination of exposure to an active compound and alteration of oxygen concentrations compared to room air. Moreover, the oxygen concentration of the environment containing biological matter can be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range derivable therein. Moreover, it is contemplated that a change in concentration can be any of the above percentages or ranges, in terms of a decrease or increase compared to room air or to a controlled environment.

2. Mitochondrial Targeting Agents

Selectively targeting mitochondria is considered an embodiment of the invention in some aspects so as to enhance activity. Such selective mitochondrial targeting has been accomplished by conjugating agents to a lipophilic triphenylphosphonium cation, which readily cross lipid bilayers and accumulate approximately a 1000 fold within the mitochondrial matrix drive by the large potential (150 to −180 mv) across the mitochondrial inner membrane. Analogs of both vitamin E and ubiquinone have been prepared and used to successfully target mitochondria. (Smith et al., 1999; Kelso et al., 2001; Dhanasekaran et al. 2004). A thiol, thiobutyltriphosphonium bromide, has been prepared and used to target mitochondria wherein it accumulated several hundred-fold (Burns et al., 1995; Burns & Murphy, 1997).

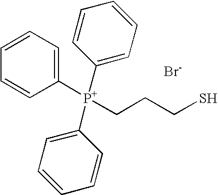

Such conjugates would appear to be suitable candidates for active compounds. In addition to free thiol agents, thiosulfenyl substituted compounds, (H—S—S—R) may be useful. It is contemplated that in some embodiments the agents have the structure:

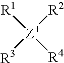

where Z is P or N;
R¹, R² and R³ are aryl, heteroaryl, alkylaryl, cycloalkyl, or alkyl (suitably phenyl, benzyl, tolyl, pyridyl, cyclohexyl, C₃-C₁₀ alkyl, optionally halogenated);
R⁴ is —R⁵SR⁶, wherein R⁵ is C₁-C₁₀ alkyl, R⁶ is H or SH, SO₃H, or PO₃H.

### III. Therapeutic or Preventative Applications

A. Trauma

In certain embodiments, the present invention may find use in the treatment of patients who are undergoing, or who are susceptible to trauma. Trauma may be caused by external insults, such as burns, wounds, amputations, gunshot wounds, or surgical trauma, internal insults, such as stroke or heart attack that result in the acute reduction in circulation, or reductions in circulation due to non-invasive stress, such as exposure to cold or radiation. On a cellular level, trauma often results in exposure of cells, tissues and/or organs to hypoxia, thereby resulting in induction of programmed cell death, or “apoptosis.” Systemically, trauma leads to the induction of a series of biochemical processes, such as clotting, inflammation, hypotension, and may give rise to shock, which if it persists may lead to organ dysfunction, irreversible cell damage and death. Biological processes are designed to defend the body against traumatic insult; however they may lead to a sequence of events that proves harmful and, in some instances, fatal.

Therefore, the present invention contemplates protection of tissues, organs, limbs and even whole organisms from the detrimental effects of trauma. In a specific scenario, where medical attention is not readily available, induction of stasis in vivo or ex vivo, alternatively in conjunction with reduction in the temperature of the tissue, organ or organism, can “buy time” for the subject, either by bringing medical attention to the subject, or by transporting the subject to the medical attention. The present invention also contemplates methods for inducing tissue regeneration and wound healing by prevention/delay of biological processes that may result in delayed wound healing and tissue regeneration. In this context, in scenarios in which there is a substantial wound to the limb or organism, the induction of stasis induction of stasis in vivo or ex vivo, alternatively in conjunction with reduction in the temperature of the tissue, organ or organism, can aid in the wound healing and tissue regeneration process by managing the biological processes that inhibit healing and regeneration.

In addition to wound healing and hemorrhagic shock discussed below, methods of the invention can be implemented to prevent or treat trauma such as cardiac arrest or stroke. The invention has particular importance with respect to the risk of trauma from emergency surgical procedures, such as thoractomy, laparotomy, and splenic transection.

1. Wound Healing

In many instances, wounds and tissue damage are intractable or take excessive periods of time to heal. Examples are chronic open wounds (diabetic foot ulcers and stage 3 & 4 pressure ulcers), acute and traumatic wounds, flaps and grafts, and subacute wounds (i.e., dehisced incisions). This may also apply to other tissue damage, for example burns and lung damage from smoke/hot air inhalation.

Previous experiments show hibernation to be protective against injury (e.g., pin's in brains), therefore it may have healing effects. Consequently, this technology may be useful in the control of wound healing processes, by bringing the tissue into a more metabolically controlled environment. More particularly, the length of time that cells or tissue are kept in stasis can vary depending on the injury. In some embodiments of the invention, biological matter is exposed to an active compound for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months or more.

2. Hematologic Shock (Hemorrhagic Shock)

Accordingly, the invention concerns inducing a whole body state (as exhibited by apnea and/or lack of skeletal muscle movement) using H₂S (or other oxygen antagonist or other active compound), to preserve the patient's vital organs and life. This will allow for transport to a controlled environment (e.g., surgery), where the initial cause of the shock can be addressed, and then the patient brought back to normal function in a controlled manner. For this indication, the first hour after injury, referred to as the “golden hour,” is crucial to a successful outcome. Stabilizing the patient in this time period is the major goal, and transport to a critical care facility (e.g., emergency room, surgery, etc.) where the injury can be properly addressed. Thus, it would be ideal to maintain the patient in stasis to allow for this and to address immediate concerns such as source of shock, replenish blood loss, and reestablish homeostasis. While this will vary significantly, in most cases, the amount of time stasis will be maintained is between about 6 and about 72 hours after injury. In some embodiments of the invention, biological matter is exposed to an active compound for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days or more, and any range or combination therein.

The biology of lethal hemorrhage and the physiological events that lead to shock and ultimately death are not fully understood. However, there are mechanisms through which H₂S could reduce the lethal effects of ischemic hypoxia. Hydrogen sulfide inhibits cytochrome C oxidase and could reduce oxygen demand by inhibiting this enzyme³. Decreased oxygen demand may reduce the deleterious effects of low oxygen levels including a reduction of metabolic acidosis. Furthermore, tissue sulfydryl levels decrease during shock (Beck et al., 1954). Exogenous H₂S may prevent this hyposulfidic state and maintain sulfur homeostasis.

Hydrogen sulfide is naturally produced in animals and exhibits potent biological activities (Kamoun, 2004). Most proteins contain disulfide linked cysteine residues, and the reversible conversion from free thiol to disulfide can regulate specific enzyme activities (Ziegler, 1985). Furthermore, sulfide is electronegative and exhibits high affinity for transition metals. Proteins containing transition metal atoms, such as cytochrome oxidase, can be profoundly affected by H₂S. And finally, metabolism of H₂S into other molecules containing reduced sulfur increases the number of thiols that may exhibit specific biological activity. In addition to (or perhaps because of) these potential modes of action, H₂S may exert effects on cardiopulmonary, neuroendocrine, immune, and/or hemostatic systems that ultimately prove beneficial in injury and disease.

B. Other Therapeutic Applications

In certain embodiments, active compounds of the present invention can enhance survivability and/or increase thermotolerance in a subject. For example, exposing a subject to H₂S may impart increased thermotolerance and survivability (e.g., longevity or lifespan) to that subject.

Sirtuins have become a research topic of interest recently due in part to the discovery that one member of this family, Sir2, acts as a yeast longevity factor (Kaeberlein et al., 1999) and a similar gene, sir 2.1, has been detected in C. elegans (Tissenbaum and Guarente, 2001; Guarente, 2005). As putative metabolic sensors in cells and organisms, sirtuins have been linked to age-related diseases such as cancer, diabetes and neurodegenerative disorders (Long and Kennedy, 2006). Sirtuins are ubiquitously found across nearly all organisms studied thus far, and also play a key role in an organism's response to stressors such as heat or starvation. For example, starving of yeast cells can lead to extended lifespans, resulting in increases in Sir2 activity; removal of the sir2 gene eliminates the life-extending effect of calorie restriction. Guarente, 2005. Examples of sirtuin proteins in mammals include SIRT1 (the analog of yeast Sir2), SIRT2, SIRT3, SIRT4, SIRT5, SIRT6 and SIRT7. Sirtuins and the modulation thereof have also been associated with ischemia/reperfusion and chemoprotection.

Amino acid sequences of several mammalian sirtuins are publicly available. See, e.g., GenBank Accession Nos. Q96EB6, AAH12499, NP_{—}036370 and AAD40849 for human SIRT1; GenBank Accession Nos. Q923E4 and NP_{—}062786 for mouse SIRT1; GenBank Accession Nos. NP_{—}085096, NP_{—}036369, AAH03547 and AAH03012 for human SIRT2; GenBank Accession Nos. AAH86545 and NP_{—}001008369 for rat SIRT2; GenBank Accession No. NP_{—}071877 for mouse SIRT2; GenBank Accession Nos. NP_{—}07878 and AAH25878 for mouse SIRT3; GenBank Accession Nos. NP_{—}036371, AAH01042 and AAD40851 for human SIRT3.

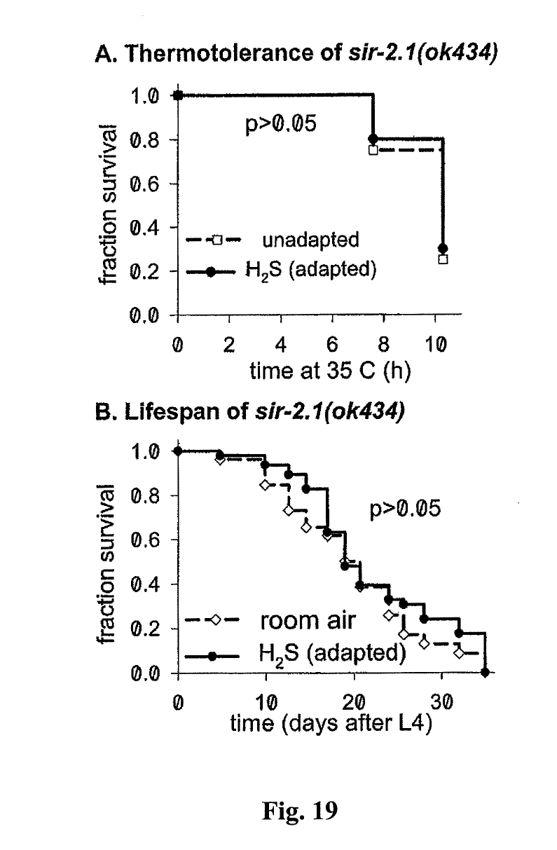

Accordingly, in particular aspects of the invention, an active compound may modulate the activity of one or more sirtuin proteins of a subject. Sirtuin modulation by an active compound may be involved, for example, in the inducement, regulation, pre-treatment or treatment of any of the conditions described herein, such as cardioplegia, myocardial infarction, ischemia/reperfusion injury, sepsis, antiinflammatory-related events, stasis, anoxia, hypoxia, or hemorrhagic shock. Such modulation may, for example, result in increased thermotolerance and/or enhanced survivability in biological matter. Sirtuin modulation by an active compound may influence ischemia/reperfusion and/or enhance chemoprotection. For example, the inventors have discovered that surprisingly, SIR-2 protein in C. elegans is required for increased thermotolerance and lifespan resulting from exposure to H₂S. See FIG. 19.

In some embodiments, detection of sirtuin gene expression may be the basis for a diagnostic or prognostic test for determining the effectiveness of an active compound for treatment purposes. In certain embodiments, such a diagnostic or prognostic test could determine the effectiveness of pretreating a subject with an active compound.

### IV. Screening Applications

In still further embodiments, the present invention provides methods for identifying compounds that can be used to enhance survivability in patients at risk for shock. These assays may comprise random screening of large libraries of candidate substances; alternatively, the assays may be used to focus on particular classes of compounds selected with an eye towards attributes that are believed to make them more likely to act as active compounds. For example, a method generally comprises:
(a) providing a test compound to a subject;
(b) exposing the subject to conditions that place the subject at risk for shock
(c) evaluating any increase in survivability in the subject provided with the candidate compound, wherein an increase indicates the test compound is a candidate therapeutic agent.

Alternatively, in other methods of the invention there are methods comprising:
(a) providing a test compound to a subject;
(b) evaluating the subject for lack of skeletal muscle movement and/or apnea, wherein the presence of either indicates the test compound is a candidate therapeutic agent.

It will, of course, be understood that all the screening methods of the present invention are useful in themselves notwithstanding the fact that effective candidates may not be found. The invention provides methods for screening for such candidates, not solely methods of finding them. However, it will also be understand that a test compound may be identified as a candidate compound according to one or more assays, meaning that the compound appears to have some ability to lead to a physiological effect, such as by inducing apnea or reducing the risk of dying from hemorrhagic shock. Screening, in some embodiments, involves using an assay described in the Examples or elsewhere in the disclosure to identify a candidate compound. Moreover, in addition to or instead of the method described in this section, a candidate compound may be tested for activity either as an oxygen antagonist or as another compound having a property of an active compound, such as protective metabolic agent or therapeutic substance. Some embodiments of screening methods are provided above.

A candidate compound may be further characterized or assayed. Moreover, the candidate compound may be used in a subsequent in vivo animal or animal model (as discussed below) or be used in further in vivo animals or animal models, which may involve the same species of animals or in different animal species.

Furthermore, it is contemplated that candidate identified according to embodiments of the invention may also be manufactured after screening. Also, biological matter may be exposed to or contacted with a candidate compound according to methods of the invention, particularly with respect to therapeutic or preservation embodiments.

A. Modulators

As used herein the term “candidate substance” refers to any molecule that may induce apnea in a subject, for example. One may also acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

In addition to the modulating compounds initially identified, the inventor also contemplates that other structurally similar compounds may be formulated to mimic the key portions of the structure of the modulators. Such compounds, which may include peptidomimetics of peptide modulators, may be used in the same manner as the initial modulators.

B. In Vivo Assays

In vivo assays involve the use of various animal models. Due to their size, ease of handling, and information on their physiology and genetic make-up, mice are a preferred embodiment. However, other animals are suitable as well, including rats, rabbits, hamsters, guinea pigs, gerbils, woodchucks, mice, cats, dogs, sheep, goats, pigs, cows, horses and monkeys (including chimps, gibbons and baboons). Fish are also contemplated for use with in vivo assays, as are nematodes. Assays for modulators may be conducted using an animal model derived from any of these species.

In such assays, one or more candidate substances are administered to an animal, and the ability of the candidate substance(s) to induce apnea, survive significant blood loss, or endow on the biological material the ability to survive going into shock, as compared to an inert vehicle (negative control) and H₂S (positive control), identifies a modulator. Treatment of animals with test compounds will involve the administration of the compound, in an appropriate form, to the animal. Administration of the candidate compound (gas or liquid) will be by any route that could be utilized for clinical or non-clinical purposes, including but not limited to oral, nasal (inhalation or aerosol), buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

### V. Modes of Administration and Pharmaceutical Compositions

An effective amount of a pharmaceutical composition of a chalcogenide, oxygen antagonist, or other active compound, generally, is defined as that amount sufficient to detectably ameliorate, reduce, minimize or limit the extent of the condition of interest. More rigorous definitions may apply, including elimination, eradication or cure of disease.

A. Administration

The routes of administration of a chalcogenide will vary, naturally, with the location and nature of the condition to be treated, and include, e.g., inhalation, intradermal, transdermal, parenteral, intravenous, intramuscular, intranasal, subcutaneous, percutaneous, intratracheal, intraperitoneal, intratumoral, perfusion, lavage, direct injection, and oral administration and formulation. As detailed below, active compounds may be administered as medical gases by inhalation or intubation, as injectable liquids by intravascular, intravenous, intra-arterial, intracerebroventicular, intraperitoneal, subcutaneous administration, as topical liquids or gels, or in solid oral dosage forms.

Moreover, the amounts may vary depending on the type of biological matter (cell type, tissue type, organism genus and species, etc.) and/or its size (weight, surface area, etc.). It will generally be the case that the larger the organism, the larger the dose. Therefore, an effective amount for a mouse will generally be lower than an effective amount for a rat, which will generally be lower than an effective amount for a dog, which will generally be lower than an effective amount for a human. The effective concentration of hydrogen sulfide to achieve stasis in a human depends on the dosage form and route of administration. For inhalation, in some embodiments effective concentrations are in the range of 50 ppm to 500 ppm, delivered continuously. For intravenous administration, in some embodiments effective concentrations are in the range of 0.5 to 50 milligrams per kilogram of body weight delivered continuously.

Similarly, the length of time of administration may vary depending on the type of biological matter (cell type, tissue type, organism genus and species, etc.) and/or its size (weight, surface area, etc.) and will depend in part upon dosage form and route of administration. In particular embodiments, an active compound is provided for about or at least 30 seconds, 1 minute, 2 minutes, 3 minutes, 5 minutes, 10 minutes, 15 minutes, 30 minutes, 1 hour, 2 hours, 3 hours, four hours five hours, six hours, eight hours, twelve hours, twenty-four hours, or greater than twenty-four hours. An active compound may be administered in a single dos or multiple doses, with varying amounts of time between administered doses.

In the case of transplant, the present invention may be used pre- and or post-operatively to render host or graft materials quiescent. In a specific embodiment, a surgical site may be injected or perfused with a formulation comprising a chakcogenide. The perfusion may be continued post-surgery, for example, by leaving a catheter implanted at the site of the surgery.

B. Injectable Compositions and Formulations

The preferred methods for the delivery of an active compound of the present invention are inhalation, intravenous injection, perfusion of a particular area, and oral administration. However, the pharmaceutical compositions disclosed herein may alternatively be administered parenterally, intradermally, intramuscularly, transdermally or even intraperitoneally as described in U.S. Pat. Nos. 5,543,158, 5,641,515 and 5,399,363 (each specifically incorporated herein by reference in its entirety).

Solutions of the active compounds may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

C. Intravenous Formulations

In one embodiment, active compounds of the invention may be formulated for parenteral administration (e.g., intravenous, intra-arterial). In the cases where the active compound is a gas at room temperature, a solution containing a known and desired concentration of the gas molecule dissolved in a liquid or a solution for parenteral administration is contemplated. Preparation of the active compound solution may be achieved by, for example, contacting (e.g., bubbling or infusing) the gas with the solution to cause the gas molecules to dissolve in the solution. Those skilled in the art will recognize that the amount of gas that dissolves in the solution will depend on a number of variables including, but not limited to, the solubility of the gas in the liquid or solution, the chemical composition of the liquid or solution, its temperature, its pH, its ionic strength, as well as the concentration of the gas and the extent of contacting (e.g., rate of and duration of bubbling or infusing). The concentration of the active compound in the liquid or solution for parenteral administration can be determined using methods known to those skilled in the art. The stability of the active compound in the liquid or solution can be determined by measuring the concentration of the dissolved oxygen antagonist after varying intervals of time following preparation or manufacture of the oxygen antagonist solution, where a decrease in the concentration of the oxygen antagonist compared to the starting concentration is indicative of loss or chemical conversion of the active compound.

In some embodiments, there is a solution containing a chalcogenide compound is produced by dissolving a salt form of the chalcogenide into sterile water or saline (0.9% sodium chloride) to yield a pharmaceutically acceptable intravenous dosage form. The intravenous liquid dosage form may be buffered to a certain pH to enhance the solubility of the chalcogenide compound or to influence the ionization state of the chalcogenide compound. In the cases of hydrogen sulfide or hydrogen selenide, any of a number of salt forms known to those skilled in the art may suffice, including, but not limited to, sodium, calcium, barium, lithium, or potassium. In another preferred embodiment, sodium sulfide or sodium selenide is dissolved in sterile phosphate buffered saline and the pH is adjusted to 7.0 with hydrochloric acid to yield a solution of known concentration which can be administered to a subject intravenously or intrarterially.

It is contemplated that in some embodiments, a pharmaceutical composition of the invention is a saturated solution with respect to the active compound. The solution can be any pharmaceutically acceptable formulation, many of which are well known, such as Ringer's solution. In certain embodiments, the concentration of the active compound is about, at least about, or at most about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0 M or more, or any range derivable therein (at standard temperature and pressure (STP)). With H₂S, for example, in some embodiments, the concentration can be from about 0.01 to about 0.5 M (at STP). It is specifically contemplated the above concentrations may be applied with respect to carbon monoxide and carbon dioxide in a solution separately or together.

Furthermore, when administration is intravenous, it is contemplated that the following parameters may be applied. A flow rate of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 gtts/min or μgtts/min, or any range derivable therein. In some embodiments, the amount of the solution is specified by volume, depending on the concentration of the solution. An amount of time may be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, and/or 1, 2, 3, 4, 5, 6, 7, 8, 9, 10, 11, 12 months, or any range derivable therein.

Volumes of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mls or liters, or any range therein, may be administered overall or in a single session.

In some embodiments, the solution of the active compound for parenteral administration is prepared in a liquid or solution in which the oxygen has been removed prior to contacting the liquid or solution with the active compound. Certain oxygen antagonists, in particular certain chalcogenide compounds (e.g., hydrogen sulfide, hydrogen selenide), are not stable in the presence of oxygen due to their ability to react chemically with oxygen, leading to their oxidation and chemical transformation. Oxygen can be removed from liquids or solutions using methods known in the art, including, but not limited to, application of negative pressure (vacuum degasing) to the liquid or solution, or contacting the solution or liquid with a reagent which causes oxygen to be bound or “chelated”, effectively removing it from solution.

In another embodiment, the solution of the oxygen antagonist for parenteral administration may be stored in a gas-tight container. This is particularly desirable when the oxygen has previously been removed from the solution to limit or prevent oxidation of the oxygen antagonist. Additionally, storage in a gas-tight container will inhibit the volatilization of the oxygen antagonist gas from the liquid or solution, allowing a constant concentration of the dissolved oxygen antagonist to be maintained. Gas-tight containers are known to those skilled in the art and include, but are not limited to, “i.v. bags” comprising a gas impermeable construction material, or a sealed glass vial. To prevent exposure to air in the gas-tight storage container, an inert gas, such as nitrogen or argon, may be introduced into the container prior to closure.

D. Topical Formulations and Uses Thereof

Methods and compositions of the present invention are useful for inducing stasis in superficial layers of the skin and oral mucosa, including, but not limited to, hair follicle cells, capillary endothelial cells, and epithelial cells of the mouth and tongue. Radiation therapy and chemotherapy for the treatment of cancer damage normal cells in the hair follicles and oral mucosa, leading to the unintended, but debilitating side effects of cancer therapy, hair loss and oral mucositis, respectively. Induction of stasis in hair follicle cells and/or the vascular cells that supply blood to the hair follicles may slow, limit or prevent damage to hair follicle cells and the resultant hair loss that accompanies radiation therapy and chemotherapy, or other alopecia, male-pattern baldness, female-pattern baldness, or other absence of the hair from skin areas where it normally is present. Induction of stasis in oral epithelial and mesenchymal cells may slow, limit or prevent damage to cells lining the mouth, esophagus and tongue and the resultant painful condition of oral mucositis.

In certain embodiments the active compound is administered topically. This is achieved by formulating the active compound in a cream, gel, paste, or mouthwash and applying such formulation directly to the areas that require exposure to the active compound (e.g., scalp, mouth, tongue, throat).

The topical compositions of this invention can be formulated as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight.

Lotions may be conveniently prepared by dissolving the active ingredient, in a suitable high molecular weight alcohol such as propylene glycol or polyethylene glycol.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

E. Solid Dosage Forms

Pharmaceutical compositions include solid dosage forms in which the active compound is trapped, or sequestered, in a porous carrier framework that is capable of achieving a crystalline, solid state. Such solid dosage forms with the capacity for gas storage are known in the art and can be produced in pharmaceutically acceptable forms (e.g., Yaghi et al. 2003). A particular advantage of such a pharmaceutical composition pertains to chalcogenide compounds (e.g., hydrogen sulfide, carbon monoxide, hydrogen selenide), which can be toxic to certain mammals at certain concentrations in their free form. In certain embodiments, the compound may be formulated for oral administration.

F. Perfusion Systems

A perfusion system for cells may be used to expose a tissue or organ to an active compound in the form of a liquid or a semi-solid. Perfusion refers to continuous flow of a solution through or over a population of cells. It implies the retention of the cells within the culture unit as opposed to continuous-flow culture, which washes the cells out with the withdrawn media (e.g., chemostat). Perfusion allows for better control of the culture environment (pH, pO₂, nutrient levels, active compound levels, etc.) and is a means of significantly increasing the utilization of the surface area within a culture for cell attachment.

The technique of perfusion was developed to mimic the cells milieu in vivo where cells are continuously supplied with blood, lymph, or other body fluids. Without perfusion of a physiological nutrient solution, cells in culture go through alternating phases of being fed and starved, thus limiting full expression of their growth and metabolic potential. In the context of the present invention, a perfusion system may also be used to perfuse cells with an oxygen antagonist to induce stasis.

Those of skill in the art are familiar with perfusion systems, and there are a number a perfusion systems available commercially. Any of these perfusion systems may be employed in the present invention. One example of a perfusion system is a perfused packed-bed reactor using a bed matrix of a non-woven fabric (CelliGen™, New Brunswick Scientific, Edison, N J; Wang et al., 1992; Wang et al., 1993; Wang et al., 1994). Briefly described, this reactor comprises an improved reactor for culturing of both anchorage- and non-anchorage-dependent cells. The reactor is designed as a packed bed with a means to provide internal recirculation. Preferably, a fiber matrix carrier is placed in a basket within the reactor vessel. A top and bottom portion of the basket has holes, allowing the medium to flow through the basket. A specially designed impeller provides recirculation of the medium through the space occupied by the fiber matrix for assuring a uniform supply of nutrient and the removal of wastes. This simultaneously assures that a negligible amount of the total cell mass is suspended in the medium. The combination of the basket and the recirculation also provides a bubble-free flow of oxygenated medium through the fiber matrix. The fiber matrix is a non-woven fabric having a “pore” diameter of from 10 μm to 100 μm, providing for a high internal volume with pore volumes corresponding to 1 to 20 times the volumes of individual cells.

The perfused packed-bed reactor offers several advantages. With a fiber matrix carrier, the cells are protected against mechanical stress from agitation and foaming. The free medium flow through the basket provides the cells with optimum regulated levels of oxygen, pH, and nutrients. Products can be continuously removed from the culture and the harvested products are free of cells and can be produced in low-protein medium, which facilitates subsequent purification steps. This technology is explained in detail in WO 94/17178 (Aug. 4, 1994, Freedman et al.), which is hereby incorporated by reference in its entirety.

The Cellcube™ (Corning-Costar) module provides a large styrenic surface area for the immobilization and growth of substrate attached cells. It is an integrally encapsulated sterile single-use device that has a series of parallel culture plates joined to create thin sealed laminar flow spaces between adjacent plates.

The Cellcube™ module has inlet and outlet ports that are diagonally opposite each other and help regulate the flow of media. During the first few days of growth the culture is generally satisfied by the media contained within the system after initial seeding. The amount of time between the initial seeding and the start of the media perfusion is dependent on the density of cells in the seeding inoculum and the cell growth rate. The measurement of nutrient concentration in the circulating media is a good indicator of the status of the culture. When establishing a procedure it may be necessary to monitor the nutrients composition at a variety of different perfusion rates to determine the most economical and productive operating parameters.

Other commercially available perfusion systems include, for example, CellPerj® (Laboratories MABIO International, Tourcoing, France) and the Stovall Flow Cell (Stovall Life Science, Inc., Greensboro, N.C.)

The timing and parameters of the production phase of cultures depends on the type and use of a particular cell line. Many cultures require a different media for production than is required for the growth phase of the culture. The transition from one phase to the other will likely require multiple washing steps in traditional cultures. However, one of the benefits of a perfusion system is the ability to provide a gentle transition between various operating phases. The perfusion system can also facilitate the transition from a growth phase to a static phase induced by an oxygen antagonist. Likewise, the perfusion system can facilitate the transition from a static phase to a growth phase by replacing the solution comprising an oxygen antagonist with, for example, a physiological nutrient media.

G. Catheters

In certain embodiments, a catheter is used to provide a protective agent to an organism. Of particular interest is the administration of such an agent to the heart or vasculature system. Frequently, a catheter is used for this purpose. Yaffe et al., 2004 discusses catheters particularly in the context of suspended animation, though the use of catheters were generally known prior to this publication.

H. Delivery of Gases

1. Respiration System

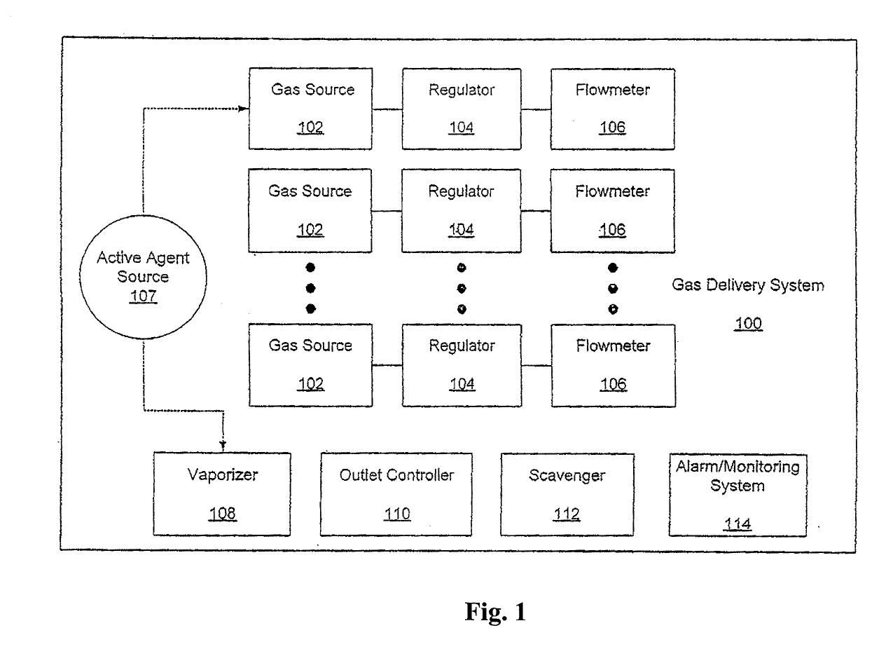

An exemplary gas delivery system 100 is illustrated in FIG. 1. The delivery system 100 is suited for delivering breathable gases, including an active agent, to the respiration system of a subject. The gas delivery system 100 includes one or more gas sources 102. Each of the gas sources 102 is connected to a regulator 104 and a flowmeter 106. The gas delivery system 100 also includes an active agent source 107, an optional vaporizer 108, an outlet controller 110, a scavenger 112, and an alarm/monitoring system 114.

The delivery system 100 may include certain elements generally used in an anesthesia delivery machine. For example, anesthesia delivery machines generally include a high pressure circuit, a low pressure circuit, a breathing circuit, and a scavenging circuit. As described in FIGS. 1-3, one or more of the gas sources 102, the vaporizer 108, the outlet controller 110, the scavenger 112, and/or the alarm/monitoring system 114 may be provided as part of a device having a high pressure, low pressure, breathing, and/or scavenging circuit, and these elements may be similar to those generally used in an anesthesia delivery machine. Anesthesia delivery machines are described, for example, in U.S. Pat. Nos. 4,034,753; 4,266,573; 4,442,856; and 5,568,910, the contents of which are hereby incorporated by reference in their entireties.

The gas sources 102 may be provided by tanks of compressed gas; however, it should be understood that the gas sources 102 can be either a gas or a liquid source that is converted to a gas. For example, the vaporizer 108 can be used to vaporize a liquid gas source. The regulators 104 include valves that reduce the pressure of each of the gas sources 102. The decompressed gas then passes through one of the flowmeters 106, which measures and controls the flow of gas from each of the respective gas sources 102.

The gas sources 102 may be carrier gases that are used to deliver the active agent 107. The carrier gases may be selected to provide a desired environment for a subject to which the active agent from the source 107 is delivered. For example, if the active agent is delivered to a patient as a breathable gas, the carrier gases can include oxygen, nitrous oxide, or air in sufficient quantities to satisfy the needs of the patient. Other inert or active gases may be used.

In some embodiments, one of the gas sources 102 includes the active agent source 107. The active agent from the source 107 may be a liquid gas source that is vaporized by the vaporizer 108 or the active agent may be a gaseous source, such as a compressed gas under high pressure. The active agent can be mixed with one or more of the gas sources 102. The outlet controller 110 controls the amount of the gas mixture that is provided to the subject.

The scavenger 112 is a device or system that scavenges and/or ventilates the gases that are provided to the subject. For example, if the active agent from the source 107 is provided as a breathable gas to a patient, the scavenger 112 can be used to remove the waste gases of the inhalant (such as the active agent), unused oxygen, and exhaled carbon dioxide.

The alarm/monitoring system 114 includes sensors that monitor the gas flow and/or gas content at one or more locations within the delivery system 100. For example, the flow or amount of oxygen may be monitored when the active agent from the source 107 is provided as a breathable gas to a patient to ensure that the carrier gases include sufficient oxygen for the patient. The alarm/monitoring system 114 also includes a user interface that is configured to provide an audio or visual alarm or monitoring information to a user of the delivery system 100, such as a visual display, a light, or audio alarm. The alarm/monitoring system 114 can be configured to notify the user when a predetermined condition is met and/or to provide information regarding gas levels.

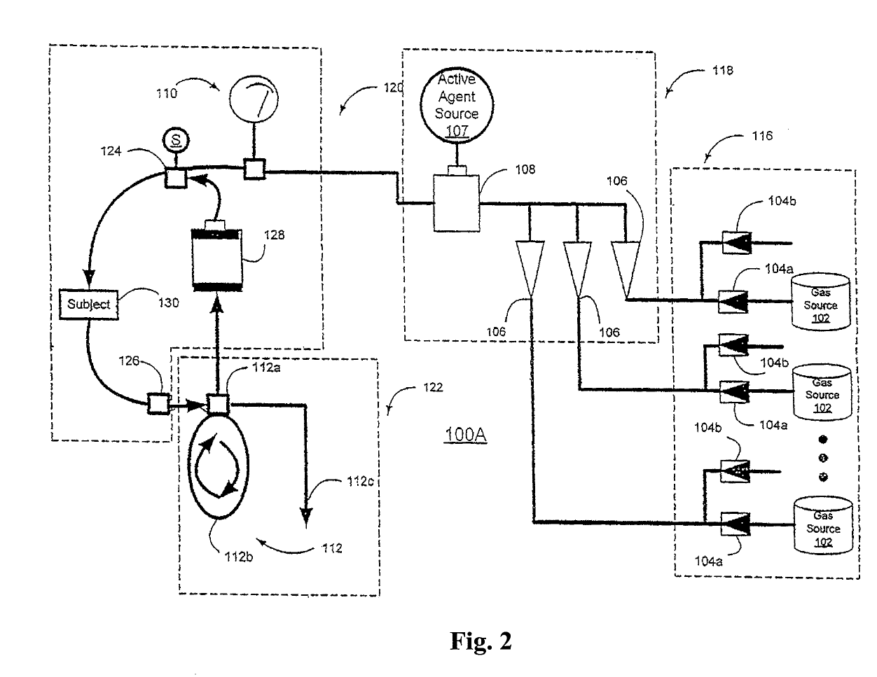

With reference to FIG. 2, a system 100A includes a high pressure circuit 116, a low pressure circuit 118, a breathing circuit 120, and a scavenging circuit 122.

The high pressure circuit 116 includes the compressed gas sources 102, which are connected to regulator valves 104b, 104a. The regulator valves 104a control the amount of gas that flows from each of the gas sources 102, and the regulator valves 104b may be opened to increase the pressure of the gas, for example, by providing an opening to the surrounding atmosphere.

The low pressure circuit 118 includes the flowmeters 106, the active agent source 107, and the vaporizer 108. A gas mixture from the gas sources 102 is provided by the flowmeters 106, which control the amount of each of the gases from the gas sources 102. As illustrated in FIG. 2, the active agent source 107 is a liquid. The active agent source 107 is vaporized by the vaporizer 108 and added to the gas mixture.

The breathing circuit 120 includes the outlet controller 110, two one-way valves 124, 126 and an absorber 128. The scavenger circuit 122 includes a valve 112a, a reservoir 112b, and an outlet 112c. A subject 130 receives the gas mixture from the outlet controller 110 and the resulting gas is ventilated by the scavenger circuit 122. More specifically, the outlet controller 110 controls the amount of the gas mixture that is delivered to the subject 130 via the one-way valve 124. Expired gases flow through the one-way valve 126 to the valve 112a and to the reservoir 112b. Excess gases exit through the outlet 112c of the scavenger 112. Some of the gases may be recycled and flow through the absorber 128 and into the breathing circuit 120. The absorber 128 may be a carbon dioxide absorbing canister for reducing carbon dioxide gases from exhaled gases. In this configuration, expired oxygen and/or active agent may be re-circulated and reused.

One or more sensors S may be added at various positions in the system 10A. The sensors S sense and/or monitor the gases in the system 10A. For example, if one of the gas sources 102 is oxygen, one of the sensors S may be an oxygen sensor configured and positioned to monitor the oxygen in the system 100A so that the patient receives a suitable amount of oxygen. The sensors S are in communication with the alarm/monitoring system 114 (see FIG. 1). If undesirable or dangerous gas levels are present in the system 100, the alarm/monitoring system 114 may alert a user of the system 10A so that appropriate action may be taken, such as increasing the oxygen levels given to the subject 130 or disconnecting the subject 130 from the delivery system 100A.

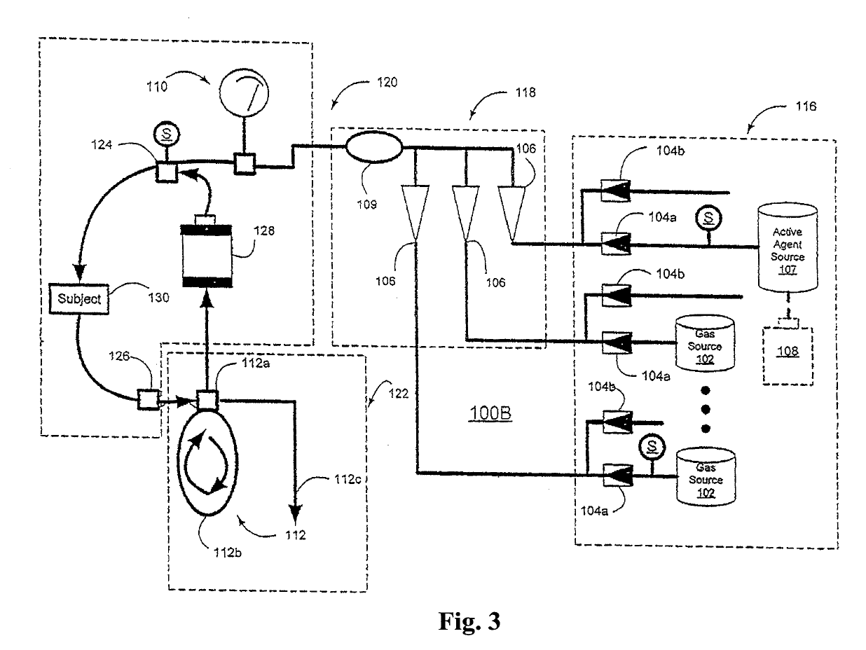

With reference to FIG. 3, a system 100B is shown in which the active agent source 107 is connected to two of the regulator valves 104b, 104a. If the active agent source 107 is a liquid gas source, an optional vaporizer 108 is provided to vaporize the liquid gas source. If the active agent source 107 is gaseous (e.g., a high pressure gas), then the vaporizer 108 may be omitted. The active agent from the source 107 is mixed with the other gas sources 102 in the low pressure circuit 118 in amounts that are controlled by the flowmeters 106. The low pressure circuit 118 includes a gas reservoir 109 that contains any overflow of the gas mixture as it flows to the breathing circuit 120. It should be understood that the active agent source 107 and/or any of the gas sources 102 may be provided as a liquid gas source with a vaporizer. The elements of the system 100B illustrated in FIG. 3 are essentially the same as those described above with respect to FIG. 2 and will not be described further.

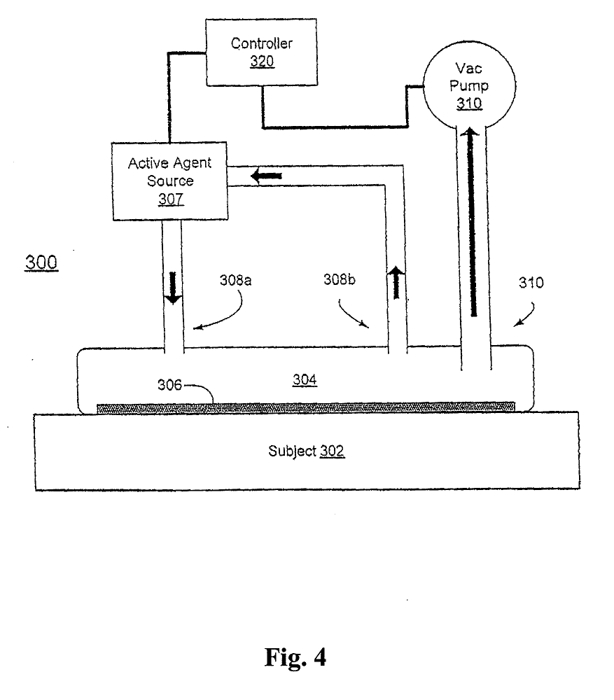

Methods according to embodiments of the present invention which may be carried out using the systems 100, 100A, 100B are illustrated in FIG. 4. A mixture of one or more breathable gas sources is provided (Block 202). The breathable gas sources may be obtained from the gas sources 102 as described with respect to FIGS. 1-3. A predetermined amount of the active agent is added to the gas mixture (Block 204), such as is shown with respect to the active agent source 107 in FIGS. 1-3. The gas mixture is administered to the subject 120 (Block 306). Exhaled gases are ventilated and/or recycled (Block 208), for example, by the scavenger 112. Although the methods of FIG. 4 are described with respect to the systems 100, 100A, 100B of FIG. 1-3, it should be understood that any suitable system or device may be used to carry out the steps in FIG. 4.

2. Reduced Pressure Delivery System

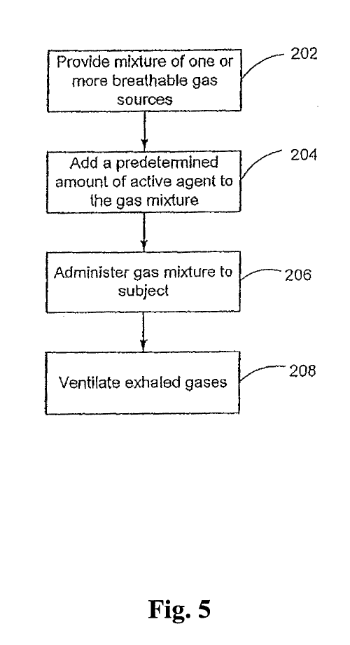

Embodiments of a gas delivery system 300 are illustrated with respect to FIG. 5. The gas delivery system 300 is positioned on a subject 302. The gas delivery system 300 is particularly suited to deliver an active agent in a gas mixture to the tissue of a subject 302, for example, wound tissue.

The system 300 includes a reduced pressure chamber 304 having a screen 306 that covers the treatment area of the subject 302. The reduced pressure chamber 304 is connected to a vacuum pump 310 by a pump outlet 310a. The reduced pressure chamber 304 includes an inlet 308a and an outlet 308b, which are in turn connected to an active agent source 307. A controller 320 is connected to the active agent source 307 and the vacuum pump 310. Reduced pressure chambers and vacuum pump systems are discussed in U.S. Pat. Nos. 5,645,081 and 5,636,643, the contents of which are hereby incorporated by reference in their entireties.

The reduced pressure chamber 304 is configured to enclose an area of the subject 302 to provide a fluid-tight or gas-tight enclosure to effect treatment of the area with reduced or negative pressure and the active agent source 307. The pressure chamber 304 can be affixed to the subject 302 with a cover (not shown), such as a flexible, adhesive, fluid impermeable polymer sheet. The cover can have an adhesive backing that functions to cover the skin around the periphery of the area being treated and to provide a generally gas-tight or fluid-tight seal and to hold the chamber 304 in position.

The screen 306 is positioned over the treatment area of the subject 302. For example, if the treatment area of the subject 302 includes a wound, the screen 306 can be positioned over the wound to prevent its overgrowth. The size and configuration of the screen 306 can be adjusted to fit the individual treatment area, and may be formed from a variety of porous materials. The material should be sufficiently porous to allow oxygen any other gases, such as gases from the active agent source 307, to reach the treatment area. For example, the screen 306 can be in the form of an open-cell polymer foam, such as a polyurethane foam, which is sufficiently porous to allow gas flow to and/or from the treatment area. Foams may be used that vary in thickness and rigidity, although it may be desirable to use a spongy material for the patient's comfort if the patient must lie upon the appliance during treatment. The foam may also be perforated to enhance gas flow and to reduce the weight of the system 300. The screen 306 may be cut to an appropriate shape and size to fit within the treatment area, or alternatively, the screen 306 may be sufficiently large to overlap the surrounding skin.

The vacuum pump 310 provides a source of suction within the reduced pressure chamber 304. The active agent source 307 provides an amount of the active agent to the reduced pressure chamber 304. The controller 320 controls the amount of vacuum applied to the reduced pressure chamber 304 by the vacuum pump 310 and the amount of the active agent that is supplied to the chamber 304 by the active agent source 307.

It should be understood that the controller 320 can apply a vacuum and/or the active agent in a substantially constant manner, cyclically, or using various fluctuations or patterns or any combination thereof. In some embodiments, the active agent is supplied by the active agent source 307 alternatively with the vacuum pumping action of the vacuum pump 310. That is, the controller 320 alternatively activates the vacuum pump 310 while deactivating the active agent source 307 and then activates the active agent source 307 while deactivating the vacuum pump 310. The pressure in the reduced pressure chamber 304 is allowed to fluctuate. In other embodiments, a substantially constant pressure is maintained by the vacuum pump 310 and the active agent source 307 provides a substantially constant amount of active agent to the chamber 304 in the reduced pressure environment. In some embodiments, a substantially constant pressure is maintained by the vacuum pump 310 and the amount of the active agent varies in a cyclical manner. In other embodiments, the pressure in the reduced pressure chamber 304 is made to fluctuate by the vacuum pump 310, and the amount of active agent supplied by the source 307 also fluctuates. The fluctuations of either the vacuum pump 310 and the resulting pressure in the chamber 304 or the amount of active agent supplied by the source 307 may be cyclical or not cyclical.

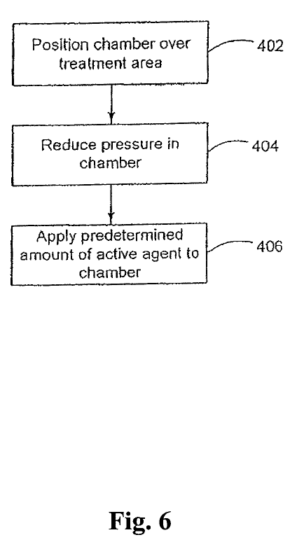

Other methods according to embodiments of the present invention that may be carried out using the system 300 are illustrated in FIG. 6. The chamber 304 is positioned over the treatment area of the subject 302 (Block 402). Pressure is reduced in the chamber 304 by the vacuum pump 310 (Block 404). A predetermined amount of active agent from the active agent source 307 is applied to the chamber (Block 406). Although the methods of FIG. 6 are described with respect to the system 300 of FIG. 4, it should be understood that any suitable system or device may be used to carry out the steps in FIG. 6. For example, the outlet 308b may be omitted and the active agent may be supplied to the chamber 304 by the single inlet 308a. Other gases may also be added to the chamber 304, for example, using a single inlet or an inlet and an outlet, such as is illustrated with K; respect to the active agent source 307 and the inlet 308a and the outlet 308b. In some embodiments, the vacuum pump 310 is attached to an additional collection container between the pump 310 and the chamber 304 for collecting exudates from the treatment area, for example, as described in U.S. Pat. No. 5,636,643.

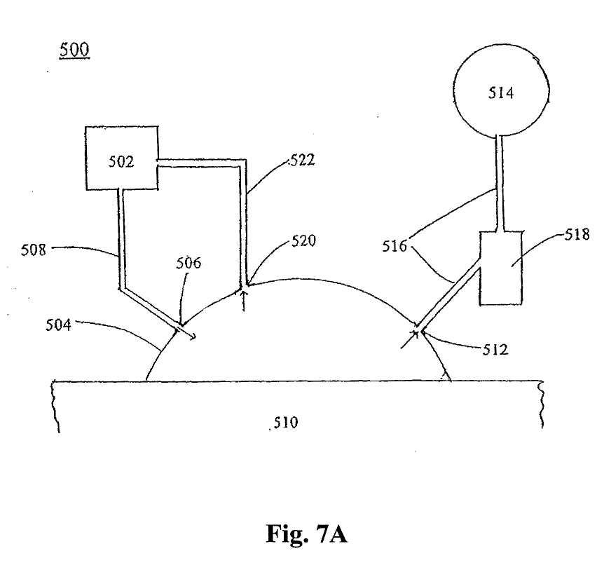

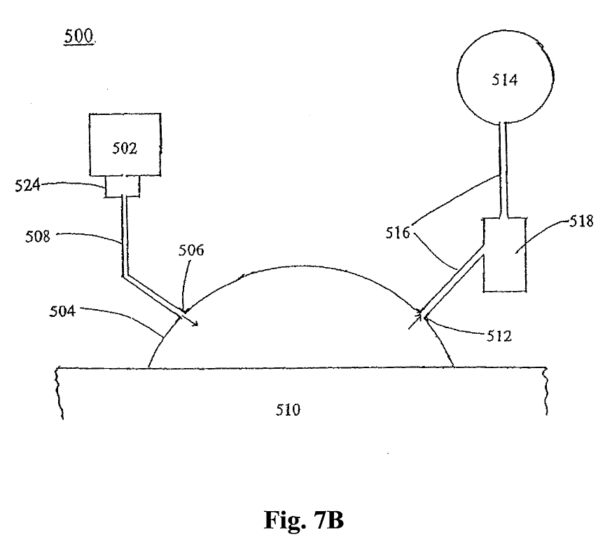

In some embodiments, the negative pressure gas delivery system 500, as depicted in FIG. 7A, comprises an active oxygen antagonist source in a container 502, connected to a drape 504, via an inlet 506, by a conduit 508. The drape forms a sealed envelope against a tissue site 510, which may a wound site. In some embodiments, the drape has an outlet 512 in communication with a negative pressure source 514, via a conduit 516. In some embodiments a waste canister 518, which may be a removable waste canister, is in communication between the outlet and the negative pressure source. In some embodiments, a return outlet 520 is connection with the container 502 via a conduit 522. In some embodiments, as shown in FIG. 7B, a vaporizer 524 is interposed in the communication between the container 502 and the drape 504.

The conduits may be flexible and may suitably be plastic of a like material hose. The negative pressure source 514, which may suitably be a vacuum pump, is in some embodiments in fluid communication with the outlet 512 via the conduit 516, for the promotion of fluid drainage, as is known in the art. In some embodiments, the waste canister 518 is placed under vacuum through fluid communication to collect drainage fluid. Preferably a filter (not shown), which may be a hydrophobic membrane filter, is interposed between the canister the negative pressure source to protect against contamination from drainage fluids sucked into the canister. In some embodiments, the drape 504 comprises an elastomeric material, which may therefore accommodate pressure changes over the tissue site area during intermittent operation of the negative pressure source. In some embodiments, the periphery of the drape is covered with a pressure sensitive adhesive, which may be acrylic adhesive, for sealing the drape over the tissue site.

Negative pressure gas delivery systems 300 and 500 as illustrated in FIG. 4 and FIG. 7A-B are useful for treating a variety of areas for treatment, and, in particular, for treating wounds. Wounds that may be treated using the system 300 include infected open wounds, decubitus ulcers, dehisced incisions, partial thickness burns, and various lesions to which flaps or grafts have been attached. Treatment of a wound can be carried out by securing a gas delivery system to the treatment site as previously shown and described, maintaining a substantially continuous or cyclical reduced pressure within the reduced pressure chamber 304 and supplying the active agent to the chamber 304 in a substantially continuous or cyclical fashion until the wound has reached a desired improved condition. A selected state of improved condition may include formation of granulation tissue sufficient for the attachment of a flap or graft, reduction of microbial infection in the wound, arrest or reversal of burn penetration, closure of the wound, integration of a flap or graft with the underlying wounded tissue, complete healing of the wound, or other stages of improvement or healing appropriate to a given type of wound or wound complex. The gas delivery system may be changed periodically, such as at 48 hrs intervals, during treatment, particularly when using a gas delivery system incorporating a screen on or in the wound. The method may be practiced using a negative or reduced pressure ranging from 0.01 to 0.99 atmospheres, or the method may be practiced using a negative or reduced pressure ranging between 0.5 to 0.8 atmospheres. The time period for use of the method on a wound may be at least 12 hrs, but can be, for example, extended for one or more days. There is no upper limit beyond which use of the method is no longer beneficial; the method can increase the rate of closure up to the time the wound actually closes. Satisfactory treatment of various types of wounds may be obtained via the use of reduced pressures equivalent to about 2 to 7 in. Hg below atmospheric pressure.

Supplying reduced pressure to the gas delivery system in an intermittent or cyclic manner, such as described above, may be useful for treating wounds in the presence of the active agent. Intermittent or cyclic supply of reduced pressure to a gas delivery system may be achieved by manual or automatic control of the vacuum system. A cycle ratio, the ratio of “on” time to “off” time, in such an intermittent reduced pressure treatment may be as low as 1:10 or as high as 10:1. A typical ratio is approximately 1:1 which is usually accomplished in alternating 5 minute intervals of reduced pressure supply and non-supply.

A suitable vacuum system includes any suction pump capable of providing at least 0.1 pounds of suction to the wound, or up to three pounds suction, or up to fourteen H (14) pounds suction. The pump can be any ordinary suction pump suitable for medical purposes that is capable of providing the necessary suction. The dimension of the tubing interconnecting the pump and the reduced pressure appliance is controlled by the pump's ability to provide the suction level needed for operation. A ¼ inch diameter tube may be suitable.

Embodiments of the present invention also include methods of treating damaged tissue which include the steps of applying negative pressure to a wound and the active agent for a selected time and at a selected magnitude sufficient to reduce bacterial density in the wound. Open wounds are almost always contaminated with harmful bacteria. Generally a bacterial density of 10⁵ bacterial organisms per gram of tissue is regarded as infected. It is generally accepted that at this level of infection, grafted tissue will not adhere to a wound. These bacteria must be killed, either through the wound host's natural immune response or through some external method, before a wound will close. The application of negative pressure and active agent to a wound may reduce the bacterial density of the wound. It is believed that this effect may be due to the bacteria's incompatibility with a negative pressure environment or the increased blood flow to the wound area in combination with exposure to the active agent as blood brings with it cells and enzymes to destroy the bacteria. Methods according to embodiments of the present invention can be used to reduce bacterial density in a wound by at least half. In some embodiments, it can be used to reduce bacterial density by at least 1.000-fold or by at least 1,000,000-fold.

Embodiments of the present invention also include methods of treating a burn which include the steps of applying negative pressure and the active agent to the burn over an area with predetermined reduced pressure and for a time sufficient to inhibit formation of a full thickness burn. A partial thickness burn, one which has a surface layer of dead tissue and an underlying zone of stasis, is often sufficiently infected so that it will transform within 24-48 hrs into a full thickness burn, one in which all epidermal structures are destroyed. The application of negative pressure and an amount of the active agent to the wound may prevent the infection from becoming sufficiently severe to cause destruction of the underlying epidermal structures. The magnitude, pattern, and duration of pressure application can vary with the individual wound.

Embodiments of the present invention also include methods for enhancing the attachment of living tissue to a wound which comprises the steps of first joining the living tissue to the wound to form a wound-tissue complex, then applying a negative or reduced pressure of selected magnitude and an amount of the active agent to the wound-tissue complex over an area sufficient to promote migration of epithelia and subcutaneous tissue toward the complex, with the negative pressure and exposure to the active agent being maintained for a selected time period sufficient to facilitate closure of the wound. Attachment of living tissue to a wound is a common procedure that can take many forms. For example, one common technique is the use of a “flap,” a technique in which skin tissue from an area adjacent to the wound is detached on three sides but remains attached on the fourth, then is moved onto the wound. Another frequently used technique is an open skin graft in which skin is fully detached from another skin surface and grafted onto the wound. The application of negative pressure and active agent to the wound-graft complex reduces bacterial density in the complex and improves blood flow to the wound, thereby improving the attachment of the grafted tissue.

3. Sequential or Simultaneous Administration of One or More Gases

As described above, certain embodiments of the present invention comprise the administration of one or more gases to biological matter, such as a subject. As described, the gas(es) administered may consist entirely of one or more active agents, or may be a carrier gas(es) for at least one active agent. The methods now described provide additional means by which one or more such gases may be provided to biological matter, such as a subject.

In certain embodiments, automated administration of one or more gases to biological matter, such as a subject, is contemplated. Methods of and devices for such automated administration of one or more gases are well-known to those of ordinary skill in the art. See, e.g., U.S. Pat. Nos. 5,692,497, 6,109,260, 6,164,276 and 6,962,154; and U.S. Pat. Appln. No. 2005/0217667, each of which is incorporated by reference in its entirety. Such automated gas delivery devices—“gas blenders,” as used herein (also called gas mixers)—can also be manually operated. Methods of automation include, for example, programmable mechanisms by which a user may enter into the device a program of gas delivery. Gas blenders are well-known to those of skill in the art. See, e.g., Columbus Instruments (Columbus, Ohio) Pegas 4000MF Gas Mixer; Kojuma Instruments (Tokyo, Japan) Kofloc Gas Blender Series Model 3610; U.S. Pat. Nos. 5,887,611 and 6,857,443, incorporated herein in its entirety.

Face/Nose masks that may be used for the automated delivery of one or more gases, or a mixture of gases, to a human subject are known to those of ordinary skill in the art. For example, U.S. Pat. Nos. 4,354,488 and 5,474,060, each of which is incorporated by reference in its entirety, describe such masks.

I. Other Apparatuses

Within certain embodiments of the invention, it may be desirable to supplement the methods of the present invention for the treatment of patients who will be or have been subjected to trauma with the ability to externally manipulate the core body temperature of the patient. In this regard, the core body temperature of a patient may be, in combination with the methods of the present invention, manipulated by invasive or non-invasive routes. Invasive methods for the manipulation of core body temperature include, for example, the use of a heart-lung pump to heat or cool the patient's blood thus raising or cooling the patient's core body temperature. Non-invasive routes to manipulate core body temperature include systems and apparatuses that transfer heat into or out of the patient's body.

J. Further Delivery Devices or Apparatuses

In some embodiments it is contemplated that methods or compositions will involve a specific delivery device or apparatus. Any method discussed herein can be implemented with any device for delivery or administration including, but not limited, to those discussed herein.

For topical administration of active compounds of the invention may be formulated as solutions, gels, ointments, creams, suspensions, etc. as are well-known in the art. Systemic formulations may include those designed for administration by injection or infusion, e.g., subcutaneous, intravenous, intramuscular, intrathecal or intraperitoneal injection, as well as those designed for transdermal, transmucosal, oral or pulmonary administration.

For oral administration, the active compounds of the invention to be formulated as tablets, pills, dragees, capsules, liquids, gels, syrups, slurries, suspensions and the like, for oral ingestion by a patient to be treated or oral liquid preparations such as, for example, suspensions, elixirs and solutions.

For buccal administration, the compositions may take the form of tablets, lozenges, etc. formulated in conventional manner. Other intramucosal delivery might be by suppository or intranasally.

For administration directly to the lung by inhalation the compound of invention may be conveniently delivered to the lung by a number of different devices. For example,

Metered-Dose Inhalers (MIDs): a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas may be used to deliver the compound of invention directly to the lung. MDI devices are available from a number of suppliers such as 3M Corporation (e.g., on the world wide web at 3m.com/us/healthcare/manufacturers/dds/pdf/idd_valve_canister_brochure.pdf-), Nasacort from Aventis (e.g., world wide web at products.sanofi-aventis.us/Nasacort_HFA/nasacort_HFA.html-63k-), Boehringer Ingelheim, (e.g., world wide web at.boehringer-ingelheim.com/corporate/home/download/r_and_d2003.pdf) Aerobid from Forest Laboratories, (e.g., world wide web at.frx.com/products/aerobid.aspx) Glaxo-Wellcome, (for example, on the world wide web at .gsk.com/research/newmedicines/newmedicines_pharma.html) and Schering Plough, (world wide web at .schering-plough. com/schering_plough/pc/allergy_respiratory.jsp).

Dry Powder Inhalers (DPIs): DPI devices typically use a mechanism such as a burst of gas to create a cloud of dry powder inside a container, which may then be inhaled by the patient. DPI devices are also well known in the art and may be purchased from a number of vendors which include, for example, Foradil aerolizer from Schering Corporation, (e.g., world wide web .spfiles.com/piforadil.pdf) Advair Diskus from Glaxo-Wellcome. (e.g., world wide web at us.gsk.com/products/assets/us_advair.pdf-) A popular variation is the multiple dose DPI (“MDDPI”) system, which allows for the delivery of more than one therapeutic dose. MDDPI devices are available from companies such as Plumicort Turbuhaler from AstraZeneca, (e.g, world wide web at.twistclickinhale.com/GlaxoWellcome, (e.g., world wide web at us.gsk.com/products/assets/us_advair.pdf-) and Schering Plough, (e.g., world wide web at .schering-plough.com/schering_plough/pc/allergy_respiratory.jsp). It is further contemplated that such devices, or any other devices discussed herein, may be altered for single use.

Electrohydrodynamic (EHD) aerosol delivery: EHD aerosol devices use electrical energy to aerosolize liquid drug solutions or suspensions (see, e.g., Noakes et al., U.S. Pat. No. 4,765,539; Coffee, U.S. Pat. No. 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807). EHD aerosol devices may more efficiently deliver drugs to the lung than existing pulmonary delivery technologies.

Nebulizers: Nebulizers create aerosols from liquid drug formulations by using, for example, ultrasonic energy to form fine particles that may be readily inhaled. Examples of nebulizers include devices supplied by Sheffield/Systemic Pulmonary Delivery Ltd. (see, e.g., U.S. Pat. Nos. 5,954,047; 5,950,619; 5,970,974); and Intal nebulizer solution by Aventis, (e.g., world wide web at .fda.gov/medwatch/SAFETY/2004/feb_PI/Intal_Nebulizer_PI.pdf).

For administration of a gas directly to the lungs by inhalation various delivery methods currently available in the market for delivering oxygen may be used. For example, a resuscitator such as an ambu-bag may be employed (see, e.g., U.S. Pat. Nos. 5,988,162 and 4,790,327). An ambu-bag consists of a flexible squeeze bag attached to a face mask, which is used by the physician to introduce air/gas into the casualty's lungs.

A portable, handheld medicine delivery device capable producing atomized agents that are adapted to be inhaled through a nebulizer by a patient suffering from a respiratory condition. In addition, such delivery device provides a means wherein the dose of the inhaled agent can be remotely monitored and, if required altered, by a physician or doctor. See U.S. Pat. No. 7,013,894. Delivery of the compound of invention may be accomplished by a method for the delivery of supplemental gas to a person combined with the monitoring of the ventilation of the person with both being accomplished without the use of a sealed face mask such as described in U.S. Pat. No. 6,938,619. A pneumatic oxygen conserving device for efficiently dispensing oxygen or other gas used during respiratory therapy such that only the first part of the patient's breath contains the oxygen or other therapeutic gas, (See U.S. Pat. No. 6,484,721). A gas delivery device is used which is triggered when the patient begins to inhale. A tail of gas flow is delivered to the patient after the initial inhalation timed period to prevent pulsing of gas delivery to the patient. In this manner gas is only delivered to the patient during the first portion of inhalation preventing gas from being delivered which will only fill the air passageways to the patient's lungs. By efficiently using the oxygen, cylinder bottles of oxygen used when a patient is mobile will last longer and be smaller and easier to transport. By pneumatically delivering the gas to the patient no batteries or electronics are used.

Electronic means of delivering gases are also contemplated. Nebulizers, equipment and methods for supplying doses of at least one gas carrying particles of one or more active products via electronically controlled or programmed methods are described in U.S. Pat. No. 5,560,353, incorporated herein by reference.

All the devices described here may have an exhaust system to bind or neutralize the compound of invention.

Transdermal administration of the compound of the invention can be achieved by medicated device or patch which is affixed to the skin of a patient. The patch allows a medicinal compound contained within the patch to be absorbed through the skin layers and into the patient's blood stream. Such patches are commercially available as Nicoderm CQ patch from Glaxo Smithkline, (world wide web at nicodermcq.com/NicodermCQ.aspx) and as Ortho Evra from Ortho-McNeil Pharmaceuticals, (world wide web at ortho-mcneilpharmaceutical.com/healthinfo/womenshealth/products/orthoevra.html). Transdermal drug delivery reduces the pain associated with drug injections and intravenous drug administration, as well as the risk of infection associated with these techniques. Transdermal drug delivery also avoids gastrointestinal metabolism of administered drugs, reduces the elimination of drugs by the liver, and provides a sustained release of the administered drug. Transdermal drug delivery also enhances patient compliance with a drug regimen because of the relative ease of administration and the sustained release of the drug.

Other modifications of the patch include the Ultrasonic patch which is designed with materials to enable the transmission of ultrasound through the patch, effecting the delivery of medications stored within the patch, and to be used in conjunction with ultrasonic drug delivery processes (see, e.g., U.S. Pat. No. 6,908,448). Patch in a bottle (U.S. Pat. No. 6,958,154) includes a fluid composition, e.g., an aerosol spray in some embodiments, that is applied onto a surface as a fluid, but subsequently dries to form a covering element, such as a patch, on a surface of a host. The covering element so formed has a tack free outer surface covering and an underlying tacky surface that helps adhere the patch to the substrate.

Another drug delivery system comprises one or more ball semiconductor aggregations and facilitating release of a drug stored in a reservoir. The first aggregate is used for sensing and memory, and a second aggregation for control aspects, such as for pumping and dispensing of the drug. The system may communicate with a remote control system, or operate independently on local power over a long period for delivery of the drug based upon a request of the patient, timed-release under control by the system, or delivery in accordance with measured markers. See, e.g., U.S. Pat. No. 6,464,687.

PUMPS and Infusion Devices: An infusion pump or perfusor infuses fluids, medication or nutrients into a patient's circulatory system. Infusion pumps can administer fluids in very reliable and inexpensive ways. For example, they can administer as little as 0.1 mL per hour injections (too small for a drip), injections every minute, injections with repeated boluses requested by the patient, up to maximum number per hour (e.g. in patient-controlled analgesia), or fluids whose volumes vary by the time of day. Various types of infusion devices have been described in the following patent applications before the United States Patent and Trademark Office. These include but are not limited to U.S. Pat. No. 7,029,455, U.S. Pat. No. 6,805,693, U.S. Pat. No. 6,800,096, U.S. Pat. No. 6,764,472, U.S. Pat. No. 6,742,992, U.S. Pat. No. 6,589,229, U.S. Pat. No. 6,626,329, U.S. Pat. No. 6,355,019, U.S. Pat. No. 6,328,712, U.S. Pat. No. 6,213,738, U.S. Pat. No. 6,213,723, U.S. Pat. No. 6,195,887, U.S. Pat. No. 6,123,524 and U.S. Pat. No. 7,022,107. In addition, infusion pumps are also available from Baxter International Inc. (world wide web at .baxter.com/products/medication_management/infusion_pumps/), Alaris Medical Systems (world wide web at alarismed.com/products/infusion.shtml) and from B Braun Medical Inc. (world wide web at bbraunusa.com/index.cfln?uuid=001 AA837D0B759A1E34666434FF604ED).

Oxygen/Gas bolus delivery device: Such a device for delivering gas to Chronic Obstructive Pulmonary Disease (COPD) patients is a available from Tyco Healthcare (world wide web at tycohealth-ece.com/files/d0004/ty_zt7ph2.pdf). It can also be used to deliver the compound of invention. The above device is cost-effective, lightweight, inconspicuous and portable.

“Patch in a bottle” (U.S. Pat. No. 6,958,154) includes a fluid composition, e.g., an aerosol spray in some embodiments, that is applied onto a surface as a fluid, but subsequently dries to form a covering element, such as a patch, on a surface of a host. The covering element so formed has a tack free outer surface covering and an underlying tacky surface that helps adhere the patch to the substrate.

Implantable Drug Delivery System: Another drug delivery system comprises one or more ball semiconductor aggregations and facilitating release of a drug stored in a reservoir. The first aggregate is used for sensing and memory, and a second aggregation for control aspects, such as for pumping and dispensing of the drug. The system may communicate with a remote control system, or operate independently on local power over a long period for delivery of the drug based upon a request of the patient, timed-release under control by the system, or delivery in accordance with measured markers. See U.S. Pat. No. 6,464,687.

The contents of each of the cited patents and web addresses discussed in this section are hereby incorporated by reference.

### VI. Combination Therapies

The compounds and methods of the present invention may be used in the context of a number of therapeutic and diagnostic applications. In order to increase the effectiveness of a treatment with the compositions of the present invention, such as oxygen antagonists, it may be desirable to combine these compositions with other agents effective in the treatment of those diseases and conditions (secondary therapy). For example, the treatment of hemorrhagic shock may involve one or more of the following: agent to increase pressure in the arteries and increase cardiac output (e.g., dopamine, dobutamine, norepinephrine); blood vessel dilators or constrictors; corticosteroids; antibiotics; morphine; and thrombolytic therapy (e.g., tissue plasminogen activator, Streptokinase (SK), Reteplase, Tenecteplase, Urokinase, Lanoteplase, and Staphylokinase).

Various combinations may be employed; for example, an oxygen antagonist, such as H₂S, is “A” and the secondary therapy is “B”:

| | | | | | |
|---|---|---|---|---|---|
| A/B/A | B/A/B B/B/A | A/A/B | A/B/B B/A/A | A/B/B/B | B/A/B/B |
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the active compounds of the Spresent invention to biological matter will follow general protocols for the administration of that particular secondary therapy, taking into account the toxicity, if any, of the active compound treatment. It is expected that the treatment cycles would be repeated as necessary. It also is contemplated that various standard therapies, as well as surgical intervention, may be applied in combination with the described therapies.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Hydrogen Sulfide Protects Against Lethal Hemorrhage

To determine whether H₂S treatment could be used to reduce morbidity and/or tissue damage associated with a more clinically relevant acute injury model of ischemic hypoxia, rats were treated with H₂S during controlled lethal hemorrhage, which reduces oxygen supply to tissues and results in death (Blackstone et al., 2005). In this study, rats treated with H₂S survived lethal blood loss and fully recovered.

Rats were treated with H₂S during controlled lethal hemorrhage (60% blood loss). After surgical implantation of catheters and recovery, blood was removed from conscious animals in 40 minutes. A small amount (300 ppm) of H₂S mixed with room air was administered to treated animals twenty minutes after the beginning of the bleed (i.e., after 30% blood loss). Animals were returned to room air without H₂S at the end of the bleed. Three hours after the end of the bleed, surviving animals were given one shed-blood volume of lactated ringers solution intravenously.

Most (6/7) of the H₂S treated rats survived hemorrhage and 3 hour shock period and recovered completely (Table 1). None of these surviving rats exhibited behavioral or functional defects after recovery. One H₂S treated animal died 174 minutes after the end of the bleed. All of the untreated animals died within 82 minutes after the end of the bleed; average survival time of untreated animals was 35+/−26 minutes. Using a two-tailed Fishers exact T-test, the p value is 0.0047.

In the first twenty minutes of bleeding (before 30% blood loss) rats increased respiration rate and tidal volume to compensate for decreased oxygen carrying capacity due to blood loss. This increase in ventilation resulted in a decreased respiratory carbon dioxide production (V_{CO2}) (Table 1). After 60% blood loss, both H₂O treated and untreated animals exhibited decreased V_{CO2}. Arterial blood lactate increased while pCO₂, bicarbonate ([HCO₃⁻]), pH, and base excess decreased (Table 1). Thus hemorrhage resulted in metabolic acidosis with respiratory compensation. However, in H₂S treated rats, these changes were smaller in magnitude representing a decrease in metabolic acidosis. Furthermore, in H₂S treated animals, V_{CO2} did not continue to decrease after hemorrhage. In untreated animals, V_{CO2} decreased steadily until the animals stopped breathing. H₂S administration appears to prevent the shock response from progressing to death.

TABLE 1
Survival and physiology of a rat hemorrhage model using H₂S

| | |
|---|---|
| H₂S Treated | Untreated |

Survival

| | | |
|---|---|---|
| Complete recovery | 85.7% (6/7) | 0% (0/7) |
| Time to death of non-survivors (min) | 174 (1/7) | 35 +/− 26 |

CO₂ production (V_{CO2}) in ml/kg/min:

| | | |
|---|---|---|
| Pre-bleed | 25 +/− 4 | 26 +/− 6 |
| Mid-bleed | 20 +/− 2 | 21 +/− 3 |
| End of bleed | 16 +/− 2 | 11 +/− 3 |
| 15 minutes post bleed | 17 +/− 3 | 7 +/− 5 |

Blood CO₂ content (pCO₂) in mmHg

| | | |
|---|---|---|
| Pre-bleed | 45 +/− 6 | 44 +/− 3 |
| End of bleed | 35 +/− 6 | 21 +/− 3 |

Blood bicarbonate content ([HCO₃⁻]) in mmol/L

| | | |
|---|---|---|
| Pre-bleed | 32 +/− 3 | 30 +/− 1 |
| End of bleed | 21 +/− 3 | 12 +/− 3 |

Blood pH

| | | |
|---|---|---|
| Pre-bleed | 7.46 +/− 0.03 | 7.45 +/− 0.02 |
| End of bleed | 7.41 +/− 0.02 | 7.35 +/− 0.06 |

Blood base excess in mmol/L

| | | |
|---|---|---|
| Pre-bleed | 8 +/− 2 | 6 +/− 1 |
| End of bleed | −5 +/− 4 | −14 +/− 3 |

Blood Lactate in mmol/L

| | | |
|---|---|---|
| Pre-bleed | 1.4 +/− 0.5 | 1.2 +/− 0.2 |
| End of bleed | 6.6 +/− 1 | 11 +/− 3 |

### Example 2

### Benefit of Short-Term Exposure to Hydrogen Sulfide during Hemorrhage

Male Sprague Dawley rats weighing 275-350 grams were purchased from Charles River Laboratories one week before each experiment. On the day of the experiment, catheters were surgically implanted in right femoral artery and vein. Catheters exited behind scapulae.

Rats were administered buprenorphine and allowed to recover. 80-100 units of heparin was administered intravenously. Conscious unrestrained rats were placed in a 2.75 liter crystallization dish with a glass lid. Room air, room air with hydrogen sulfide (test animals), and room air containing nitrogen (control animals) was supplied at 3 liters per minute using a thermal mass flow controller (Sierra Instruments). Catheters, temperature probe, and gas sampling tube were passed through a hole drilled in the middle of the lid. Temperature was maintained at approximately isothermal temperature (27+/−2° C.).

Blood was removed using a peristaltic pump at a rate that resulted in completion of the bleed within 40 minutes. Blood volume was calculated using the following equation (0.06×body mass)+0.77 (Lee et al., 1985). Blood was weighed as it was removed. A source tank of H₂S (20,000 ppm balanced with nitrogen) was purchased from Byrne Specialty Gas. Hydrogen sulfide was diluted into room air to a concentration of 2000 ppm using a thermal mass flow controller and delivered to the animals at 3 liters per minute.

Twenty minutes (half way) into the constant rate bleed test animals were exposed to room air containing 2000 ppm hydrogen sulfide. The exposure was terminated when animals exhibited apnea and dystonia. The length of exposure was between 1 and 2 minutes. The maximum concentration in the chamber is estimated to be between 1000 and 1500 ppm. As soon as apnea and dystonia were observed the animals were exposed to room air. Animals resumed regular breathing patterns within 20 to 30 seconds. Control animals were exposed to the same conditions but without hydrogen sulfide. Control animals did not exhibit apnea or dystonia.

Metabolic rate was determined by measuring CO₂ production using a Licor Li7000. Temperature and CO₂ data were collected using an ADI PowerLab. Arterial blood values were measured with I-Stat blood chemistry analyze. Time of death was declared when animals stopped breathing and CO₂ production ceased. Surviving rats were given lactated ringers ad libitum 3 hours after the end of the bleed. After resuscitation, rats were transferred to clean cages with food and water and housed at 30° C. for approximately 16 hours. Catheters were surgically removed and animals were allowed to recover for several hours at 30° C. before being transferred back to the colony. Behavior and function tests were selected from a battery of tests described in the SHIRPA protocol (Rogers et al., 1997).

In these experiments 7 out of 8 of the hydrogen sulfide animals survived the treatment. Of the two control animals tested both died.

### Prophetic Example 3

### Hydrogen Sulfide Dose Selection in Humans

Hydrogen sulfide can be administered to an animal or human to induce stasis by any of a number of dosage forms and routes of administration, including, but not limited to, inhalation of the gaseous form or intravenous administration of a solution of hydrogen sulfide. A method to determine the dosage form and route of administration of hydrogen sulfide sufficient to induce stasis in a whole organism in need of stasis is described. A test organism (e.g., a rat, dog, pig, monkey) is exposed to increasing concentrations of hydrogen sulfide administered either as bolus doses, intermittently, or continuously, and the physiological state, including but not limited to, core body temperature, oxygen consumption, carbon dioxide production, heart rate, blood pressure, breathing rate, blood pH, movement, and wakefulness are monitored while at various timepoints blood samples (0.5 mL) are removed. Concentrations of hydrogen sulfide that are present in the test animals' blood-derived plasma are measured using methods known in the art, including, but not limited to X derivatization, Y extraction, and quantitation using gas chromatography and mass spectrometry.

Correlation of the steady state plasma levels of hydrogen sulfide engendered by a particular dosing regimen in the test animal with the achievement of stasis, to varying degrees, in the test animal, defines an effective dose of hydrogen sulfide sufficient to induce stasis in the test animal. The effective dose for inducing stasis in a human in need of stasis is determined by identifying the dose, route of administration, and dosing regimen of hydrogen sulfide that achieves the same steady state plasma concentrations of hydrogen sulfide in the humans as are achieved in the test animals under conditions where stasis is induced. The effective concentration of hydrogen sulfide to achieve stasis in a human depends on the dosage form and route of administration. For inhalation, in some embodiments effective concentrations are in the range of 50 ppm to 500 ppm, delivered continuously. For intravenous administration, in some embodiments effective concentrations are in the range of 0.5 to 50 milligrams per kilogram of body weight delivered continuously.

The range in each case is characterized by increasing degrees of stasis achieved with increasing dose of the hydrogen sulfide. A dose of hydrogen sulfide sufficient to cause a sustained, 12-24 hour drop of three to five degrees Celsius to 32-34 degrees Celsius in the core body temperature of a human who has suffered out-of-hospital cardiac arrest and who is unconscious upon resuscitation and resumption of a heartbeat is predicted to have a significant survival advantage over a similar human not exposed to hydrogen sulfide, as described in Bernard et at. 2002.

### Example 4

### Protection from Adverse Conditions

A. Enhancing Hypoxia Tolerance in Mice

Experiments were conducted to test the ability of a mouse in a ‘hibernation-like’ state to survive in conditions where it would normally die. The adverse condition was hypoxia, which the literature states that mice (C57BL6/J males) may typically survive for 20 minutes at 5% oxygen. Zhang et al. 2004.

As shown in Table 2, the experiment involved exposing the mouse to 80 ppm (unless otherwise noted) H₂S for the time indicated, followed by the decrease in oxygen tension in the chamber, while still under H₂S. The hypoxic exposure was timed (indicated below) and viability of the mice was determined.

Short exposures of the mice to H₂S (at least at 80 ppm) were less successful at protecting the mouse from hypoxia, although there was at least one that did survive a 50-minute hypoxic exposure after just 8 minutes in H₂S. Furthermore, it was observed that a mouse exposed to 90 ppm H₂S for just 10 minutes did survive much longer in the 5% oxygen condition, although it did eventually expire.

Exposing the mice to 80 ppm H₂S for longer periods of time had a strong effect on protecting them from hypoxia for up to an hour.

TABLE 2

| | Time in H2S | | | | |
|---|---|---|---|---|---|
| | Prior to | | | | |
| Ambient | hypoxic | | | Time in | |
| Temp | exposure | | Oxygen % | Hypoxia | Result |
| 20° C. | 5 | hrs | 5.20% | 11 minutes | life |
| 20° C. | 5.5 | hrs | 5.00% | 25 minutes | life |
| 20° C. | 5 | hrs | 5.00% | 60 minutes | life |
| 20° C. | 5 | hrs | 4% | 28 minutes | life |
| 24° C. | no H2S | | 5% | 14 minutes | dead |
| 24° C. | simultaneous | | 5.10% | 10 minutes | dead |
| 24° C. | 8 | minutes | 5% | 20 minutes | dead |
| 24° C. | 8 | minutes | 4.00% | 8 minutes | dead |
| 24° C. | 8 | minutes | 4.50% | 23 minutes | dead |
| 30° C. | 8 | minutes | 4.50% | 6 minutes | dead |
| 24° C. | 10 | minutes | 5% | 56 minutes | dead |
| | (90 | ppm) | | | |
| 24° C. | 8 | minutes | 5.00% | 50 minutes | life |

B. Enhancing Anoxia Tolerance in Flies

1. Background

The use of carbon dioxide (CO) and hydrogen sulfide (H₂S) to enhance the survival of a complex metazoan, Drosophila melanogaster, in anoxia was investigated. These experiments indicated that these agents, especially H₂S, can increase the anoxia tolerance of adult D. melanogaster.

C. elegans embryos survive in anoxia (<10 ppm O₂) by entering into suspended animation, and development can proceed in 0.5% O₂. However, there is a 10-fold range (0.01-0.1% O₂) of lethal oxygen concentrations. Moreover, preventing oxygen utilization with carbon monoxide can prevent hypoxic damage in embryos. Thus, if there is not enough oxygen available for efficient biological activity, then it is better to not have (or use) any oxygen.

In more complex metazoans, the cellular oxygen concentration is not necessarily the same as the environmental oxygen levels. In C. elegans, oxygen is delivered to the tissue by diffusion. However, in higher organisms there are proteins that bind oxygen in order to transport it to the tissues, such as hemoglobin. Therefore, when environmental oxygen levels drop, there may be residual oxygen at the cells.

Most organisms are not able to survive exposure to environmental anoxia. One possibility is that the residual oxygen at the cellular level is toxic, corresponding to the lethal oxygen range observed in C. elegans embryos. In this scenario, survival of anoxia would be enhanced if the residual oxygen was removed or made un-utilizable. CO₂ promotes the release of O₂ from hemoglobin and H₂S is a potent inhibitor of oxidative phosphorylation.

2. Materials and Methods

Basic experimental setup. Adult flies were introduced into 35 mL tubes made of glass with a gas-tight rubber stopper (Balsh tubes). This was usually accomplished by anesthetizing flies with CO₂, moving groups of flies to vials with food to recover for at least 2 hours, and then transferring them into the Balsh tube. To exchange the gaseous environment in the Balsh tube, two 18 gauge needles were inserted into the rubber stopper, and gas is blown into one of the needles at 100 mL/min. To prevent dessication, gasses were humidified by bubbling through 10 mL of water before passing it through the Balsch tube. The water in the bubbler is equilibrated with the gas for at least 20 minutes before starting the experiment.

For “stopped-flow” experiments, gas exchange proceeded for 60 minutes before sealing the tube. For “low-flow” experiments, gas flow continued throughout the experiment. CO₂ was from the house source (100%), and anoxic environments were established by flushing out room air with 100% nitrogen (N₂). Care was taken to prevent introduction of room air into the system while switching the atmosphere from CO₂ to N₂.

After anoxic treatment, oxygen was reintroduced into the Balsh tube by flushing with house air for 20 minutes. The rubber stopper was then removed and a food vial is inverted over the top of the Balsh tubes with Parafilm. Flies were scored as alive if they resume movement. Viability was scored at least 18 hours after the end of anoxic treatment. After two weeks, if the food vials contained larvae and/or pupae the flies were considered to be fertile.

3. Results

Treatment with CO₂ prior to anoxic exposure. Adult flies exhibited a higher rate of anoxic survival if they are first pretreated with CO₂. After a 19 h anoxic exposure in a stopped-flow experiment, adult flies pretreated with CO₂ for 30 or 90 minutes exhibited 54% or 28% survival, respectively. No survival was observed in controls exposed to anoxia without CO₂ pretreatment or CO₂ without subsequent anoxic exposure. Furthermore, no flies survived anoxic exposure with CO₂ pretreatment if they were also exposed to CO₂ immediately following anoxic exposure for 20 minutes.

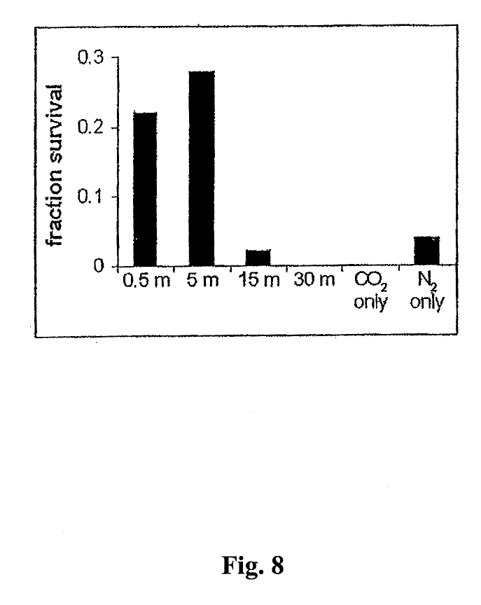

A short exposure to CO₂ was sufficient for enhanced survival of anoxia. In stopped-flow experiments with 22 h anoxic exposure, the fraction of flies that survive was highest if CO₂ was administered for 0.5-5 minutes before switching to the nitrogen atmosphere (FIG. 8). Thus, for subsequent experiments, the standard protocol was to treat with CO₂ for 10 minutes before anoxic exposure. In a low-flow experiment using this protocol, 6% of adult flies survived a 20 h anoxic exposure, and this survival required the CO₂ pretreatment.

Experiments suggested that it is important to prevent reintroduction of O₂ between the CO₂ treatment and establishing the N₂ environment. When the water in the bubbler used to humidify the air was not equilibrated with N₂ before flushing out the CO₂, no flies survived a 13 h anoxic exposure in these experiments, whether the N₂ was introduced at 10, 50, or 100 mL/min. Under these conditions, the CO₂ atmosphere was flushed out with a N₂/O₂ mix resulting from the O₂ dissolved in the water.

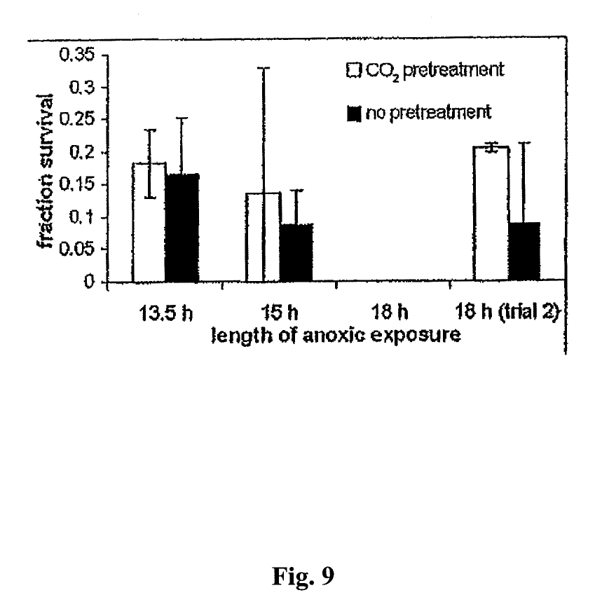

A series of low-flow experiments were conducted to determine the time of anoxic exposure that can be tolerated with CO₂ exposure compared to no pretreatment, testing each condition in duplicate (FIG. 9). In these data, the trend is that CO₂ pretreatment results in greater survival. An important caveat is that these experiments deviated from the standard protocol in that the flies were anesthetized with CO₂ and transferred to the Balsh tubes and allowed to recover for only 10-20 minutes before initiating the CO₂ treatment (except for Trial 2 of the 18 h timepoint).

Several other experiments were performed that were not informative to whether pretreatment with CO₂ was beneficial. For instance, in one experiment no survival was observed after 17, 22, and 24 h of anoxia in a low-flow experiment with a 10 min CO₂ pretreatment period. However, in other experiments, many flies survived after 17 h. This may indicate that in certain cases other factors affect the outcome, such as age of the adults, circadian rythms or variations in room temperature. In another experiment to compare the stopped-flow setup to the low-flow setup, no flies survived a 17 or 19.5 h anoxic exposure; however, in this instance mold contamination may have contributed to the demise of the flies.

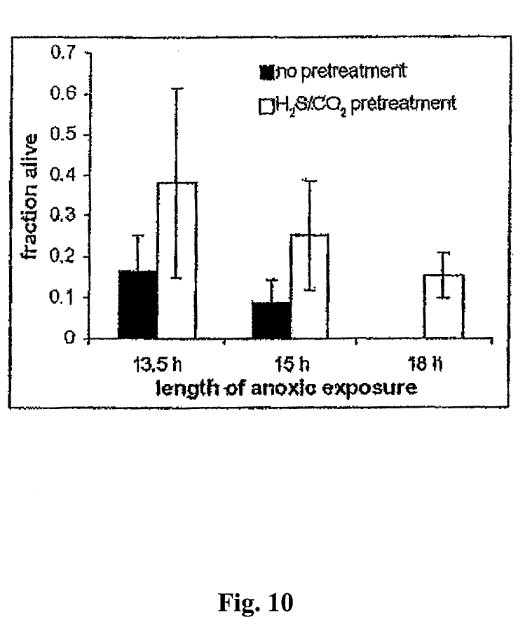

Treatment with H₂S prior to anoxic exposure. Including H₂S in the pretreatment protocol more dramatically enhanced the ability of adult flies to survive anoxia. In a series of experiments analogous to those shown in FIG. 9, adding 50 ppm H₂S to the CO₂ pretreatment (H₂S/CO₂) increased the fraction of flies that survived treatment (FIG. 10). These flies seem healthy, and produced progeny after exposure. However, in a similar experiment no flies survived 1S, 20, 25, or 30 h in anoxia after 10 minutes in H₂S/CO₂. The cause of this discrepancy is unclear. Consistent with a beneficial influence of H₂S treatment, after a 15 h anoxic exposure 50% of flies pretreated with H₂S survived, whereas there was no recovery of control flies that were not exposed to H₂S. In this experiment, the flies were treated with CO₂ for 10 min, then H₂S/CO₂ for 10 min, then N₂/H₂S for 10 min, and finally with N₂ for the duration of the low-flow experiment.

CO₂ treatment is not required for the H₂S-dependent increase in survival of anoxia. 25% of flies treated with 50 ppm H₂S in room air prior to being made anoxic for 18.5 h survived. The fraction of flies surviving was unaffected if a 10 min exposure to H₂S/CO₂ was added before establishing the anoxic environment. In a control experiment where flies were treated only with CO for 10 min before the anoxic exposure, only 11% of the flies recovered.

The time at which H₂S is administered appears important for enhancing anoxic survival. If H₂S is present throughout the anoxic exposure (20 h) no flies recover, whether H₂S was present during the CO₂ pretreatment or not. However, 35% of flies survive if 50 ppm H₂S is present in the CO₂ pretreatment and then is removed as the anoxic environment is established. In parallel experiments, 6% of flies exposed to anoxia after 10 min pretreatment with CO₂ (no H₂S) survived.

Preliminary experiments with larvae and embryos. The enhanced survival of anoxia after treatment with CO and CO with H₂S is also observed in embryos and larvae. After exposure to anoxia for 24 h, 7 pupae were formed from a pool of 0-19 h old embryos. However, 20 pupae were observed from a matched pool that was pretreated with CO₂ for 10 min. Similarly, larvae exposed to 24.5 h anoxia can resume movement upon reoxygenation only if they were pretreated with CO₂ or H₂S/CO₂. 0-24 h old embryos survive 18.5 h anoxic exposure and develop to adulthood whether pretreated with CO₂ or H₂S/CO₂.

Cold treatment during anoxia. Decreasing the environmental temperature may extend the length of time that adult flies can survive anoxic exposure. At room temperature, no flies survived a 15.5 h anoxic exposure in a stopped-flow setup, but 20% of those kept at 4° C. while anoxic recovered. Similarly, no flies survived being transitioned to anoxia at 4° C. and then moved to room temperature for 16.5 h. However, after 16.5 and even 40 h flies that were kept at 4° C. during the entire exposure recovered and were fertile. Pretreating with CO₂ before establishing anoxic environment did not have a noticeable difference in these experiments.

### Example 5

### Animal Studies using an Active Compound and Hypoxic Conditions

The studies shown in Example 4 demonstrated that prior and continuous treatment of male C57BL/6 mice with H₂S can enhance their ability to survive under hypoxic conditions of 5% oxygen or 4% oxygen.

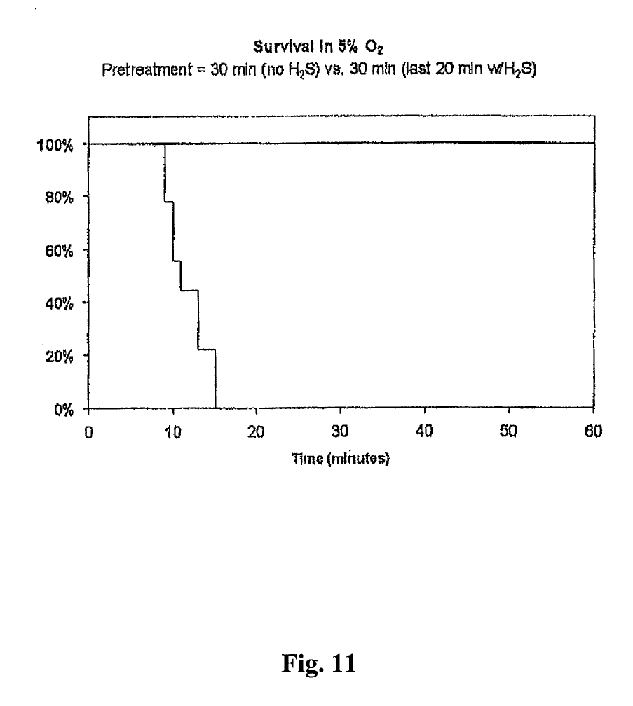

To determine the effect of H₂S pre-treatment alone on survivability under hypoxic conditions (without continuous H₂S exposure during hypoxia), mice were exposed to either 30 minutes of room air (No PT) or 10 minutes of room air followed by 20 minutes of 150 ppm H₂S in room air (PT) before exposure to 5% O₂ (5%), 4% O₂ (4%), 1 hr 5% O₂ followed by 4% O₂ (4%+1 hr 5%), or 1 hr 5% O₂ followed by 3% O₂ (3%+1 hr 5%), and their survival time determined. Experiments were stopped at 60 minutes, and animals still alive were returned to their cage. As shown in FIG. 11, all of the mice in a cohort of animals pre-exposed to 150 ppm H₂S in room air for 20 minutes survived subsequent exposure to 5% O₂, while all of the control animals exposed to room air alone had died within 15 minutes of exposure to 5% O₂. Thus, pre-exposure of mice to H₂S establishes a physiological state in the mice that allows prolonged survival to otherwise lethal hypoxia. The protection observed in H₂S pre-treated mice far exceeds the known protective effect of whole body hypoxia preconditioning that has been reported in the literature, in which survivability in 5% O₂ was extended only twofold (Zhang et al. 2004). Although not shown in FIG. 11, some H₂S pre-treated mice were able to survive for more than four hours in 5% O₂ and were able to recover with no noticeable motor or behavioral deficits.

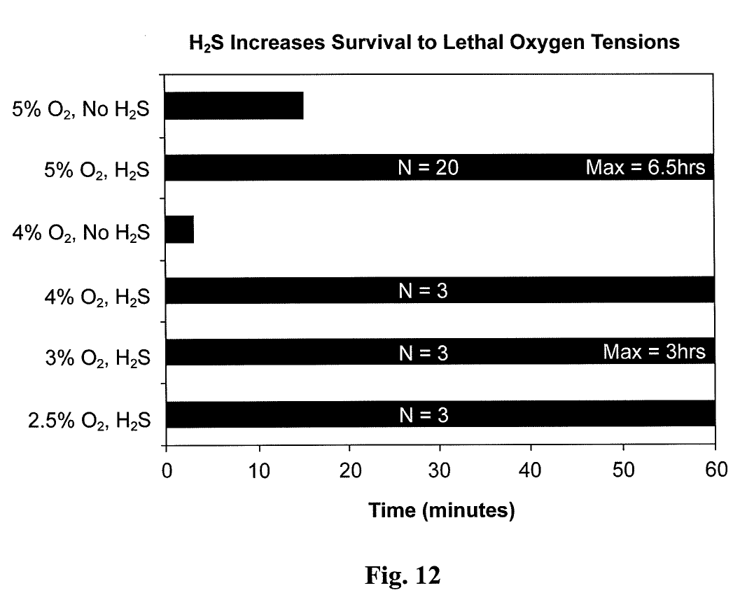

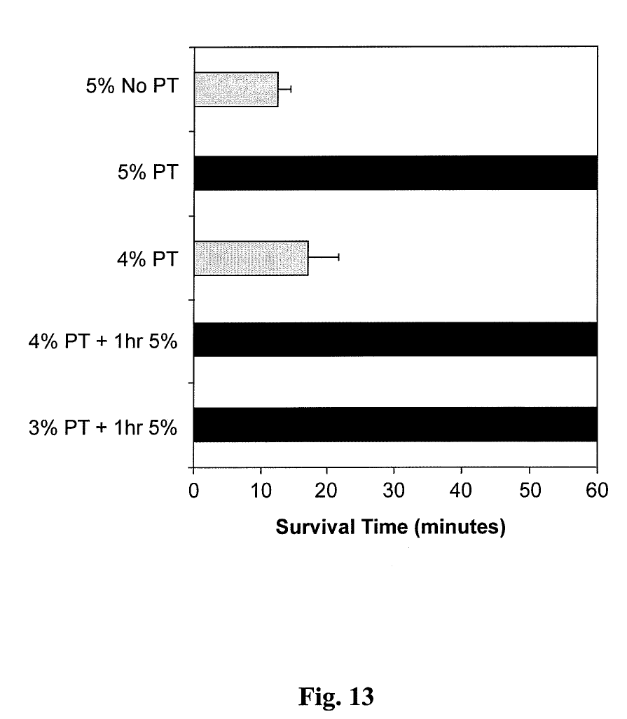

To determine if H₂S pre-treatment enhances survivability to even lower oxygen tensions, mice were exposed to lower O₂ concentrations. As shown in FIG. 12, H₂S pre-treatment greatly enhances survival in the presence of 5% O₂. In contrast, H₂S pre-treatment provided a small increase in survival in the presence of 4% O₂. However, if H₂S pre-treated mice were exposed to a step-wise reduction in O₂ levels, such that they were first pre-treated and then exposed for 1 hour to 5% O₂ and then exposed to either 4% O₂ or 3% O₂, their survival time was enhanced to the same level as that observed when they were exposed to 5% O₂ following H₂S pre-treatment (FIG. 13). Thus, pre-exposure to H₂S establishes a physiological state in which mice can survive a graded reduction in oxygen tensions exceeding 80% (21% normoxia reduced to 3% O₂). Furthermore, in some experiments, graded reduction of oxygen tension following H₂S pre-treatment showed the mice can survive for an hour in oxygen tensions as low as 2.5%.

These data and those described in Example 4 demonstrate that exposure to H₂S has a pharmacological effect in which survival in otherwise lethal hypoxia is greatly enhanced. In this context, the pharmacological effects of 128 depend on dose levels and duration of exposure to H₂S, parameters that one skilled in the art can vary to achieve optimum survivability to lethal hypoxia. One skilled in the art will appreciate that the route of administration (e.g., inhaled versus parenteral administration) can also be varied to achieve the desired effect of lethal hypoxia tolerance in a mammal. In addition, the pharmacological effect can be observed either when H₂S exposure is limited to pre-treatment or is extended into the period of hypoxia. Likewise, the timing of exposure to H₂S relative to the onset of lethal hypoxia can be varied to maximize the enhanced survivability. These data are consistent with the hypothesis that reduction in oxygen demand resulting from pretreatment with an active compound, such as an oxygen antagonist, allows survival in reduced oxygen supply that is otherwise lethal to the animal.

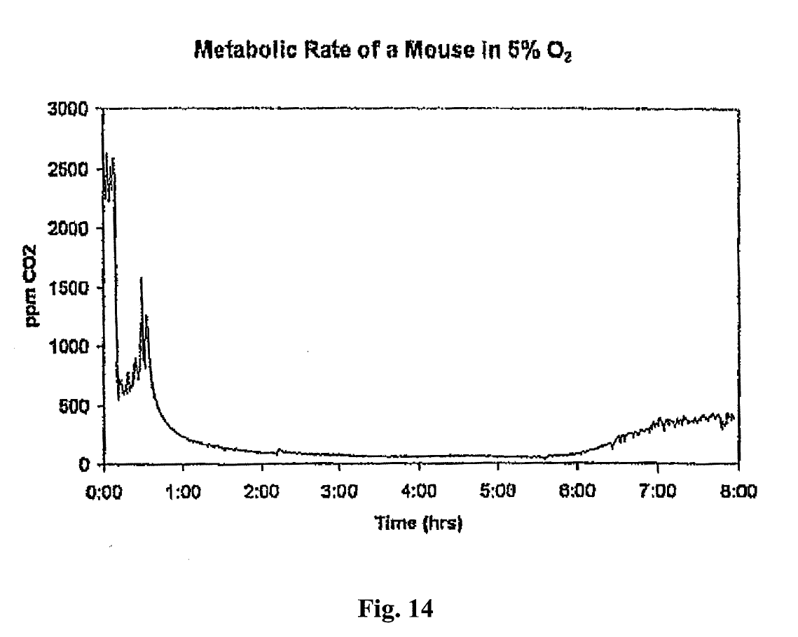

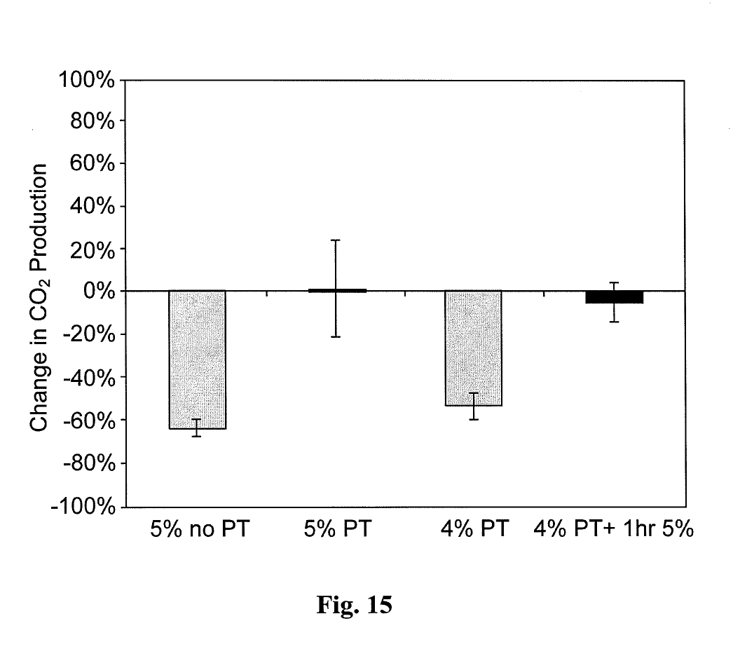

To characterize the changes in metabolism that occur in the setting of enhanced survivability to lethal hypoxia afforded by H₂S treatment, CO₂ production by the mice was measured during exposure to H₂S and thereafter following termination of H₂S treatment and subsequent exposure to 5% O₂. The change in CO₂ production is shown in FIG. 14. Changes in CO₂ production upon transition to either 5% O₂ or 4% O₂ were measured in mice exposed to either room air for 30 minutes (No PT) or room air for 10 minutes followed by 150 ppm H₂S for 20 minutes (PT). In addition, the change in CO₂ production upon step-wise transition to 5% O₂ for 1 hr followed by 4% O₂ was measured. The results of these experiments are provided in FIG. 15.

CO₂ production was reduced approximately two to three-fold in the first five to ten minutes of H₂S pre-treatment, suggesting that stasis is induced in the mice during the minute pre-treatment with 150 ppm H₂S in room air. However, O₂ consumption and core body temperature of the animals did not change significantly during the H₂S pre-treatment, suggesting that a physiological state other than stasis may be established in the mice during exposure to H₂S that allows enhanced survivability to lethal hypoxia. Such a state might be characterized by a reduction in metabolism within the biological material of a magnitude that is less than that defined as stasis. In order to achieve stasis using an active compound, the biological matter necessarily must transition through a graded hypometabolic state in which oxygen consumption and CO₂ production are reduced less than twofold in the biological matter. Such a continuum, in which metabolism or cellular respiration is reduced by an active compound to a degree less than twofold, is described as a state of “pre-stasis.” Continued monitoring of CO₂ production following termination of H₂S pre-treatment and induction of lethal hypoxia shown in FIG. 14 demonstrates an approximately 50-fold reduction in CO₂ production, indicating that stasis is achieved during the exposure to lethal hypoxia. A concomitant decrease in O₂ consumption and strong attenuation of motility in the mice during exposure to lethal hypoxia further supports the observation that stasis is subsequently achieved during exposure to lethal hypoxia.

Changes in CO₂ production associated with transition to hypoxic conditions of either 5% O₂ or 4% O₂ after H₂S pre-treatment or no pre-treatment were measured. As shown in FIG. 13, mice exposed to either 5% O₂ in the absence of H₂S pre-treatment or exposed to 4% O₂ in the presence of H₂S pretreatment displayed a substantial decrease in CO₂ production. In contrast, H₂S pre-treated mice that were subsequently exposed to either 5% O₂ or 5% O₂ followed by 4% O₂ did not show any significant changes in CO₂ production as compared to the new baseline level following H₂S pre-treatment. These results demonstrate a correlation between reduced metabolic activity and death associated with exposure to 5% O₂ in the absence of H₂S pre-treatment or exposure to 4% O₂ with H₂S pre-treatment. In addition, these data demonstrate that exposure to 5% O₂ or a step-wise reduction from 5% O₂ to 4% O₂ following H₂S pre-treatment does not result in an additional reduction in metabolic activity. To summarize these results, decreases in CO₂ evolution that occur upon transition form normoxia to lethal hypoxia were blunted in mice that were pre-treated with H₂S. Transition from normoxia to lethal hypoxia caused a 40% reduction in CO₂ evolution, but pre-treatment with H₂S, while itself causing a 50-60% reduction in CO₂ evolution to a new, lower baseline, prevented any further decrease in CO₂ evolution on transition to lethal hypoxia. These data demonstrate that H₂S pretreatment alone prevents additional reductions in metabolic activity typically associated with a transition to lethal hypoxia, thereby enhancing survival under hypoxic conditions. In addition, these data support a model wherein pre-exposure of biological matter to active compounds is sufficient to enhance survivability and/or reduce damage from injuries or disease insults.

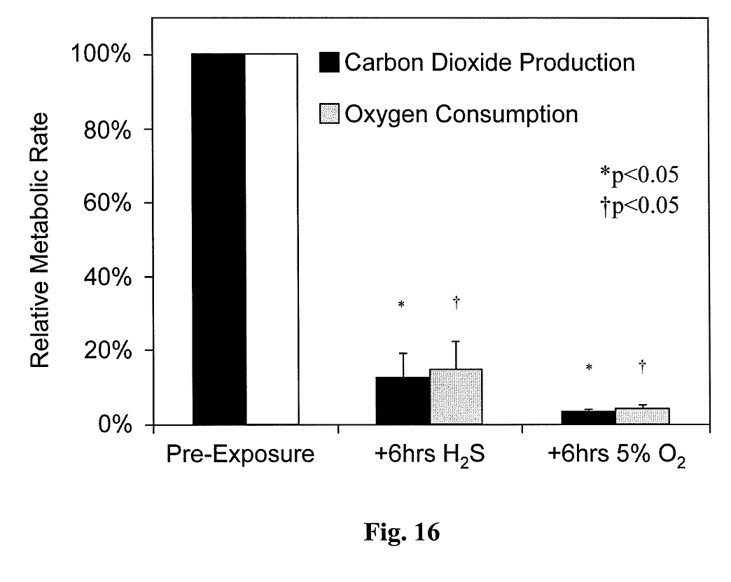

The longest that a pretreated mouse was exposed to 5% oxygen was for 6.5 hours and the mouse survived with no apparent harmful effects. This was the longest exposure tested and demonstrates a significant difference between treated and untreated mice. During this long exposure to 5% oxygen, the metabolic rate of the mouse dropped by over 50-fold, compared to a 10-fold drop using H₂S alone for the same period of time FIG. 16) as we have previously reported (Blackstone et al., 2005). These metabolic rate experiments were done at Tₐ=13° C. so they could be directly compared to our previous studies. At Tₐ=23° C. there is a similar drop in metabolic rate.

2. Discussion

There appears to be a fundamental difference in the pretreated vs. untreated mice. A pretreatment of 150 ppm H₂S for only 20 minutes is long enough to increase the time of survival dramatically. These pretreated mice can survive for over six hours in 5% O₂, whereas untreated mice survive for less than 20 minutes at this O₂ concentration. Additionally, we have devised a pretreatment protocol (see results) to enable mice to survive for several hours at only 3% O₂. These pretreatment protocols reduce the metabolic rate of the mice prior to the exposure to lethal O₂ tensions. The drop in metabolic rate we see with the pretreatment is similar to that seen in hibernating animals (Heldmaier et al. 2004) and may represent a state that is closer to hibernation than seen previously (Blackstone et al. 2005).

It is thought that the supply and demand of cellular energy must be in balance for organismal survival (Hochachka and Lutz, 2001). Gradient organisms position themselves at optimal points along an oxygen/sulfide gradient (Fenchel et at. 1995) which may be how they maintain energetic balance. Perhaps there is a similar energetic system working within the cells of larger organisms. While the energetics of the supply side of metabolism have been intensively studied, the demand side has been largely ignored (Suarez and Darveau, 2005)—possibly due to the apparent complexity of energy using processes within the cell. Interestingly, the process that utilizes oxygen, cytochrome c oxidase (COX), is universally lumped with supply processes (for one example see Bishop et al. 2002). Perhaps this is a fallacy and instead COX could be thought of as the place where supply and demand meet in aerobic cells. Making the process that consumes oxygen part of demand may help elucidate processes involved with metabolic control.

H₂S is a reversible inhibitor of COX (reviewed in Beauchamp et al. 1984), is made endogenously by mammalian cells (reviewed in Kimura et al 2005), and can put a mammal into a suspended animation-like state that is entirely reversible when administered at low levels (Blackstone et al. 2005). The low levels of this COX inhibitor may be lowering the oxygen demand for the animal, thus shifting the metabolic curve to be less dependent upon supply. This effect could be the result of a shift in the redox state discussed above, or could be independent of that effect depending on the sensitivity of the cell to redox state versus the sensitivity of COX to this inhibitor. Putting COX on the demand side of the equation is supported by the idea that stressful situations such as low oxygen supply create large amounts of reactive oxygen species (RDOS) that may cause the actual damage seen in these ischemic cells/tissue/organisms (for review on ROS see Becker 2004). In these cases, O₂ supply is vastly reduced without a check on demand, and the inefficiencies of the electron transport system in these situations are thought to create ROS. If, however, demand is suppressed prior to the reduction of supply, then ROS should not be generated and damage to the cell would be avoided. The results shown above are consistent with the idea that COX is part of metabolic demand. In future studies we hope to measure the change in sulfide concentration in a variety of tissues to elucidate the important processes for protection that may be occurring at the cellular level.

In summary, then, on-demand induction of a suspended animation-like state in mice, using H₂S, dramatically increases survival times in lethal hypoxia, possibly by buffering oxygen consumption, altering the redox environment within cells, and preventing a lethal imbalance between energy supply and demand.

### Example 6

### Adaptation to H₂S Increases Thermotolerance and Lifespan in C. elegans

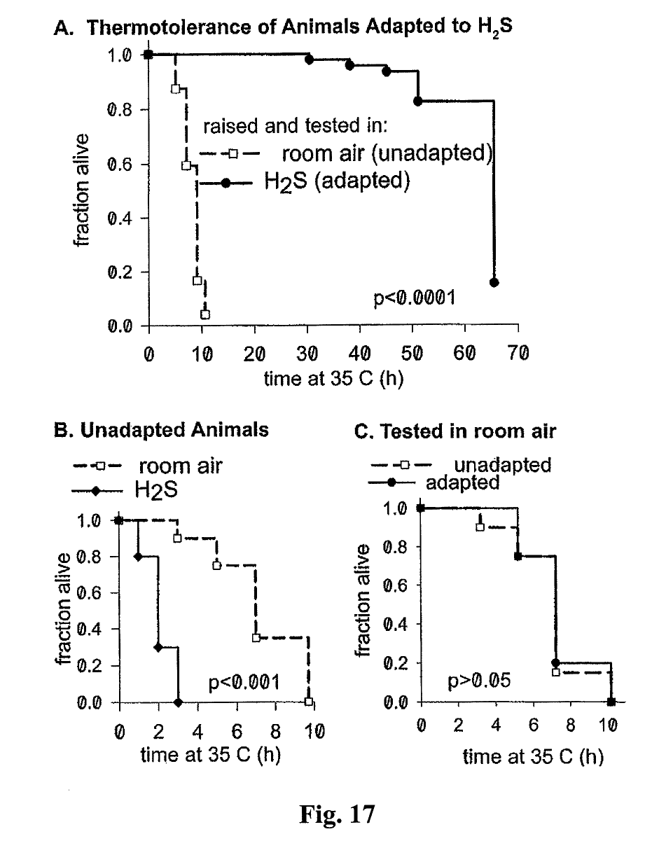

We hypothesized that the physiological alterations of adaptation to H₂S may have other benefits. Adaptation to H₂S results in increased thermotolerance in C. elegans (FIG. 17). At high temperature, adapted animals typically have a mean survival time up to 8 times longer than unadapted controls. Although the maximum extension of survival time observed varied between experiments, the effect was quite robust with an average of 80% of H₂S-treated animals alive when all untreated animals had died (data not shown). Unadapted animals were generally more sensitive to thermal stress in the presence of H₂S, demonstrating that H₂S does not act directly to prevent damage associated with thermal stress (FIG. 17B). Moreover, unlike thermotolerance induced by prior stress such as heat shock or azide, continuous exposure to H₂S is required for thermotolerance of adapted animals (FIG. 17C). These data show that adaptation to H₂S enables thermotolerance, and suggest that this phenotype can be distinguished from tolerance to high concentrations of H₂S.

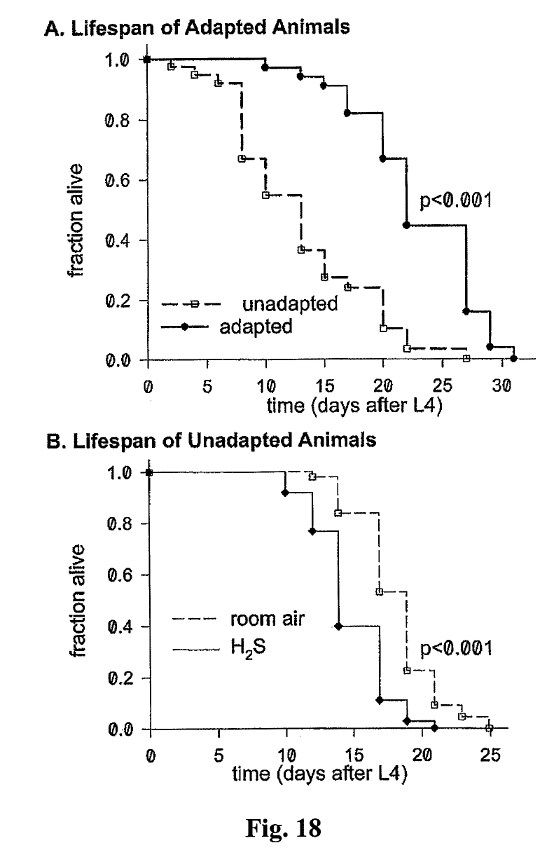

In C. elegans, resistance to high temperature is often correlated with increased lifespan. Indeed, we observed that animals adapted to H₂S were long-lived compared to controls (FIG. 18). The mean lifespan of adapted animals increased by 70% relative to the unadapted population, and maximum lifespan was also lengthened. We have also observed that the life expectancy increase appears to be an overall increase, as well as an increase in “adult” life expectancy (ost-sexual maturity). Increased lifespan is not observed when animals are moved to the H₂S-containing atmosphere at the beginning of the lifespan experiment (as L4 larvae), indicating that this effect requires prior adaptation to H₂S (FIG. 18B). In fact, the lifespan of these animals is slightly shorter than untreated controls. We conclude that the increase in lifespan is a feature of the physiological alterations resulting from the adaptation to H₂S.

Increased thermotolerance and lifespan of adapted animals does not appear to be associated with reduced metabolic activity. Animals raised in low H₂S are visually indistinguishable from untreated controls, and produce similar numbers of progeny (221±35 in H₂S compared to 234±15 in control conditions). Neither embryonic nor post-embryonic development is delayed in adapted animals (Table 1). In addition, the rate of egg-laying is not noticeably changed by H₂S (Table 2). The rate of egg-laying is tightly correlated with oocyte production, an energetically expensive activity that is a sensitive readout of metabolic capacity. Consistent with this, we observe a two-fold decrease in the rate of egg-laying when ambient O₂ tension is reduced to 2% (from 21% O₂ in air), a perturbation that decreases the metabolic rate of worms by ˜30%. H₂S does not further alter the rate of egg-laying in environments with reduced ambient O₂ (Table 2). We also have observed that animals exposed early to H₂S reach sexual maturity at the same time with no delays as wild-types, and are otherwise normal. Although we cannot definitively conclude that mitochondrial energy production has not been affected in our conditions, these data demonstrate that overall metabolic output is not appreciably decreased.

TABLE 1
Developmental Rates

| | embryogenesis^{a} | post-embryonic^{b} |
|---|---|---|
| | hours ± SEM (n) | hours ± SEM (n) |
| unadapted^{c} | 12.6 ± 0.4 (43) | 49.6 ± 0.2 (15) |
| adapted^{d} | 13.3 ± 0.4 (34) | 49.3 ± 0.2 (15) |
| clk-1(qm30)^{e} | 16.9 ± 2.0 (17)* | 69.0 ± 0.3 (15)* |

^{a}Median time for 2-cell embryos to hatch at room temperature from one representative experiment.
^{b}Median time for starved first-stage larvae to become gravid adults after return to food in one representative experiment.
^{c}Wild-type animals in room air.
^{d}Wild-type animals in 50 ppm H₂S.
^{e}This mutant has been reported to develop slowly (wong ref).
*significantly different from wild-type, unadapted controls p < 0.05.

TABLE 2
Rate of Egg-Laying in H₂S

| | room air^{a} | H₂S | 2% O₂ | 2% O₂ + H₂S |
|---|---|---|---|---|
| unadapted^{b} | 7.8 ± 1.5 | 8.3 ± 3.0 | 4.2 ± 1.3 | 4.5 ± 1.6 |
| adapted^{c} | nd | 7.8 ± 2.0 | 4.9 ± 1.4 | 4.7 ± 1.2 |
| clk-1(qm30) | 4.2 ± 0.6* | 3.2 ± 1.1* | 2.4 ± 0.8* | 2.5 ± 1.0* |

^{a}Average rate of egg-laying in one representative experiment in embryos per hour ± SD. n = 10 individuals. nd; not done
^{b}Wild-type animals grown in room air.
^{c}Wild-type animals in 50 ppm H₂S.
*signficanly different from wild-type unadapted controls in the same column (p < 0.05 by two-tailed t-test).

Several genetically-independent pathways that can increase lifespan have been characterized in C. elegans. We reasoned that the adaptation to H₂S may result in the increase in thermotolerance and lifespan by influencing one of these pathways. To evaluate this possibility, we tested whether mutants defective in each pathway exhibited increased thermotolerance after adaptation to H₂S.

In C. elegans the insulin/IGF signaling (IIS) pathway regulates the decision to enter into an alternative larval stage, the dauer, upon exposure to unfavorable conditions such as high population density, low food or high temperature. Mutations in the insulin-like receptor DAF-2 that reduce IIS increase the probability of entry into the dauer state, and in adults increase thermotolerance and lengthen lifespan even without entry into dauer. DAF-16 is required for all phenotypes of daf-2. Our data suggest that adaptation to H₂S does not result in decreased IIS. First, H₂S exposure starting in adulthood does not increase lifespan (FIG. 17B), whereas RNAi of daf-2 starting in adulthood is sufficient to increase lifespan. Second, adaptation to H₂S occurs in daf-16(m26) mutants and the daf-2(e1370) mutant is even more resistant to thermal stress after adaptation to H₂S (data not shown). Finally, H₂S does not induce entry into the dauer state in wild-type worms, as post-embryonic development time is not extended, nor does it prevent entry into or exit from dauer in daf-2(e1370) mutants. These data suggest that the mechanism by which H₂S increases lifespan and thermotolerance are independent of the IIS pathway.

Reduction of mitochondrial function is a well-established mechanism for increasing lifespan of C. elegans. In vitro, H₂S is an inhibitor of cytochrome c oxidase, the terminal enzyme in the electron transport chain. Thus, an attractive hypothesis is that the adaptation to H₂S acts to extend lifespan and thermotolerance by reducing mitochondrial activity. Decreasing mitochondrial function by RNAi during adulthood does not increase lifespan, just as addition of H₂S to adults does not increase lifespan (FIG. 18B). However, adapted animals do not share hypometabolic phenotypes commonly observed in C. elegans with defective mitochondrial function, including long-lived isp-1 and clk-1 mutants (Table 1). We observed that long-lived mutants isp-1(qm150) and clk-1(qm30) that are defective in mitochondrial function adapted to H₂S and became thermotolerant (data not shown), suggesting that the mechanisms required for adaptation to H₂S are genetically distinct from mitochondrial dysfunction.

Dietary restriction (DR) extends lifespan in a wide range of organisms. C. elegans that have reduced rates of pharyngeal pumping are long-lived, likely as a result of DR. These animals appear thin and pale, develop slowly and have reduced fecundity, which are phenotypes not observed in animals adapted to H₂S. Therefore, we considered it unlikely that H₂S acts through the DR pathway. Consistent with this interpretation, eat-2(ad1116) mutant animals that are long-lived due to DR become thermotolerant upon adaptation to H₂S (data not shown). We conclude that the adaptation to H₂S alters the physiology of worms in a manner distinct from DR, suggesting that it acts through a separate mechanism.

In addition to, but perhaps overlapping with, these genetically-defined pathways, sirtuins influence lifespan in many organisms. Therefore, we investigated the role of sirtuin function in adaptation to H₂S. We observed that thermotolerance and lifespan of sir-2.1(ok434) animals is not increased by adaptation to H₂S (FIG. 19). These data indicate that SIR-2.1 activity is required for increased thermotolerance and lifespan upon adaptation to H₂S. However, SIR-2.1 is not required for all phenotypes associated with the adaptation to H₂S. sir-2.1(ok434) animals grown in low concentrations of H₂S all survive high concentrations of H₂S similar to wild-type (data not shown). We conclude that SIR-2.1 activity is required for a subset of phenotypes associated with adaptation to H₂S. In addition, the finding that SIR-2.1 activity is required for increased thermotolerance and lifespan in H₂S further suggests that these phenotypes are not due to non-specific metabolic suppression.

In summary, this data demonstrates that C. elegans adapt to H₂S and this adaptation results in physiological alterations that are manifested as increased resistance to thermal stress, increased lifespan and tolerance to otherwise lethal H₂S concentrations. Thus, H₂S expands the range of conditions in which C. elegans can survive. Perhaps endogenous H₂S naturally regulates SIR-2.1 activity to coordinate response to environmental changes. Mice exposed to H₂S also show dramatic changes in physiology that protects them against otherwise lethal hypoxia. These results suggest that H₂S might be a useful therapeutic agent for many pathological states. Defining how organisms adapt to H₂S may yield insights into similar mechanisms in higher organisms, including humans, with potentially wide-ranging implications in both basic research and clinical practice.

### REFERENCES

The following references, to the extent that they provide exemplary procedural or other details supplementary to those set forth herein, are specifically incorporated herein by reference.
U.S. Pat. No. 3,777,507
U.S. Pat. No. 3,881,990
U.S. Pat. No. 3,989,816
U.S. Pat. No. 3,995,444
U.S. Pat. No. 4,034,753
U.S. Pat. No. 4,186,565
U.S. Pat. No. 4,266,573
U.S. Pat. No. 4,292,817
U.S. Pat. No. 4,354,488
U.S. Pat. No. 4,442,856
U.S. Pat. No. 4,444,762
U.S. Pat. No. 4,447,415
U.S. Pat. No. 4,473,637
U.S. Pat. No. 4,502,295
U.S. Pat. No. 4,559,258
U.S. Pat. No. 4,723,974
U.S. Pat. No. 4,745,759
U.S. Pat. No. 4,798,824
U.S. Pat. No. 4,828,976
U.S. Pat. No. 4,938,961
U.S. Pat. No. 4,951,482
U.S. Pat. No. 5,066,578
U.S. Pat. No. 5,157,930
U.S. Pat. No. 5,217,860
U.S. Pat. No. 5,231,025
U.S. Pat. No. 5,285,657
U.S. Pat. No. 5,326,706
U.S. Pat. No. 5,370,989
U.S. Pat. No. 5,395,314
U.S. Pat. No. 5,399,363
U.S. Pat. No. 5,405,742
U.S. Pat. No. 5,434,045
U.S. Pat. No. 5,466,468
U.S. Pat. No. 5,470,738
U.S. Pat. No. 5,474,060
U.S. Pat. No. 5,476,763
U.S. Pat. No. 5,543,158
U.S. Pat. No. 5,552,267
U.S. Pat. No. 5,560,353
U.S. Pat. No. 5,568,910
U.S. Pat. No. 5,569,579
U.S. Pat. No. 5,580,781
U.S. Pat. No. 5,599,659
U.S. Pat. No. 5,636,643
U.S. Pat. No. 5,641,515
U.S. Pat. No. 5,645,081
U.S. Pat. No. 5,692,497
U.S. Pat. No. 5,693,462
U.S. Pat. No. 5,699,793
U.S. Pat. No. 5,719,174
U.S. Pat. No. 5,736,397
U.S. Pat. No. 5,739,169
U.S. Pat. No. 5,752,929
U.S. Pat. No. 5,801,005
U.S. Pat. No. 5,830,880
U.S. Pat. No. 5,846,945
U.S. Pat. No. 5,887,611
U.S. Pat. No. 5,912,019
U.S. Pat. No. 5,952,168
U.S. Pat. No. 6,013,256
U.S. Pat. No. 6,046,046
U.S. Pat. No. 6,054,261
U.S. Pat. No. 6,057,148
U.S. Pat. No. 6,100,082
U.S. Pat. No. 6,187,529
U.S. Pat. No. 6,365,338
U.S. Pat. No. 6,490,880
U.S. Pat. No. 6,492,103
U.S. Pat. No. 6,524,785
U.S. Pat. No. 6,552,083
U.S. Pat. No. 6,560,353
U.S. Pat. No. 6,587,443
U.S. Pat. No. 6,602,277
U.S. Pat. No. 6,790,603
U.S. patent application Ser. No. 10/971,575
U.S. patent application Ser. No. 10/971,576
U.S. patent application Ser. No. 10/972,063
U.S. patent Appln. 2005/0217667
U.S. Prov. Appln. 60/513,458
U.S. Prov. Appln. 60/548,150
U.S. Prov. Appln. 60/557,942
U.S. Prov. Appln. 60/827,337
Alam, Antioxid Redox Signal, 4(4):559-62, 2002.
Amersi et al., Hepatology, 35(4):815-823, 2002.
Austin-Ward and Villaseca, Revista Medica de Chile, 126(7):838-845, 1998.
Barton & Ollis, Oxford, UK, Jones (Ed.), Pergamon Press, 3:373-487, 1979.
Baskin and Wang, Tetrahedron Lett., 43:8479-8483, 2002.
Baskin et al., Org. Lett., 4:4423, 2002.
Beauchamp et al., Crit. Rev. Toxicol. 13, 25, 1984.
Beck et al., Proc. Soc. Exp. Biol. Med. 86, 823, 1954.
Becker, Cardiovasc. Res. 61:461-470, 2004.
Behringer et al., Crit. Care Med., 31(5):1523-1531, 2003.
Bellamy et al., Crit. Care Med., 24(2 Suppl):S24-47, 1996.
Bernard et al., J. Thorac. Cardiovasc. Surg. 90:235-242, 1985.
Bernard et al., N. Engl. J. Med., 346(8):557-563, 2002.
Blackstone et al., Science, 308:518, 2005.
Boyce and Ham, J. Invest. Dermatol., 81:335-405, 1983.
Boyce and Ham, J. Tissue Culture Methods, 9:83-93, 1985.
Briese, Neurosci. Biobehav. Rev., 22(3):427-436, 1998.
Brizel, Seminars Radiation Oncol., 8(4Suppl):17-20, 1998.
Brouard et al., J. Biol. Chem., 277(20):17950-17961, 2002.
Bukowski et al., Clinical Cancer Res., 4(10):2337-2347, 1998.
Burns & Murphey, Arch. Biochem. Biophys., 339:33-39, 1997.
Buns et al., Arch. Biochem. Biophys, 10:60-68, 1995.
Cairns et al., J. Am. Chem. Soc., 74:3982, 1952.
Chapter IV; Chapter VI; Chapter VII; Chapter VIII; Chapter IX of Klayman, D. L.; Gunther, W. H. H. Eds, Wiley Interscience, New York, 1973.
Chasteen and Bentley, Chem. Rev., 103(1):1-25, 2003.
Christodoulides et al., Microbiology, 144Pt 11):3027-3037, 1998.
CIIT (Chemical Industry Institute of Toxicology), In: 90 day vapor inhalation toxicity study of hydrogen sulfide, Toxigenics, 420-710, 1983.
Clive et al., J. Org. Chem., 47:1641, 1982.
Cloarec & Charette, Org. Lett., 6:4731, 2004.
Cohen et al., Ann. Thorac. Surg., 67(5):1489-1491, 1999.
Curran, Seminars Radiation Oncol., 8(4Suppl):2-4, 1998.
Davidson et al., J. Immunother., 21(5):389-398, 1998.
Davis (1994)
Demuynck and Vialle, Bulletin de la Societe Chimique de France, 4:1213-1218, 1967
Demuynck et al., Bulletin de la Societe Chimique de France, 3366-3367, 1966.
Demuynck et al., Bulletin de la Societe Chimique de France, 8:2748-2754, 1967.
Dhanasekaran et al., J. Biol. Chem., 279:37575-37587, 2004.
Dillman, Cancer Biother. Radiopharm., 14(1):5-10, 1999.
Dittmer and Hoey, In: The Chemistry of Sulphinic Acids, Esters, and Their Derivatives, Wiley: Chichester, U.K., 239-273, 1990.
Dorman et al Neurotoxicol. Teratol., 22(1):71-84, 2000.
Dulak et al., Antioxid. Redox Signal, 4(2):229-240, 2002.
Duus, In Comprehensive Organic Chemistry: The Synthesis and Reactions of Organic Compounds, 1^{st} Ed., 1994.
Eto et al., Biochem. Biphys. Res. Commun., 293:1483-1488, 2002.
Ganther, Carcinogenesis 20(9):1657-66 (1999)
Gilbert et al., LANCET, 355:375-376, 2000.
Gladysz et al., J. Org. Chem., 43:1204, 1987.
Glass, Phosph. Sulfur Silicon Rel. Elem., 136, 137, 138:159-174, 1998.
Gorman et al., Toxicology, 187(1):25-38, 2003.
Guarente, Mech. Ageing Devet 126:923, 2005.
Guillemin et al., Cell, 89(1):9-12, 1997.
Hanibuchi et al., Intl. J Cancer, 78(4):480-45, 1998.
Hannan et al., JAMA, 290(6):773-780, 2003.
Harris, J. Org. Chem., 25:225, 1960.
Harris, J. Org. Chem., 30:2190, 1965.
Hays, In: Studies of the Effects of Atmospheric Hydrogen Sulfide in Animals, thesis dissertation, University of Missouri-Columbia, 1972.
Heldmaier et al., Respir. Physiol B. Biochem. Mol. Biol. 130:435-459, 2001.
Hellstrand et al., Acta Oncologica, 37(4):347-353, 1998.
Higuchi and Fukamachi, Folia Pharmacologica Japonica, 73(3):307-319, 1977.
Hobert et al., Organometallics, 20:1370, 2001.
Hochachka et al., Comp. Biochem. Physiol B Biochem. Mol. Biol., 130(4):435-459, 2001.
Hochachka et al., Proc. Natl. Acad. Sci. USA, 93(18):9493-9498, 1996.
Hui and Hashimoto, Infection Immun., 66(11):5329-5336, 1998.
Hwang & Greenberg, Biochemistry, 38:14248, 1999.
Hyspler et al., J. Chromatography, 770:255-259, 2002.
Innicenti et al., Bioorg. Med. Chem. Lett. 14, 5769 (2004).
Jiang et al., Am. J. Physiol. Cell Physiol., 280:1140-1150, 2001.
Ju et al., J. Neuropathol. Exp. Neurol., 59(3):241-50, 2000.
Kamoun, Amino Acids 26, 243, 2004.
Kelso et al., J. Biol. Chem., 276:4588-4596, 2001.
Khan et al., Toxicol. Applied Pharmacol., 103:482-490, 1990.
Kilburn and Warshaw, Toxicology Indust. Health, 11(2):185-197, 1995.
Kilburn, Environ. Health, 54(3):150, 1999 Kilburn, Environ. Res., 81(2):92-99,1999.
Knapp and Darout, Org. Lett., 7:203, 2005.
Kontou et al., J Agricultureal and Food Chem., 52:1212, 2004.
Kuroda et al., Transplantation, 46(3):457-460, 1988,
Kuroda et al., Transplantation, 46(3):457-460, 1988.
Lai et al., Biochemistry, 40:4904-4910, 2001.
Langer et al., Biochemistry 33:14034, 1994.
Langer et al., Biochemistry, 33:10867, 1997.
Ledingham et al., Circulation, 82(2):IV351-358, 1990.
Ledingham et al., J. Thorac. Cardiobasc. Surg., 93:240-246, 1987.
Lee et al., J. Nuc. Med. 26:72, 1985.
Liu et al., J. Org. Chem., 67:9267, 2002.
Longo and Kennedy, Cell, 126:257, 2006.
Lundgren-Eriksson et al., Anticancer Res. 2001 September-October; 21(5):3269-74
Mehlhorn et al., Cardiovasc Surg., 9(5):482-486, 2001.
Menasche et al., Eur. J. Cardio. Thorax. Surg., 8:207-213, 1994.
Michaels et al., Circulation, 106(23):e187-190, 2002.
Monaghan and Garai, Practical Ther. 20:24-26 (1924).
Mugesh et al., Chem. Rev., 101:2125, 2001.
Murai and Kato, In: Organoselenium Chemistry: Modern Developments in Organic Synthesis, Wirth (Ed.), Springer, N.Y., Vol. 28, 2000.
Murai, et al., J. Org. Chem., 66:8101, 2001.
Netherton & Fu, Org. Lett., 3:4295, 2001.
Noguchi et al., Biochemistry, 42:11642, 2003.
Nogueira et al., Chem. Rev., 104:6255, 2004.
Nystul et al., Science, 302(5647):1038-1041, 2003.
O'Sullivan et al., J. Am. Chem. Soc., 126:2194, 2004.
Olojo et al., J. Phys. Chem. A, 108:1018, 2004.
Otterbein et al., Am. J. Physiol. Lung Cell Mol. Physiol, 279(6):L1029-L1037, 2000.
Otterbein et al., Trends Immunol., 24(8):449-455, 2003.
Padilla et al., Molec. Biology of the Cell, 13:1473-1483, 2002.
Padilla et al., Proc. Natl. Acad. Sci. USA, 98(13):7331-7335, 2001.
Partlo et al., Neurotoxicology, 22(2):177-189, 2001.
PCT Appln. WO 94/17178
Petersen, Biochemica et Biophysica Acta, 460:299-307, 1977.
Pietras et al., Oncogene, 17(17):2235-2249, 1998.
Punch et al., 2001
Qin et al., Proc. Natl. Acad. Sci. USA, 95(24):14411-14416, 1998.
Quirante et al., J. Org. Chem., 67:2323, 2002.
Rager et al., NC Med. J., 65(1):18-25, 2004.
Reigan et al. J. Med. Chem., 48:392, 2005.
Remington's Pharmaceutical Sciences, 15^{th} ed., pages 1035-1038 and 1570-1580, Mack Publishing Company, Easton, Pa., 1980.
Rogers et al., Mamm. Genome, 8:711-713, 1997.
Ryter and Otterbein, BioEssays, 26:270-280, 2004.
Seburg abd Squires, Intl. J. Mass Spectrometry Ion Proc., 167/168:541, 1997.
Semenza, Cell, 98(3):281-284, 1999.
Semenza, Trends Mol. Med., 7(8):345-350, 2001.
Shaw (1996)
Shawali et al., J. Org. Chem., 61:4055, 2001.
Shen et al., J. Agric. Food Chem., 50:2644, 2002.
Smith et al., Eur. J. Biochem., 263:709-716, 1999.
Soledad et al., Org. Lett., 3:1213, 2001.
Steudel, Chem. Rev., 102:3905, 2002.
Struve et al., Neurotoxicology, 22(3):375-385, 2001.
Suarez and Darveau, J. Exp. Biol. 208:1627-1634, 2005.
Sundarrajan et al., Macromolecules, 35:3331, 2002.
Supuran et al., Med. Res. Rev., 23(2):146-189, 2003.
Sweeney, In: A Survey of Compounds from the Antiradiation Drug Development Program of the U.S. Army Medical Research and Development Command. Walter Reed Army Institute of Research, Washington D.C., 1979.
Teodoro and OFarrell, EMBO J., 22(3):580-587, 2003.
The Hypothermia After Cardiac Arrest Study Group et al., 2002.
Tisherman, Crit. Care Med., 32(2): 546-S50, 2004.
Van Voorhies et al., J. Exp. Biol., 203 (Pt 16):2467-2478, 2000.
Wang et al., 1992
Wang et al., 1993
Wang et al., 1994
Wang, FASEB J., 16(13):1792-1798, 2002.
Yaffe et al., Crit. Care Med., 32(2):S51-55, 2004.
Yaghi et al., Nature, 423(6941):705-714, 2003.
Yang et al., J. Agric. Food Chem., 52:7051, 2004.
Yoshikawa et al., J. Biochem. (Tokyo), 71:859-872, 1972.
Zhang et al., J. Appl. Physiol. 96(1):392-397, 2004.
Zhang et al., J. Org. Chem. 63:5314, 1998.
Ziegler, Ann. Rev. Biochem. 54, 305, 1985.

1.-109. (canceled)

110. A pharmaceutical composition comprising a chalcogenide of Formula (I) or (IV), or salt or prodrug thereof, and a pharmaceutically acceptable excipient, wherein Formula (I) and (IV) comprise: 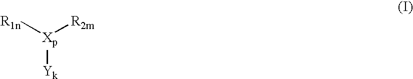
wherein X is N, O, Po, S, Se, or Te;
Y is N or O;
R₁ is H, C, lower alkyl, a lower alcohol, or CN;
R₂ is H, C, lower alkyl, or a lower alcohol, or CN;
n is 0 or 1;
m is 0 or 1;
k is 0, 1, 2, 3, or 4; and,
p is 1 or 2; 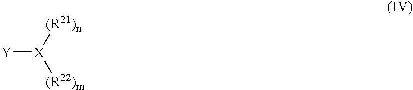
wherein:
X is N, O, P, Po, S, Se, Te, O—O, Po—Po, S—S, Se—Se, or Te—Te;
n and m are independently 0 or 1; and
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, heteroaryloxy, cycloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, or —R²³R²⁴, wherein R²³ is S, SS, Po, Po—Po, Se, Se—Se, Te, or Te—Te, and R²⁴ is defined as for R²¹ herein, or Y is 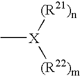
wherein X, R²¹ and R²², are as defined herein.

111. The pharmaceutical composition of claim 110, wherein the chalcogenide comprises sulfur.

112. The pharmaceutical composition of claim 110, wherein said salt is a sulfide salt selected from the group consisting of sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS).

113. The pharmaceutical composition of claim 110, wherein the chalcogenide is dissolved in or bubbled into a liquid.

114. The pharmaceutical composition of claim 110, wherein the chalcogenide is a gas, semi-solid liquid, liquid, or solid.

115. The pharmaceutical composition of claim 110, wherein the chalcogenide is selected from the group consisting of H₂S, H₂Se, H₂Te and H₂Po.

116. The pharmaceutical composition of claim 115, wherein the chalcogenide is gaseous H₂S.

117. A method for treating or preventing hemorrhagic shock in a subject comprising providing an effective amount of a chalcogenide of Formula I or Formula IV, or salt or prodrug thereof, wherein Formula I and Formula IV comprise: 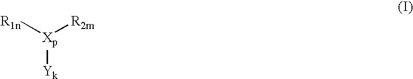
wherein X is N, O, Po, S, Se, or Te;
Y is N or O;
R₁ is H, C, lower alkyl, a lower alcohol, or CN;
R₂ is H, C, lower alkyl, or a lower alcohol, or CN;
n is 0 or 1;
m is 0 or 1;
k is 0, 1, 2, 3, or 4; and,
p is 1 or 2; 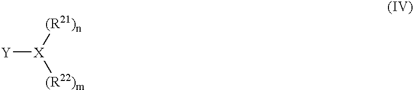
wherein:
X is N, O, P, Po, S, Se, Te, 0-0, Po—Po, S—S, Se—Se, or Te—Te;
n and m are independently 0 or 1; and
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, heteroaryloxy, cycloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, or —R²³R²⁴, wherein R²³ is S, SS, Po, Po—Po, Se, Se—Se, Te, or Te—Te, and R²⁴ is defined as for R²¹ herein, or Y is 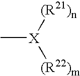
wherein X, R²¹ and R²², are as defined herein.

118. The method of claim 117, wherein the chalcogenide is further defined as gaseous H₂S.

119. The method of claim 117, wherein the subject is provided the chalcogenide through inhalation, injection, catheterization, immersion, lavage, perfusion, topical application, absorption, adsorption, or oral administration.

120. The method of claim 117, wherein the subject is provided the chalcogenide by administration to the subject intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

121. The method of claim 117, wherein the chalcogenide is provided to the subject using a nebulizer.

122. The method of claim 117, wherein the chalcogenide is provided to the subject in a pharmaceutical composition.

123. The method of claim 117, further comprising identifying a subject in need of treatment.

124. A method of preventing an organism from bleeding to death comprising providing to the bleeding organism an effective amount of a chalcogenide of Formula (I) or (IV), or salt or prodrug thereof, to prevent death, wherein Formula (I) and (IV) comprise: 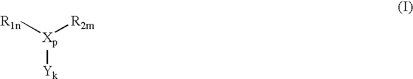
wherein X is N, O, Po, S, Se, or Te;
Y is N or O;
R₁ is H, C, lower alkyl, a lower alcohol, or CN;
R₂ is H, C, lower alkyl, or a lower alcohol, or CN;
n is 0 or 1;
m is 0 or 1;
k is 0, 1, 2, 3, or 4; and,
p is 1 or 2; 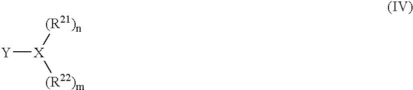
wherein:
X is N, O, P, Po, S, Se, Te, 0-0, Po—Po, S—S, Se—Se, or Te—Te;
n and m are independently 0 or 1; and
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, heteroaryloxy, cycloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, or —R²³R²⁴, wherein R²³ is S, SS, Po, Po—Po, Se, Se—Se, Te, or Te—Te, and R²⁴ is defined as for R²¹ herein, or Y is 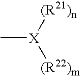
wherein X, R²¹ and R²², are as defined herein.

125. The method of claim 124, wherein the organism goes into hemorrhagic shock.

126. The method of claim 124, further comprising placing the organism under hypoxic conditions.

127. A method for preventing or treating shock in a subject comprising providing to a subject an effective amount of a chalcogenide of Formula (I) or (IV), or salt or prodrug thereof, wherein Formula (I) and (IV) comprise: 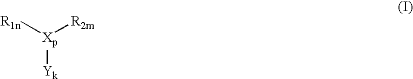
wherein X is N, O, Po, S, Se, or Te;
Y is N or O;
R₁ is H. C, lower alkyl, a lower alcohol, or CN;
R₂ is H, C, lower alkyl, or a lower alcohol, or CN;
n is 0 or 1;
m is 0 or 1;
k is 0, 1, 2, 3, or 4; and,
p is 1 or 2; 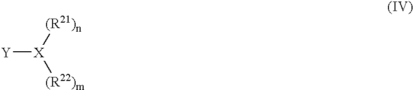
wherein:
X is N, O, P, Po, S, Se, Te, O—O, Po—Po, S—S, Se—Se, or Te—Te;
n and m are independently 0 or 1; and
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, heteroaryloxy, cycloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, or —R²³R²⁴, wherein R²³ is S, SS, Po, Po—Po, Se, Se—Se, Te, or Te—Te, and R²⁴ is defined as for R²¹ herein, or Y is 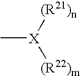
wherein X, R²¹ and R²², are as defined herein.

128. The method of claim 127, wherein the subject is bleeding or is at risk for bleeding.

129. The method of claim 127, wherein the subject is at risk for hemorrhagic shock.

130. A method of preventing or reducing cellular damage in a subject due to a disease or adverse medical condition, comprising providing to the subject an effective amount of a chalcogenide of Formula (I) or (IV), or salt or prodrug thereof, wherein Formula (I) and (IV) comprise: 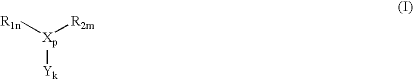
wherein X is N, O, Po, S, Se, or Te;
Y is N or O;
R¹ is H, C, lower alkyl, a lower alcohol, or CN;
R₂ is H, C, lower alkyl, or a lower alcohol, or CN;
n is 0 or 1;
m is 0 or 1;
k is 0, 1, 2, 3, or 4; and,
p is 1 or 2; 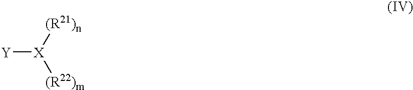
wherein:
X is N, O, P, Po, S, Se, Te, O—O, Po—Po, S—S, Se—Se, or Te—Te;
n and m are independently 0 or 1; and
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, heteroaryloxy, cycloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, or —R²³R²⁴, wherein R²³ is S, SS, Po, Po—Po, Se, Se—Se, Te, or Te—Te, and R²⁴ is defined as for R²¹ herein, or Y is 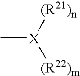
wherein X, R²¹ and R²², are as defined herein.

131. A method of protecting biological subject from an injury, the onset or progression of a disease, or death comprising providing to the subject, before the injury, the onset or progression of a disease, or death, an effective amount of an effective amount of a chalcogenide of Formula (I) or (IV), or salt or prodrug thereof, wherein Formula (I) and (IV) comprise: 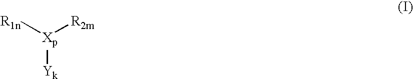
wherein X is N, O, Po, S, Se, or Te;
Y is N or O;
R₁ is H, C, lower alkyl, a lower alcohol, or CN;
R₂ is H, C, lower alkyl, or a lower alcohol, or CN;
n is 0 or 1;
m is 0 or 1;
k is 0, 1, 2, 3, or 4; and,
p is 1 or 2; 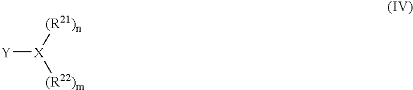
wherein:
X is N, O, P, Po, S, Se, Te, O—O, Po—Po, S—S, Se—Se, or Te—Te;
n and m are independently 0 or 1; and
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, heteroaryloxy, cycloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, or —R²³R²⁴, wherein R²³ is S, SS, Po, Po—Po, Se, Se—Se, Te, or Te—Te, and R²⁴ is defined as for R²¹ herein, or Y is 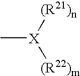
wherein X, R²¹ and R²², are as defined herein,
wherein the effective amount is less than an amount that can induce stasis in the biological subject.

132. A method for enhancing survivability of biological matter comprising:
(a) providing to the biological matter an effective amount of at least one chalcogenide of Formula (I) or (IV), or salt or prodrug thereof, wherein Formula (I) and (IV) comprise: 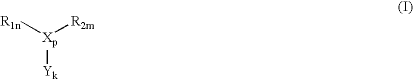
wherein X is N, O, Po, S, Se, or Te;
Y is N or O;
R¹ is H, C, lower alkyl, a lower alcohol, or CN;
R₂ is H, C, lower alkyl, or a lower alcohol, or CN;
n is 0 or 1;
m is 0 or 1;
k is 0, 1, 2, 3, or 4; and,
p is 1 or 2; 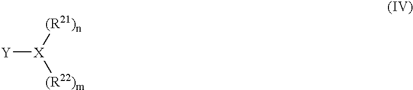
wherein:
X is N, O, P, Po, S, Se, Te, O—O, Po—Po, S—S, Se—Se, or Te—Te;
n and m are independently 0 or 1; and
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, heteroaryloxy, cycloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, or —R²³R²⁴, wherein R²³ is S, SS, Po, Po—Po, Se, Se—Se, Te, or Te—Te, and R²⁴ is defined as for R²¹ herein, or Y is 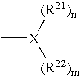
wherein X, R²¹ and R²², are as defined herein; and
(b) placing the biological matter under hypoxic conditions.

133. A method for reversibly inhibiting metabolism in an organism comprising:
(a) providing to the biological matter an effective amount of a chalcogenide of Formula (I) or (IV), or salt or prodrug thereof, wherein Formula (I) and (IV) comprise: 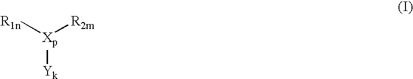
wherein X is N, O, Po, S, Se, or Te;
Y is N or O;
R₁ is H, C, lower alkyl, a lower alcohol, or CN;
R₂ is H, C, lower alkyl, or a lower alcohol, or CN;
n is 0 or 1;
m is 0 or 1;
k is 0, 1, 2, 3, or 4; and,
p is 1 or 2; 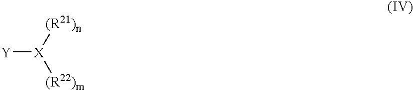
wherein:
X is N, O, P, Po, S, Se, Te, O—O, Po—Po, S—S, Se—Se, or Te—Te;
n and m are independently 0 or 1; and
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, heteroaryloxy, cycloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, or —R²³R²⁴, wherein R²³ is S, SS, Po, Po—Po, Se, Se—Se, Te, or Te—Te, and R²⁴ is defined as for R²¹ herein, or Y is 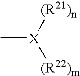
wherein X, R²¹ and R²², are as defined herein; and
(b) placing the biological matter under hypoxic conditions.
